(19) United States
(12) Patent Application Publication
(10) Pub. No.: US 2015/0030757 A1
(43) Pub. Date: Jan. 29, 2015

(54) PERIPHERAL STENTS HAVING LAYERS

(71) Applicant: MICELL TECHNOLOGIES, INC., Durham, NC (US)

(72) Inventors: James B. McClain, Raleigh, NC (US); Douglas Taylor, Franklinton, NC (US)

(21) Appl. No.: 14/457,609

(22) Filed: Aug. 12, 2014

Related U.S. Application Data
(63) Continuation of application No. 12/729,156, filed on Mar. 22, 2010, now abandoned.
(60) Provisional application No. 61/162,569, filed on Mar. 23, 2009.
(60) Provisional application No. 61/243,955, filed on Sep. 18, 2009.
(60) Provisional application No. 61/226,239, filed on Jul. 16, 2009.

(51) Int. Cl. A61L 31/10; A61L 31/16; A61F 2/86

(52) U.S. Cl. CPC A61L 31/10; A61F 2/86; A61L 31/16; A61L 2420/02; A61L 2420/08; A61L 2420/06; A61L 2300/216; A61F 2240/001; A61F 2250/001
USPC 427/2.21

(57) ABSTRACT

Provided herein is a coated coronary stent, comprising: a. stent; b. a plurality of layers deposited on said stent to form said coronary stent; wherein at least one of said layers comprises a bioabsorbable polymer and at least one of said layers comprises one or more active agents; wherein at least part of the active agent is in crystalline form.

### CROSS-REFERENCE

This application is a continuation of U.S. application Ser. No. 12/729,156, filed Mar. 22, 2010, which claims the benefit of U.S. Provisional Application No. 61/162,569, filed Mar. 23, 2009; U.S. Provisional Application No. 61/243,955, filed Sep. 18, 2009; and U.S. Provisional Application No. 61/226,239, filed Jul. 16, 2009, each of which are incorporated herein by reference in their entirety.

### BACKGROUND OF THE INVENTION

The present invention relates to methods for forming stents comprising a bioabsorbable polymer and a pharmaceutical or biological agent in powder form onto a substrate.

It is desirable to have a drug-eluting stent with minimal physical, chemical and therapeutic legacy in the vessel after a proscribed period of time. This period of time is based on the effective healing of the vessel after opening the blockage by PCI/stenting (currently believed by leading clinicians to be 6-18 months).

It is also desirable to have drug-eluting stents of minimal cross-sectional thickness for (a) flexibility of deployment (b) access to small and large vessels (c) minimized intrusion into the vessel wall and blood.

### SUMMARY OF THE INVENTION

Provided herein is a coated stent having a plurality of stent struts for delivery to a body lumen comprising a stent and a coating comprising a pharmaceutical agent and a polymer wherein at least part of the drug is in crystalline form and wherein the coating is substantially resistant to stent strut breakage. The body lumen may include a peripheral body lumen or a coronary body lumen.

In some embodiments, the polymer comprises a durable polymer. The polymer may include a cross-linked durable polymer. The polymer may include a thermoset material. The polymer may provide radial strength for the coated stent. The polymer may provide durability for the coated stent. The polymer may be impenetrable by a broken strut of the stent.

In some embodiments, the polymer comprises a bioabsorbable polymer. In some embodiments, the polymer comprises a cross-linked bioabsorbable polymer.

In some embodiments, the coating comprises a plurality of layers deposited on said stent to form said coated stent. The coating may comprise five layers deposited as follows: a first polymer layer, a first drug layer, a second polymer layer, a second drug layer and a third polymer layer. In some embodiments, the drug and polymer are in the same layer; in separate layers or form overlapping layers. In some embodiments, plurality of layers comprises at least 4 or more layers. In some embodiments, the plurality of layers comprises 10, 20, 50, or 100 layers. In some embodiments, the plurality of layers comprises at least one of: at least 10, at least 20, at least 50, and at least 100 layers. In some embodiments, the plurality of layers comprises alternate drug and polymer layers. The drug layers may be substantially free of polymer and/or the polymer layers may be substantially free of drug.

In some embodiments the coating comprises a fiber reinforcement. The fiber reinforcement may comprise a natural or a synthetic fiber. Examples of the fiber reinforcement may include any biocompatible fiber known in the art. This may, for non-limiting example, include any reinforcing fiber from silk to catgut to polymers to olefins to acrylates. The fiber may be deposited according to methods disclosed herein, including by RESS. The concentration for a reinforcing fiber that is or comprises a polymer may be any concentration of a fiber forming polymer from 5 to 50 miligrams per milliliter and deposited according to the RESS process. The fiber may comprise a length to diameter ratio of at least 3:1, in some embodiments. The fiber may comprise lengths of at least 200 nanometers. The fiber may comprise lengths of up to 5 micrometers in certain embodiments. The fiber may comprise lengths of 200 nanometers to 5 micrometers, in some embodiments.

Provided herein is a coated stent having a plurality of stent struts for delivery to a body lumen comprising a stent and a coating comprising a pharmaceutical agent and a polymer wherein at least part of the drug is in crystalline form and wherein the coating provides a release profile whereby the pharmaceutical agent is released over a period longer than two weeks. The body lumen may include a peripheral body lumen, and/or a coronary body lumen.

In some embodiments, the coating provides a release profile whereby the drug is released over a period longer than 1 month. In some embodiments, the coating provides a release profile whereby the drug is released over a period longer than 2 months. In some embodiments, the coating provides a release profile whereby the drug is released over a period longer than 3 months. In some embodiments, the coating provides a release profile whereby the drug is released over a period longer than 4 months. In some embodiments, the coating provides a release profile whereby the drug is released over a period longer than 6 months. In some embodiments, the coating provides a release profile whereby the pharmaceutical agent is released over a period longer than twelve months.

In some embodiments, over 1% of said pharmaceutical agent coated on said stent is delivered to the vessel. In some embodiments, over 2% of said pharmaceutical agent coated on said stent is delivered to the vessel. In some embodiments, over 5% of said pharmaceutical agent coated on said stent is delivered to the vessel. In some embodiments, over 10% of said pharmaceutical agent coated on said stent is delivered to the vessel. In some embodiments, over 25% of said pharmaceutical agent coated on said stent is delivered to the vessel. In some embodiments, over 50% of said pharmaceutical agent coated on said stent is delivered to the vessel.

In some embodiments, the agent and polymer coating has substantially uniform thickness and drug in the coating is substantially uniformly dispersed within the agent and polymer coating.

In some embodiments, the coated stent provides an elution profile wherein about 10% to about 50% of drug is eluted at week 20 after the stent is implanted in a subject under physiological conditions, about 25% to about 75% of drug is eluted at week 30 and about 50% to about 100% of drug is eluted at week 50.

Some embodiments of the coating further comprise an anti-inflammatory agent.

In some embodiments, the macrolide-polymer coating comprises one or more resorbable polymers. In some embodiments, one or more resorbable polymers are selected from PLGA (poly(lactide-co-glycolide); DLPLA—poly(dl-lactide); LPLA—poly(1-lactide); PGA polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

In some embodiments, the polymer is 50/50 PLGA.

Provided herein is a coated stent having a plurality of stent struts for delivery to a body lumen comprising a stent and a coating comprising a pharmaceutical agent and a polymer wherein at least part of the drug is in crystalline form and wherein said coating is substantially conformal to the stent struts when the coated stent is in an expanded state. The body lumen may include a peripheral body lumen, and/or a coronary body lumen.

In some embodiments, the coating is applied when the stent is in a collapsed state. In some embodiments, the coated stent has a radial expansion ratio of about 1 in a collapsed state up to about 3.0 in the expanded state. In some embodiments, the coated stent has a radial expansion ratio of about 1 in a collapsed state up to about 4.0 in the expanded state. In some embodiments, the coated stent has a radial expansion ratio of about 1 in a collapsed state up to about 5.0 in the expanded state. In some embodiments, the coated stent has a radial expansion ratio of about 1 in a collapsed state up to about 6.0 in the expanded state. In some embodiments, the coated stent has a radial expansion ratio of about 1 in a collapsed state to over about 3.0 in the expanded state. In some embodiments, the coated stent has a radial expansion ratio of about 1 in a collapsed state to over about 4.0 in the expanded state.

In some embodiments, the pharmaceutical agent comprises one or more of an antirestenotic agent, antidiabetic, analgesic, antiinflammatory agent, antirheumatic, antihypotensive agent, antihypertensive agent, psychoactive drug, tranquillizer, antiemetic, muscle relaxant, glucocorticoid, agent for treating ulcerative colitis or Crohn's disease, antiallergic, antibiotic, antiepileptic, anticoagulant, antimycotic, antitussive, arteriosclerosis remedy, diuretic, protein, peptide, enzyme, enzyme inhibitor, gout remedy, hormone and inhibitor thereof, cardiac glycoside, immunotherapeutic agent and cytokine, laxative, lipid-lowering agent, migraine remedie, mineral product, otological, anti parkinson agent, thyroid therapeutic agent, spasmolytic, platelet aggregation inhibitor, vitamin, cytostatic and metastasis inhibitor, phytopharmaceutical, chemotherapeutic agent and amino acid, acarbose, antigen, beta-receptor blocker, non-steroidal antiinflammatory drug [NSAIDs], cardiac glycosides acetylsalicylic acid, virustatic, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, and zotipine.

In some embodiments, the pharmaceutical agent comprises a macrolide immunosuppressive (limus) drug. The macrolide immunosuppressive drug may comprise one or more of rapamycin, biolimus (biolimus A9), 4O—O-(2-Hydroxyethyl)rapamycin (everolimus), 4O—O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin 4O—O-(2-Hydroxy)ethoxycar-bonylmethyl-rapamycin, 4O—O-(3-Hydroxy)propyl-rapamycin 4O—O-(6-Hydroxy)hexyl-rapamycin 4O—O-[2-(2-Hydroxy)ethoxy]ethyl-rapamycin 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the coating further comprises an anti-inflammatory agent.

In some embodiments, at least part of said drug forms a phase separate from one or more phases formed by said polymer.

In some embodiments, the drug is at least 50% crystalline. In some embodiments, the drug is at least 75% crystalline. In some embodiments, the drug is at least 90% crystalline. In some embodiments, the drug is at least 95% crystalline. In some embodiments, the drug is at least 99% crystalline.

In some embodiments, the polymer is a mixture of two or more polymers. In some embodiments, the mixture of polymers forms a continuous film around particles of drug. The two or more polymers may be intimately mixed. The mixture may comprise no single polymer domain larger than about 20 nm. Each polymer in said mixture may comprise a discrete phase. Discrete phases formed by said polymers in said mixture may be larger than about 10 nm. Discrete phases formed by said polymers in said mixture may be larger than about 50 nm.

In some embodiments, the stent comprises at least one of stainless steel, a cobalt-chromium alloy, tantalum, platinum, Nitinol™, gold, a NiTi alloy, and a thermoplastic polymer.

In some embodiments, the stent is formed from a metal alloy.

In some embodiments, the stent is capable of retaining its expanded condition upon the expansion thereof.

In some embodiments, the stent is formed from a material that plastically deforms when subjected to at least 4 atmospheres of pressure. In some embodiments, the stent is formed from a material that plastically deforms when subjected to at least 2 atmospheres of pressure. In some embodiments, the stent is formed from a material that plastically deforms when subjected to at least 5 atmospheres of pressure. In some embodiments, the stent is formed from a material that plastically deforms when subjected to at least 6 atmospheres of pressure.

In some embodiments, the stent is formed from a material that is capable of self-expansion in the body lumen.

In some embodiments, the stent is formed from a super-elastic metal alloy which transforms from an austenitic state to a martensitic state in the body lumen. In some embodiments, the stent is formed from a super-elastic metal alloy that is capable of deformation from a martensitic state to an austenitic state when the stent is mounted on a catheter. In some embodiments, the stent exhibits linear pseudoelasticity when stressed. In some embodiments, the stent is formed from a super-elastic metal alloy having a transformation temperature greater than a mammalian body temperature.

In some embodiments, at least one of the stent and the polymer is formed of a radiopaque material. In some embodiments, the stent comprises at least one of: iridium, platinum, gold, rhenium, tungsten, palladium, rhodium, tantalum, silver, ruthenium, chromium, iron, cobalt, vanadium, manganese, boron, copper, aluminum, niobium, zirconium, and hafnium.

In some embodiments, heparin is attached to the stent by reaction with an aminated silane. In some embodiments, the stent is coated with a silane monolayer.

In some embodiments, onset of heparin anti-coagulant activity is obtained at week 3 or later. In some embodiments, heparin anti-coagulant activity remains at an effective level at least 90 days after onset of heparin activity. In some embodiments, heparin anti-coagulant activity remains at an effective level at least 120 days after onset of heparin activity. In some embodiments, the heparin anti-coagulant activity remains at an effective level at least 200 days after onset of heparin activity.

In some embodiments, the stent is adapted for delivery to at least one of a peripheral artery, a peripheral vein, a carotid artery, a vein, an aorta, and a biliary duct. In some embodiments, the stent is adapted for delivery to a superficial femoral artery. The stent may be adapted for delivery to a tibial artery. The stent may be adapted for delivery to a renal artery. The stent may be adapted for delivery to an iliac artery. The stent may be adapted for delivery to a bifurcated vessel. The stent is adapted for delivery to a vessel having a side branch at an intended delivery site of the vessel. The stent is adapted for delivery to the side branch of the vessel.

Provided herein is a method for preparing a coated stent for delivery to a body lumen comprising the following steps: providing a stent, forming a coating comprising a pharmaceutical agent and a polymer on the stent wherein at least part of the drug is in crystalline form, and wherein the coating is substantially resistant to stent strut breakage. The body lumen may include a peripheral body lumen, and/or a coronary body lumen.

Provided herein is a method for preparing a coated stent for delivery to a body lumen comprising the following steps: providing a stent; forming a coating comprising a pharmaceutical agent and a polymer on the stent wherein at least part of the drug is in crystalline form, and wherein the coating provides a release profile whereby the pharmaceutical agent is released over a period longer than 2 weeks. The body lumen may include a peripheral body lumen, and/or a coronary body lumen.

Provided herein is a method for preparing a coated stent for delivery to a body lumen comprising the following steps: providing a stent; forming a coating comprising a pharmaceutical agent and a polymer on the stent wherein at least part of the drug is in crystalline form, and wherein said coating is substantially conformal to the stent struts when the coated stent is in an expanded state. The body lumen may include a peripheral body lumen, and/or a coronary body lumen.

In some embodiments, forming the coating comprises depositing the drug in dry powder form.

In some embodiments, forming the coating comprises depositing the polymer in dry powder form.

In some embodiments, forming the coating comprises depositing the polymer by an e-SEDS process.

In some embodiments, forming the coating comprises depositing the polymer by an e-RESS process.

In some embodiments, the method comprises sintering said coating under conditions that do not substantially modify the morphology of said drug.

In some embodiments, the pharmaceutical agent comprises one or more of an antirestenotic agent, antidiabetic, analgesic, antiinflammatory agent, antirheumatic, antihypotensive agent, antihypertensive agent, psychoactive drug, tranquillizer, antiemetic, muscle relaxant, glucocorticoid, agent for treating ulcerative colitis or Crohn's disease, antiallergic, antibiotic, antiepileptic, anticoagulant, antimycotic, antitussive, arteriosclerosis remedy, diuretic, protein, peptide, enzyme, enzyme inhibitor, gout remedy, hormone and inhibitor thereof, cardiac glycoside, immunotherapeutic agent and cytokine, laxative, lipid-lowering agent, migraine remedie, mineral product, otological, anti parkinson agent, thyroid therapeutic agent, spasmolytic, platelet aggregation inhibitor, vitamin, cytostatic and metastasis inhibitor, phytopharmaceutical, chemotherapeutic agent and amino acid, acarbose, antigen, beta-receptor blocker, non-steroidal antiinflammatory drug {NSAIDs], cardiac glycosides acetylsalicylic acid, virustatic, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, and zotipine.

In some embodiments, the pharmaceutical agent comprises a macrolide immunosuppressive drug, and the macrolide immunosuppressive drug comprises one or more of rapamycin, biolimus (biolimus A9), 4O—O-(2-Hydroxyethyl)rapamycin (everolimus), 4O—O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin 4O—O-(2-Hydroxy)ethoxycar-bonylmethyl-rapamycin, 4O—O-(3-Hydroxy)propyl-rapamycin 4O—O-(6-Hydroxy)hexyl-rapamycin 4O—O-[2-(2-Hydroxy)ethoxy]ethyl-rapamycin 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the polymer comprises a bioabsorbable polymer and wherein forming the coating comprises depositing the bioabsorbable polymer in dry powder form.

In some embodiments, one or more bioabsorbable polymers are selected from PLGA (poly(lactide-co-glycolide); DLPLA—poly(dl-lactide); LPLA—poly(1-lactide); PGA polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone).

In some embodiments, the bioabsorbable polymer is cross-linked. In some embodiments, the polymer comprises a durable polymer, and wherein forming the coating comprises depositing the durable polymer in dry powder form. In some embodiments, the durable polymer is cross-linked. In some embodiments, the durable polymer comprises a thermoset material.

In some embodiments, the forming the coating comprises depositing a first polymer layer, depositing a first drug layer, depositing a second polymer layer, depositing a second drug layer and depositing a third polymer layer. In some embodiments, the forming the coating comprises depositing a plurality of layers on said stent to form said coated stent. In some embodiments, the drug and polymer are in the same layer; in separate layers or form overlapping layers. In some embodiments, forming the coating comprises depositing at least 4 or more layers. In some embodiments, forming the coating comprises depositing 10, 20, 50, or 100 layers. In some embodiments, forming the coating comprises depositing at least one of: at least 10, at least 20, at least 50, and at least 100 layers. In some embodiments, forming the coating comprises depositing alternate drug and polymer layers. In some embodiments, forming the coating comprises depositing drug layers that are substantially free of polymer and the polymer layers are substantially free of drug.

In some embodiments the coating comprises a fiber reinforcement. The fiber reinforcement may comprise a natural or a synthetic fiber. Examples of the fiber reinforcement may include any biocompatible fiber known in the art. This may, for non-limiting example, include any reinforcing fiber from silk to catgut to polymers to olefins to acrylates. The fiber may be deposited according to methods disclosed herein, including by RESS. The concentration for a reinforcing fiber that is or comprises a polymer may be any concentration of a fiber forming polymer from 5 to 50 miligrams per milliliter and deposited according to the RESS process. The fiber may comprise a length to diameter ratio of at least 3:1, in some embodiments. The fiber may comprise lengths of at least 200 nanometers. The fiber may comprise lengths of up to 5 micrometers in certain embodiments. The fiber may comprise lengths of 200 nanometers to 5 micrometers, in some embodiments.

In some embodiments, the stent comprises at least one of stainless steel, a cobalt-chromium alloy, tantalum, platinum, Nitinol™, gold, a NiTi alloy, and a thermoplastic polymer. In some embodiments, stent is formed from a metal alloy. In some embodiments, the stent is capable of retaining its expanded condition upon the expansion thereof. In some embodiments, the stent is formed from a material that plastically deforms when subjected to at least 4 atmospheres of pressure. In some embodiments, the stent is formed from a material that is capable of self-expansion in the body lumen. In some embodiments, the stent is formed from a super-elastic metal alloy which transforms from an austenitic state to a martensitic state in the body lumen. In some embodiments, the stent is formed from a super-elastic metal alloy that is capable of deformation from a martensitic state to an austenitic state when the stent is mounted on a catheter. In some embodiments, the stent exhibits linear pseudoelasticity when stressed. In some embodiments, the stent is formed from a super-elastic metal alloy having a transformation temperature greater than a mammalian body temperature.

In some embodiments, at least one of the stent and the polymer is formed of a radiopaque material. In some embodiments, the stent comprises at least one of: iridium, platinum, gold, rhenium, tungsten, palladium, rhodium, tantalum, silver, ruthenium, chromium, iron, cobalt, vanadium, manganese, boron, copper, aluminum, niobium, zirconium, and hafnium.

In some embodiments, comprising forming a silane layer on the stent, and covalently attaching heparin to the silane layer. In some embodiments, onset of heparin anti-coagulant activity is obtained at week 3 or later. In some embodiments, heparin anti-coagulant activity remains at an effective level at least 90 days after onset of heparin activity. In some embodiments, heparin anti-coagulant activity remains at an effective level at least 120 days after onset of heparin activity. In some embodiments, heparin anti-coagulant activity remains at an effective level at least 200 days after onset of heparin activity.

In some embodiments, the polymer is 50/50 PLGA.

In some embodiments, at least part of said drug forms a phase separate from one or more phases formed by said polymer.

In some embodiments, the drug is at least 50% crystalline. In some embodiments, the drug is at least 75% crystalline. In some embodiments, the drug is at least 90% crystalline. In some embodiments, the drug is at least 95% crystalline. In some embodiments, the drug is at least 99% crystalline.

In some embodiments, the polymer is a mixture of two or more polymers. In some embodiments, the mixture of polymers forms a continuous film around particles of drug. In some embodiments, the two or more polymers are intimately mixed. In some embodiments, the mixture comprises no single polymer domain larger than about 20 nm. In some embodiments, each polymer in said mixture comprises a discrete phase. In some embodiments, the discrete phases formed by said polymers in said mixture are larger than about 10 nm. In some embodiments, the discrete phases formed by said polymers in said mixture are larger than about 50 nm.

In some embodiments, forming coating is done when the stent is in a collapsed state. In some embodiments, the coated stent has a radial expansion ratio of about 1 in a collapsed state up to about 3.0 in the expanded state. In some embodiments, the coated stent has a radial expansion ratio of about 1 in a collapsed state up to about 4.0 in the expanded state. In some embodiments, the coated stent has a radial expansion ratio of about 1 in a collapsed state up to about 5.0 in the expanded state. In some embodiments, the coated stent has a radial expansion ratio of about 1 in a collapsed state up to about 6.0 in the expanded state. In some embodiments, the coated stent has a radial expansion ratio of about 1 in a collapsed state to over about 3.0 in the expanded state. In some embodiments, the coated stent has a radial expansion ratio of about 1 in a collapsed state to over about 4.0 in the expanded state.

In some embodiments, the stent is adapted for delivery to at least one of a peripheral artery, a peripheral vein, a carotid artery, a vein, an aorta, and a biliary duct. In some embodiments, the stent is adapted for delivery to a superficial femoral artery. The stent may be adapted for delivery to a tibial artery. The stent may be adapted for delivery to a renal artery. The stent may be adapted for delivery to an iliac artery. The stent may be adapted for delivery to a bifurcated vessel. The stent is adapted for delivery to a vessel having a side branch at an intended delivery site of the vessel. The stent is adapted for delivery to the side branch of the vessel.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a Rapamycin Elution Profile of coated stents (PLGA/Rapamycin coatings) where the elution profile was determined by a static elution media of 5% EtOH/water, pH 7.4, 37° C. via UV-Vis test method as described in Example 11b of coated stents described therein.

FIG. 2 depicts a Rapamycin Elution Profile of coated stents (PLGA/Rapamycin coatings) where the elution profile was determined by static elution media of 5% EtOH/water, pH 7.4, 37° C. via a UV-Vis test method as described in Example 11b of coated stents described therein; FIG. 2 depicts AS1 and AS2 as having statistically different elution profiles; AS2 and AS2b have stastically different profiles; AS1 and AS1b are not statistically different; and AS2 and AS1(213) begin to converge at 35 days; FIG. 2 suggests that the coating thickness does not affect elution rates form 3095 polymer, but does affect elution rates from the 213 polymer.

FIG. 3 depicts Rapamycin Elution Rates of coated stents (PLGA/Rapamycin coatings) where the static elution profile was compared with agitated elution profile by an elution media of 5% EtOH/water, pH 7.4, 37° C. via a UV-Vis test method a UV-Vis test method as described in Example 11b of coated stents described therein; FIG. 3 depicts that agitation in elution media increases the rate of elution for AS2 stents, but is not statistically significantly different for AS1 stents; the profiles are based on two stent samples.

FIG. 4 depicts Rapamycin Elution Profile of coated stents (PLGA/Rapamycin coatings) where the elution profile by 5% EtOH/water, pH 7.4, 37° C. elution buffer was compare with the elution profile using phosphate buffer saline pH 7.4, 37° C.; both profiles were determined by a UV-Vis test method as described in Example 11b of coated stents described therein; FIG. 4 depicts that agitating the stent in elution media increases the elution rate in phosphate buffered saline, but the error is much greater.

FIG. 5 depicts Rapamycin Elution Profile of coated stents (PLGA/Rapamycin coatings) where the elution profile was determined by a 20% EtOH/phosphate buffered saline, pH 7.4, 37° C. elution buffer and a HPLC test method as described in Example 11c described therein, wherein the elution time (x-axis) is expressed linearly.

FIG. 6 depicts Rapamycin Elution Profile of coated stents (PLGA/Rapamycin coatings) where the elution profile was determined by a 20% EtOH/phosphate buffered saline, pH 7.4, 37° C. elution buffer and a HPLC test method as described in Example 11c of described therein, the elution time (x-axis) is expressed in logarithmic scale (i.e., log(time)).

FIG. 7 depicts Bioabsorbability testing of 50:50 PLGA-ester end group (MW˜19 kD) polymer coating formulations on stents by determination of pH Changes with Polymer Film Degradation in 20% Ethanol/Phosphate Buffered Saline as set forth in Example 3 described herein.

FIG. 8 depicts Bioabsorbability testing of 50:50 PLGA-carboxylate end group (MW 10 kD) PLGA polymer coating formulations on stents by determination of pH Changes with Polymer Film Degradation in 20% Ethanol/Phosphate Buffered Saline as set forth in Example 3 described herein.

FIG. 9 depicts Bioabsorbability testing of 85:15 (85% lactic acid, 15% glycolic acid) PLGA polymer coating formulations on stents by determination of pH Changes with Polymer Film Degradation in 20% Ethanol/Phosphate Buffered Saline as set forth in Example 3 described herein.

FIG. 10 depicts Bioabsorbability testing of various PLGA polymer coating film formulations by determination of pH Changes with Polymer Film Degradation in 20% Ethanol/Phosphate Buffered Saline as set forth in Example 3 described herein.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

Provided herein is a system that utilizes supercritical fluids (e-RESS) that is solvent free, processed at a low temperature and can incorporate multiple drugs. Since the drugs and/or polymers of the coating is processed “dry” (i.e. without solvents) there is no bleeding of layers in some embodiments. The processes of some embodiments results in excellent adhesion of layers and mechanical properties. The processes of some embodiments enables precision of layers and rapid batch processing.

Provided herein is a system capable of making novel devices. It enables laminate structures, and can form intricate and novel devices. Some embodiments of the laminate structures provide structural control without introducing new materials or a new delivery system. Such an embodiment has been demonstrated for a drug-eluted coating and/or coated membranes in the examples and figures provided herein.

Provided herein is a process comprising electrostatc coating wherein nano and microparticles of polymer(s) and/or drug(s) are electrostatically captured, dry upon a stent form (for nonlimiting example), The process may then comprise sintering wherein polymer nanoparticles are fused via SCF which includes no solvents and no high temperatures. The final material provides a smooth, adherently laminated layer with precise control over location of the drug(s) within the coating.

Provided herein is a coating and/or a proess that is mechanically effective with and/or without a base-coating on the substrate, for non-limiting example a parylene base-coat.

Provided herein is a coating on a substrate that is smooth, conformal, and mechanically adherent on a variety of substrates (e.g. various types of stents or other substrates). There are a wide range of drugs (e.g. rapamycin, paclitaxel, heparin, small molecules, or another active agent described herein) that can be used in accordance with the processes and within the coatings described herein, at least. In some embodiments, multiple and/or dissimilar drugs (e.g. paclitaxel and heparin) are used in the same coating and achieve effective and useful coatings. In some embodiments, stents coating and sintered according to processes noted herein result in a conformal and even film over all aspects of the substrate of the device.

Provided herein is a coating process and system that provides control over drug (pharmaceutical agent, active biological agent) morphology, for example in a pharmaceutical agent it may provide control over the crystallinity of the drug (i.e. control over whether the drug is crystalline or amorphous. Some embodiments maintain drug stability. Some embodiments have no effect on elution profiles as compared to commercial analogs of the same drugs. In one example, a rapamycin coating was produced using a process described herein and the peak area ratio between control samples and coated samples indicated no difference in the rate of rapamycin degradation, thus the drug (rapamycin) was maintained in its crystalline morphology.

Provided herein is a coating that is thin, conformal to the substrate, and defect free at a target thickness. For example, in one test, a coating was created according to the processes noted herein that produced a mean coating thickness of 10.2+/−0.2 microns, with no visible defects and which appeared conformal to the substrate.

Provided herein is a system and/or process that can control the drug placement within the coating on the substrate. For example in one test, drug was loaded purposefully in the center of a 10 micron DES (drug-eluting stent) coating. Confocal Raman Spectra indicated the drug peak in the center of the coating itself. In another test, drug was loaded equally throughout a 10 micron DES coating and SIMS testing of the coating surface show the coating evident in the surface (at least).

### DEFINITIONS

As used in the present specification, the following words and phrases are generally intended to have the meanings as set forth below, except to the extent that the context in which they are used indicates otherwise.

“Substrate” as used herein, refers to any surface upon which it is desirable to deposit a coating comprising a polymer and a pharmaceutical or biological agent, wherein the coating process does not substantially modify the morphology of the pharmaceutical agent or the activity of the biological agent. Biomedical implants are of particular interest for the present invention; however the present invention is not intended to be restricted to this class of substrates. Those of skill in the art will appreciate alternate substrates that could benefit from the coating process described herein, such as pharmaceutical tablet cores, as part of an assay apparatus or as components in a diagnostic kit (e.g. a test strip).

“Biomedical implant” as used herein refers to any implant for insertion into the body of a human or animal subject, including but not limited to stents (e.g., vascular stents), electrodes, catheters, leads, implantable pacemaker, cardioverter or defibrillator housings, joints, screws, rods, ophthalmic implants, femoral pins, bone plates, grafts, anastomotic devices, perivascular wraps, sutures, staples, shunts for hydrocephalus, dialysis grafts, colostomy bag attachment devices, ear drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc.

The implants may be formed from any suitable material, including but not limited to organic polymers (including stable or inert polymers and biodegradable polymers), metals, inorganic materials such as silicon, and composites thereof, including layered structures with a core of one material and one or more coatings of a different material. Substrates made of a conducting material facilitate electrostatic capture. However, the invention contemplates the use of electrostatic capture in conjunction with substrate having low conductivity or which non-conductive. To enhance electrostatic capture when a non-conductive substrate is employed, the substrate is processed while maintaining a strong electrical field in the vicinity of the substrate.

Subjects into which biomedical implants of the invention may be applied or inserted include both human subjects (including male and female subjects and infant, juvenile, adolescent, adult and geriatric subjects) as well as animal subjects (including but not limited to dog, cat, horse, monkey, etc.) for veterinary purposes and/or medical research.

In a preferred embodiment the biomedical implant is an expandable intraluminal vascular graft or stent (e.g., comprising a wire mesh tube) that can be expanded within a blood vessel by an angioplasty balloon associated with a catheter to dilate and expand the lumen of a blood vessel, such as described in U.S. Pat. No. 4,733,665 to Palmaz Shaz.

“Pharmaceutical agent” or “pharmaceutical agent” as used herein refers to any of a variety of drugs or pharmaceutical compounds that can be used as active agents to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the pharmaceutical agents of the invention may also comprise two or more drugs or pharmaceutical compounds. Pharmaceutical agents, include but are not limited to antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals, chemotherapeutic agents and amino acids. Examples of suitable active ingredients are acarbose, antigens, beta-receptor blockers, non-steroidal antiinflammatory drugs [NSAIDs], cardiac glycosides, acetylsalicylic acid, virustatics, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotipine, amphotericin B, caspofungin, voriconazole, resveratrol, PARP-1 inhibitors (including imidazoquinolinone, imidazpyridine, and isoquinolindione, tissue plasminogen activator (tPA), melagatran, lanoteplase, reteplase, staphylokinase, streptokinase, tenecteplase, urokinase, abciximab (ReoPro), eptifibatide, tirofiban, prasugrel, clopidogrel, dipyridamole, cilostazol, VEGF, heparan sulfate, chondroitin sulfate, elongated “RGD” peptide binding domain, CD34 antibodies, cerivastatin, etorvastatin, losartan, valartan, erythropoietin, rosiglitazone, pioglitazone, mutant protein Apo A1 Milano, adiponectin, (NOS) gene therapy, glucagon-like peptide 1, atorvastatin, and atrial natriuretic peptide (ANP), lidocaine, tetracaine, dibucaine, hyssop, ginger, turmeric, Arnica montana, helenalin, cannabichromene, rofecoxib, hyaluronidase, and the like. See, e.g., U.S. Pat. No. 6,897,205; see also U.S. Pat. No. 6,838,528; U.S. Pat. No. 6,497,729.

Examples of therapeutic agents employed in conjunction with the invention include, rapamycin, biolimus (biolimus A9), 4O—O-(2-Hydroxyethyl)rapamycin (everolimus), 4O—O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin 4O—O-(2-Hydroxy)ethoxycar-bonylmethyl-rapamycin, 4O—O-(3-Hydroxy)propyl-rapamycin 4O—O-(6-Hydroxy)hexyl-rapamycin 4O—O-[2-(2-Hydroxy)ethoxy]ethyl-rapamycin 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

The active ingredients may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers.

A “pharmaceutically acceptable salt” may be prepared for any pharmaceutical agent having a functionality capable of forming a salt, for example an acid or base functionality. Pharmaceutically acceptable salts may be derived from organic or inorganic acids and bases. The term “pharmaceutically-acceptable salts” in these instances refers to the relatively non-toxic, inorganic and organic base addition salts of the pharmaceutical agents.

“Prodrugs” are derivative compounds derivatized by the addition of a group that endows greater solubility to the compound desired to be delivered. Once in the body, the prodrug is typically acted upon by an enzyme, e.g., an esterase, amidase, or phosphatase, to generate the active compound.

“Stability” as used herein in refers to the stability of the drug in a polymer coating deposited on a substrate in its final product form (e.g., stability of the drug in a coated stent). The term stability will define 5% or less degradation of the drug in the final product form.

“Active biological agent” as used herein refers to a substance, originally produced by living organisms, that can be used to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the active biological agents of the invention may also comprise two or more active biological agents or an active biological agent combined with a pharmaceutical agent, a stabilizing agent or chemical or biological entity. Although the active biological agent may have been originally produced by living organisms, those of the present invention may also have been synthetically prepared, or by methods combining biological isolation and synthetic modification. By way of a non-limiting example, a nucleic acid could be isolated form from a biological source, or prepared by traditional techniques, known to those skilled in the art of nucleic acid synthesis. Furthermore, the nucleic acid may be further modified to contain non-naturally occurring moieties. Non-limiting examples of active biological agents include peptides, proteins, enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates and the like. They further include, but are not limited to, antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals and chemotherapeutic agents. Preferably, the active biological agent is a peptide, protein or enzyme, including derivatives and analogs of natural peptides, proteins and enzymes. The active biological agent may also be a hormone, gene therapies, RNA, siRNA, and/or cellular therapies (for non-limiting example, stem cells or T-cells).

“Active agent” as used herein refers to any pharmaceutical agent or active biological agent as described herein.

An “anti-cancer agent”, “anti-tumor agent” or “chemotherapeutic agent” refers to any agent useful in the treatment of a neoplastic condition. There are many chemotherapeutic agents available in commercial use, in clinical evaluation and in pre-clinical development that are useful in the devices and methods of the present invention for treatment of cancers.

In some embodiments, a chemotherapeutic agent comprises at least one of an angiostatin, DNA topoisomerase, endostatin, genistein, ornithine decarboxylase inhibitors, chlormethine, melphalan, pipobroman, triethylene-melamine, triethylenethiophosphoramine, busulfan, carmustine (BCNU), streptozocin, 6-mercaptopurine, 6-thioguanine, Deoxyco-formycin, IFN-α, 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, estramustine, medroxyprogesteroneacetate, flutamide, zoladex, mitotane, hexamethylmelamine, indolyl-3-glyoxylic acid derivatives, (e.g., indibulin), doxorubicin and idarubicin, plicamycin (mithramycin) and mitomycin, mechlorethamine, cyclophosphamide analogs, trazenes—dacarbazinine (DTIC), pentostatin and 2-chlorodeoxyadenosine, letrozole, camptothecin (and derivatives), navelbine, erlotinib, capecitabine, acivicin, acodazole hydrochloride, acronine, adozelesin, aldesleukin, ambomycin, ametantrone acetate, anthramycin, asperlin, azacitidine, azetepa, azotomycin, batimastat, benzodepa, bisnafide, bisnafide dimesylate, bizelesin, bropirimine, cactinomycin, calusterone, carbetimer, carubicin hydrochloride, carzelesin, cedefingol, celecoxib (COX-2 inhibitor), cirolemycin, crisnatol mesylate, decitabine, dexormaplatin, dezaguanine mesylate, diaziquone, duazomycin, edatrexate, eflomithine, elsamitrucin, enloplatin, enpromate, epipropidine, erbulozole, etanidazole, etoprine, fluorocitabine, fosquidone, lometrexol, losoxantrone hydrochloride, masoprocol, maytansine, megestrol acetate, melengestrol acetate, metoprine, meturedepa, mitindomide, mitocarcin, mitocromin, mitogillin, mitomalcin, mitosper, mycophenolic acid, nocodazole, nogalamycin, ormaplatin, oxisuran, pegaspargase, peliomycin, pentamustine, perfosfamide, piposulfan, plomestane, porfimer sodium, porfiromycin, puromycin, pyrazofurin, riboprine, safingol, simtrazene, sparfosate sodium, spiromustine, spiroplatin, streptonigrin, sulofenur, tecogalan sodium, taxotere, tegafur, teloxantrone hydrochloride, temoporfin, thiamiprine, tirapazamine, trestolone acetate, triciribine phosphate, trimetrexate glucuronate, tubulozole hydrochloride, uracil mustard, uredepa, verteporfin, vinepidine sulfate, vinglycinate sulfate, vinleurosine sulfate, vinorelbine tartrate, vinrosidine sulfate, zeniplatin, zinostatin, 20-epi-1,25 dihydroxyvitamin D3,5-ethynyluracil, acylfulvene, adecypenol, ALL-TK antagonists, ambamustine, amidox, amifostine, aminolevulinic acid, amrubicin, anagrelide, andrographolide, antagonist D, antagonist G, antarelix, anti-dorsalizing morphogenetic protein-1, antiandrogen, antiestrogen, estrogen agonist, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, BCR/ABL antagonists, benzochlorins, benzoylstaurosporine, beta lactam derivatives, beta-alethine, betaclamycin B, betulinic acid, bFGF inhibitor, bisaziridinylspermine, bistratene A, breflate, buthionine sulfoximine, calcipotriol, calphostin C, carboxamide-amino-triazole, carboxyamidotriazole, CaRest M3, CARN 700, cartilage derived inhibitor, casein kinase inhibitors (ICOS), castanospermine, cecropin B, cetrorelix, chloroquinoxaline sulfonamide, cicaprost, cis-porphyrin, clomifene analogues, clotrimazole, collismycin A, collismycin B, combretastatin A4, combretastatin analogue, conagenin, crambescidin 816, cryptophycin 8, cryptophycin A derivatives, curacin A, cyclopentanthraquinones, cycloplatam, cypemycin, cytolytic factor, cytostatin, dacliximab, dehydrodidemnin B, dexamethasone, dexifosfamide, dexrazoxane, dexverapamil, didemnin B, didox, diethylnorspermine, dihydro-5-azacytidine, dihydrotaxol, 9-, dioxamycin, docosanol, dolasetron, dronabinol, duocarmycin SA, ebselen, ecomustine, edelfosine, edrecolomab, elemene, emitefur, estramustine analogue, filgrastim, flavopiridol, flezelastine, fluasterone, fluorodaunorunicin hydrochloride, forfenimex, gadolinium texaphyrin, galocitabine, gelatinase inhibitors, glutathione inhibitors, hepsulfam, heregulin, hexamethylene bisacetamide, hypericin, ibandronic acid, idramantone, ilomastat, imatinib (e.g., Gleevec), imiquimod, immunostimulant peptides, insulin-like growth factor-1 receptor inhibitor, interferon agonists, interferons, interleukins, iobenguane, iododoxorubicin, ipomeanol, 4-, iroplact, irsogladine, isobengazole, isohomohalicondrin B, itasetron, jasplakinolide, kahalalide F, lamellarin-N triacetate, leinamycin, lenograstim, lentinan sulfate, leptolstatin, leukemia inhibiting factor, leukocyte alpha interferon, leuprolide+estrogen+progesterone, linear polyamine analogue, lipophilic disaccharide peptide, lipophilic platinum compounds, lissoclinamide 7, lobaplatin, lombricine, loxoribine, lurtotecan, lutetium texaphyrin, lysofylline, lytic peptides, maitansine, mannostatin A, marimastat, maspin, matrilysin inhibitors, matrix metalloproteinase inhibitors, meterelin, methioninase, metoclopramide, MIF inhibitor, mifepristone, miltefosine, mirimostim, mitoguazone, mitotoxin fibroblast growth factor-saporin, mofarotene, molgramostim, Erbitux, human chorionic gonadotrophin, monophosphoryl lipid A+myobacterium cell wall sk, mustard anticancer agent, mycaperoxide B, mycobacterial cell wall extract, myriaporone, N-acetyldinaline, N-substituted benzamides, nagrestip, naloxone+pentazocine, napavin, naphterpin, nartograstim, nedaplatin, nemorubicin, neridronic acid, nisamycin, nitric oxide modulators, nitroxide antioxidant, nitrullyn, oblimersen (Genasense), O⁶-benzylguanine, okicenone, onapristone, ondansetron, oracin, oral cytokine inducer, paclitaxel analogues and derivatives, palauamine, palmitoylrhizoxin, pamidronic acid, panaxytriol, panomifene, parabactin, peldesine, pentosan polysulfate sodium, pentrozole, perflubron, perillyl alcohol, phenazinomycin, phenylacetate, phosphatase inhibitors, picibanil, pilocarpine hydrochloride, placetin A, placetin B, plasminogen activator inhibitor, platinum complex, platinum compounds, platinum-triamine complex, propyl bis-acridone, prostaglandin J2, proteasome inhibitors, protein A-based immune modulator, protein kinase C inhibitors, microalgal, pyrazoloacridine, pyridoxylated hemoglobin polyoxyethylene conjugate, raf antagonists, raltitrexed, ramosetron, ras farnesyl protein transferase inhibitors, ras-GAP inhibitor, retelliptine demethylated, rhenium Re 186 etidronate, ribozymes, RH retinamide, rohitukine, romurtide, roquinimex, rubiginone B1, ruboxyl, saintopin, SarCNU, sarcophytol A, sargramostim, Sdi 1 mimetics, senescence derived inhibitor 1, signal transduction inhibitors, sizofuran, sobuzoxane, sodium borocaptate, solverol, somatomedin binding protein, sonermin, sparfosic acid, spicamycin D, splenopentin, spongistatin 1, squalamine, stipiamide, stromelysin inhibitors, sulfinosine, superactive vasoactive intestinal peptide antagonist, suradista, suramin, swainsonine, tallimustine, tazarotene, tellurapyrylium, telomerase inhibitors, tetrachlorodecaoxide, tetrazomine, thiocoraline, thrombopoietin, thrombopoietin mimetic, thymalfasin, thymopoietin receptor agonist, thymotrinan, thyroid stimulating hormone, tin ethyl etiopurpurin, titanocene bichloride, topsentin, translation inhibitors, tretinoin, triacetyluridine, tropisetron, turosteride, ubenimex, urogenital sinus-derived growth inhibitory factor, variolin B, velaresol, veramine, verdins, vinxaltine, vitaxin, zanoterone, zilascorb, zinostatin stimalamer, acanthifolic acid, aminothiadiazole, anastrozole, bicalutamide, brequinar sodium, capecitabine, carmofur, Ciba-Geigy CGP-30694, cladribine, cyclopentyl cytosine, cytarabine phosphate stearate, cytarabine conjugates, cytarabine ocfosfate, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine, fludarabine phosphate, N-(2′-furanidyl)-5-fluorouracil, Daiichi Seiyaku FO-152, 5-FU-fibrinogen, isopropyl pyrrolizine, Lilly LY-188011, Lilly LY-264618, methobenzaprim, methotrexate, Wellcome MZPES, norspermidine, nolvadex, NCI NSC-127716, NCI NSC-264880, NCI NSC-39661, NCI NSC-612567, Warner-Lambert PALA, pentostatin, piritrexim, plicamycin, Asahi Chemical PL-AC, stearate, Takeda TAC-788, thioguanine, tiazofurin, Erbamont TIF, trimetrexate, tyrosine kinase inhibitors, tyrosine protein kinase inhibitors, Taiho UFT, uricytin, Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)₂, diphenylspiromustine, diplatinum cytostatic, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, Ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromus-tine, Tanabe Seiyaku TA-077, tauromustine, temozolomide, teroxirone, tetraplatin and trimelamol, Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitomycin analogues, mitoxantrone, SmithKline M-TAG, neoenactin, Nippon Kayaku NK-313, Nippon Kayaku NKT-01, SR1 International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, zorubicin, 5-fluorouracil (5-FU), the peroxidate oxidation product of inosine, adenosine, or cytidine with methanol or ethanol, cytosine arabinoside (also referred to as Cytarabin, araC, and Cytosar), 5-Azacytidine, 2-Fluoroadenosine-5′-phosphate (Fludara, also referred to as FaraA), 2-Chlorodeoxyadenosine, Abarelix, Abbott A-84861, Abiraterone acetate, Aminoglutethimide, Asta Medica AN-207, Antide, Chugai AG-041R, Avorelin, aseranox, Sensus B2036-PEG, buserelin, BTG CB-7598, BTG CB-7630, Casodex, cetrolix, clastroban, clodronate disodium, Cosudex, Rotta Research CR-1505, cytadren, crinone, deslorelin, droloxifene, dutasteride, Elimina, Laval University EM-800, Laval University EM-652, epitiostanol, epristeride, Mediolanum EP-23904, EntreMed 2-ME, exemestane, fadrozole, finasteride, formestane, Pharmacia & Upjohn FCE-24304, ganirelix, goserelin, Shire gonadorelin agonist, Glaxo Wellcome GW-5638, Hoechst Marion Roussel Hoe-766, NCl hCG, idoxifene, isocordoin, Zeneca ICI-182780, Zeneca ICI-118630, Tulane University J015X, Schering Ag J96, ketanserin, lanreotide, Milkhaus LDI-200, letrozol, leuprolide, leuprorelin, liarozole, lisuride hydrogen maleate, loxiglumide, mepitiostane, Ligand Pharmaceuticals LG-1127, LG-1447, LG-2293, LG-2527, LG-2716, Bone Care International LR-103, Lilly LY-326315, Lilly LY-353381-HCl, Lilly LY-326391, Lilly LY-353381, Lilly LY-357489, miproxifene phosphate, Orion Pharma MPV-2213ad, Tulane University MZ-4-71, nafarelin, nilutamide, Snow Brand NKS01, Azko Nobel ORG-31710, Azko Nobel ORG-31806, orimeten, orimetene, orimetine, ormeloxifene, osaterone, Smithkline Beecham SKB-105657, Tokyo University OSW-1, Peptech PTL-03001, Pharmacia & Upjohn PNU-156765, quinagolide, ramorelix, Raloxifene, statin, sandostatin LAR, Shionogi S-10364, Novartis SMT-487, somavert, somatostatin, tamoxifen, tamoxifen methiodide, teverelix, toremifene, triptorelin, TT-232, vapreotide, vorozole, Yamanouchi YM-116, Yamanouchi YM-511, Yamanouchi YM-55208, Yamanouchi YM-53789, Schering AG ZK-1911703, Schering AG ZK-230211, and Zeneca ZD-182780, alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristo-Myers BMY-40481, Vestar boron-10, bromofosfamide, Wellcome BW-502, Wellcome BW-773, calcium carbonate, Calcet, Calci-Chew, Calci-Mix, Roxane calcium carbonate tablets, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Cell Pathways CP-461, Yakult Honsha CPT-11, crisnatol, curaderm, cytochalasin B, cytarabine, cytocytin, Merz D-609, DABIS maleate, datelliptinium, DFMO, didemnin-B, dihaematoporphyrin ether, dihydrolenperone dinaline, distamycin, Toyo Pharmar DM-341, Toyo Pharmar DM-75, Daiichi Seiyaku DN-9693, docetaxel, Encore Pharmaceuticals E7869, elliprabin, elliptinium acetate, Tsumura EPMTC, ergotamine, etoposide, etretinate, Eulexin, Cell Pathways Exisulind (sulindac sulphone or CP-246), fenretinide, Florical, Fujisawa FR-57704, gallium nitrate, gemcitabine, genkwadaphnin, Gerimed, Chugai GLA-43, Glaxo GR-63178, grifolan NMF-5N, hexadecylphosphocholine, Green Cross HO-221, homoharringtonine, hydroxyurea, BTG ICRF-187, ilmofosine, irinotecan, isoglutamine, isotretinoin, Otsuka JI-36, Ramot K-477, ketoconazole, Otsuak K-76COONa, Kureha Chemical K-AM, MECT Corp KI-8110, American Cyanamid L-623, leucovorin, levamisole, leukoregulin, lonidamine, Lundbeck LU-23-112, Lilly LY-186641, Materna, NCI (US) MAP, marycin, Merrel Dow MDL-27048, Medco MEDR-340, megestrol, merbarone, merocyanine derivatives, methylanilinoacridine, Molecular Genetics MGI-136, minactivin, mitonafide, mitoquidone, Monocal, mopidamol, motretinide, Zenyaku Kogyo MST-16, Mylanta, N-(retinoyl)amino acids, Nilandron, Nisshin Flour Milling N-021, N-acylated-dehydroalanines, nafazatrom, Taisho NCU-190, Nephro-Calci tablets, nocodazole derivative, Normosang, NCI NSC-145813, NCI NSC-361456, NCI NSC-604782, NCI NSC-95580, octreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, retinoids, R-flurbiprofen (Encore Pharmaceuticals), Sandostatin, Sapporo Breweries RBS, restrictin-P, retelliptine, retinoic acid, Rhone-Poulenc RP-49532, Rhone-Poulenc RP-56976, Scherring-Plough SC-57050, Scherring-Plough SC-57068, selenium (selenite and selenomethionine), SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, Sugen SU-101, Sugen SU-5416, Sugen SU-6668, sulindac, sulindac sulfone, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine, vinblastine sulfate, vincristine, vincristine sulfate, vindesine, vindesine sulfate, vinestramide, vinorelbine, vintriptol, vinzolidine, withanolides, Yamanouchi YM-534, Zileuton, ursodeoxycholic acid, Zanosar.

Chemotherapeutic agents and dosing recommendations for treating specific diseases, are described at length in the literature, e.g., in U.S. Pat. No. 6,858,598, “Method of Using a Matrix Metalloproteinase Inhibitor and One or More Antineoplastic Agents as a Combination Therapy in the Treatment of Neoplasia,” and U.S. Pat. No. 6,916,800, “Combination Therapy Including a Matrix Metalloproteinase Inhibitor and an Antineoplastic Agent,” both incorporated herein by reference in their entirety.

Methods for the safe and effective administration of chemotherapeutic agents are known to those skilled in the art. In addition, their administration is described in the standard literature. For example, the administration of many chemotherapeutic agents is described in the “Physicians' Desk Reference” (PDR), e.g., 1996 edition (Medical Economics Company, Montvale, N.J. 07645-1742, USA), incorporated herein by reference.

Combinations of two or more agents can be used in the devices and methods of the invention. Guidance for selecting drug combinations for given indications is provided in the published literature, e.g., in the “Drug Information Handbook for Oncology: A Complete Guide to Combination Chemotherapy Regimens” (edited by Dominic A. Solimando, Jr., MA BCOP; published by Lexi-Comp, Hudson, Ohio, 2007. ISBN 978-1-59195-175-9), as well as in U.S. Pat. No. 6,858,598. Specific combinations of chemotherapeutic agents having enhanced activity relative to the individual agents, are described in, e.g., WO 02/40702, “Methods for the Treatment of Cancer and Other Diseases and Methods of Developing the Same,” incorporated herein by reference in its entirety. WO 02/40702 reports enhanced activity when treating cancer using a combination of a platin-based compound (e.g., cisplatin, oxoplatin), a folate inhibitor (e.g., MTA, ALIMTA, LY231514), and deoxycytidine or an analogue thereof (e.g., cytarabin, gemcitabine).

Chemotherapeutic agents can be classified into various groups, e.g., ACE inhibitors, alkylating agents, angiogenesis inhibitors, anthracyclines/DNA intercalators, anti-cancer antibiotics or antibiotic-type agents, antimetabolites, antimetastatic compounds, asparaginases, bisphosphonates, cGMP phosphodiesterase inhibitors, cyclooxygenase-2 inhibitors DHA derivatives, epipodophylotoxins, hormonal anticancer agents, hydrophilic bile acids (URSO), immunomodulators or immunological agents, integrin antagonists, interferon antagonists or agents, MMP inhibitors, monoclonal antibodies, nitrosoureas, NSAIDs, ornithine decarboxylase inhibitors, radio/chemo sensitizers/protectors, retinoids, selective inhibitors of proliferation and migration of endothelial cells, selenium, stromelysin inhibitors, taxanes, vaccines, and vinca alkaloids.

Alternatively, chemotherapeutic agents can be classified by target, e.g., agents can be selected from a tubulin binding agent, a kinase inhibitor (e.g., a receptor tyrosine kinase inhibitor), an anti-metabolic agent, a DNA synthesis inhibitor, and a DNA damaging agent.

Other classes into which chemotherapeutic agents can be divided include: alkylating agents, antimetabolites, natural products and their derivatives, hormones and steroids (including synthetic analogs), and synthetics. Examples of compounds within these classes are given herein.

Alkylating agents (e.g., nitrogen mustards, ethylenimine derivatives, alkyl sulfonates, nitrosoureas and triazenes) include Uracil mustard, Chlormethine, Cyclophosphamide (Cytoxan), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (e.g., folic acid antagonists, pyrimidine analogs, purine analogs and adenosine deaminase inhibitors) include Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (e.g., vinca alkaloids, antitumor antibiotics, enzymes, lymphokines and epipodophyllotoxins) include Vinblastine, Vincristine, Vindesine, Bleomycin, Dactinomycin, Daunorubicin, Doxorubicin, Epirubicin, Idarubicin, paclitaxel (paclitaxel is commercially available as Taxol), Mithramycin, Deoxyco-formycin, Mitomycin-C, L-Asparaginase, Interferons (especially IFN-α), Etoposide, and Teniposide.

Hormones and steroids (e.g., synthetic analogs) include 17α-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Zoladex.

Synthetics (e.g., inorganic complexes such as platinum coordination complexes) include Cisplatin, Carboplatin, Hydroxyurea, Amsacrine, Procarbazine, Mitotane, Mitoxantrone, Levamisole, and Hexamethylmelamine.

Chemotherapeutic agents can also be classified by chemical family, for example, therapeutic agents selected from vinca alkaloids (e.g., vinblastine, vincristine, and vinorelbine), taxanes (e.g., paclitaxel and docetaxel), indolyl-3-glyoxylic acid derivatives, (e.g., indibulin), epidipodophyllotoxins (e.g., etoposide, teniposide), antibiotics (e.g., dactinomycin or actinomycin D, daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (e.g., mechlorethamine, ifosphamide, cyclophosphamide and analogs, melphalan, chlorambucil), ethylenimines and methylmelamines (e.g., hexamethylmelamine and thiotepa), alkyl sulfonates (busulfan), nitrosoureas (e.g., carmustine (BCNU) and analogs, streptozocin), trazenes—dacarbazinine (DTIC); antiproliferative/antimitotic antimetabolites such as folic acid analogs (e.g., methotrexate), pyrimidine analogs (e.g., fluorouracil, floxuridine, and cytarabine), purine analogs and related inhibitors (e.g., mercaptopurine, thioguanine, pentostatin and 2-chlorodeoxyadenosine); aromatase inhibitors (e.g., anastrozole, exemestane, and letrozole); and platinum coordination complexes (e.g., cisplatin, carboplatin), procarbazine, hydroxyurea, mitotane, aminoglutethimide; hormones (e.g., estrogen) and hormone agonists such as leutinizing hormone releasing hormone (LHRH) agonists (e.g., goserelin, leuprolide and triptorelin).

Antineoplastic agents are often placed into categories, including antimetabolite agents, alkylating agents, antibiotic-type agents, hormonal anticancer agents, immunological agents, interferon-type agents, and a category of miscellaneous antineoplastic agents. Some antineoplastic agents operate through multiple or unknown mechanisms and can thus be classified into more than one category.

A first family of antineoplastic agents which may be used in combination with the present invention consists of antimetabolite-type antineoplastic agents. Antimetabolites are typically reversible or irreversible enzyme inhibitors, or compounds that otherwise interfere with the replication, translation or transcription of nucleic acids. Suitable antimetabolite antineoplastic agents that may be used in the present invention include, but are not limited to acanthifolic acid, aminothiadiazole, anastrozole, bicalutamide, brequinar sodium, capecitabine, carmofur, Ciba-Geigy CGP-30694, cladribine, cyclopentyl cytosine, cytarabine phosphate stearate, cytarabine conjugates, cytarabine ocfosfate, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, finasteride, floxuridine, fludarabine, fludarabine phosphate, N-(2′-furanidyl)-5-fluorouracil, Daiichi Seiyaku FO-152, fluorouracil (5-FU), 5-FU-fibrinogen, isopropyl pyrrolizine, Lilly LY-188011, Lilly LY-264618, methobenzaprim, methotrexate, Wellcome MZPES, nafarelin, norspermidine, nolvadex, NCI NSC-127716, NCI NSC-264880, NCI NSC-39661, NCI NSC-612567, Warner-Lambert PALA, pentostatin, piritrexim, plicamycin, Asahi Chemical PL-AC, stearate; Takeda TAC-788, thioguanine, tiazofurin, Erbamont TIF, trimetrexate, tyrosine kinase inhibitors, tyrosine protein kinase inhibitors, Taiho UFT, toremifene, and uricytin.

Antimetabolite agents that may be used in the present invention include, but are not limited to, those identified in Table No. 5 of U.S. Pat. No. 6,858,598, which is incorporated herein by reference.

A second family of antineoplastic agents which may be used in combination with the present invention consists of alkylating-type antineoplastic agents. The alkylating agents are believed to act by alkylating and cross-linking guanine and possibly other bases in DNA, arresting cell division. Typical alkylating agents include nitrogen mustards, ethyleneimine compounds, alkyl sulfates, cisplatin, and various nitrosoureas. A disadvantage with these compounds is that they not only attack malignant cells, but also other cells which are naturally dividing, such as those of bone marrow, skin, gastro-intestinal mucosa, and fetal tissue. Suitable alkylating-type antineoplastic agents that may be used in the present invention include, but are not limited to, Shionogi 254-S, aldo-phosphamide analogues, altretamine, anaxirone, Boehringer Mannheim BBR-2207, bestrabucil, budotitane, Wakunaga CA-102, carboplatin, carmustine (BiCNU), Chinoin-139, Chinoin-153, chlorambucil, cisplatin, cyclophosphamide, American Cyanamid CL-286558, Sanofi CY-233, cyplatate, dacarbazine, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, etoposide phosphate, fotemustine, Unimed G-6-M, Chinoin GYKI-17230, hepsul-fam, ifosfamide, iproplatin, lomustine, mafosfamide, mitolactol, mycophenolate, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, thiotepa, Yakult Honsha SN-22, spiromus-tine, Tanabe Seiyaku TA-077, tauromustine, temozolomide, teroxirone, tetraplatin and trimelamol.

Preferred alkylating agents that may be used in the present invention include, but are not limited to, those identified in Table No. 6 of U.S. Pat. No. 6,858,598, which is incorporated herein by reference.

A third family of antineoplastic agents which may be used in combination with the present invention consists of antibiotic-type antineoplastic agents. Suitable antibiotic-type antineoplastic agents that may be used in the present invention include, but are not limited to Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 and zorubicin.

Preferred antibiotic anticancer agents that may be used in the present invention include, but are not limited to, those identified in Table No. 7 of U.S. Pat. No. 6,858,598, which is incorporated herein by reference.

A fourth family of antineoplastic agents which may be used in combination with the present invention consists of synthetic nucleosides. Several synthetic nucleosides have been identified that exhibit anticancer activity. A well known nucleoside derivative with strong anticancer activity is 5-fluorouracil (5-FU). 5-Fluorouracil has been used clinically in the treatment of malignant tumors, including, for example, carcinomas, sarcomas, skin cancer, cancer of the digestive organs, and breast cancer. 5-Fluorouracil, however, causes serious adverse reactions such as nausea, alopecia, diarrhea, stomatitis, leukocytic thrombocytopenia, anorexia, pigmentation, and edema. Derivatives of 5-fluorouracil with anti-cancer activity have been described in U.S. Pat. No. 4,336,381. Further 5-FU derivatives have been described in the following patents identified in Table No. 8 of U.S. Pat. No. 6,858,598, which is incorporated herein by reference.

U.S. Pat. No. 4,000,137 discloses that the peroxidate oxidation product of inosine, adenosine, or cytidine with methanol or ethanol has activity against lymphocytic leukemia. Cytosine arabinoside (also referred to as Cytarabin, araC, and Cytosar) is a nucleoside analog of deoxycytidine that was first synthesized in 1950 and introduced into clinical medicine in 1963. It is currently an important drug in the treatment of acute myeloid leukemia. It is also active against acute lymphocytic leukemia, and to a lesser extent, is useful in chronic myelocytic leukemia and non-Hodgkin's lymphoma. The primary action of araC is inhibition of nuclear DNA synthesis. Handschumacher, R. and Cheng, Y., “Purine and Pyrimidine Antimetabolites”, Cancer Medicine, Chapter XV-1, 3rd Edition, Edited by J. Holland, et al., Lea and Febigol, publishers.

5-Azacytidine is a cytidine analog that is primarily used in the treatment of acute myelocytic leukemia and myelodysplastic syndrome.

2-Fluoroadenosine-5′-phosphate (Fludara, also referred to as FaraA) is one of the most active agents in the treatment of chronic lymphocytic leukemia. The compound acts by inhibiting DNA synthesis. Treatment of cells with F-araA is associated with the accumulation of cells at the G1/S phase boundary and in S phase; thus, it is a cell cycle S phase-specific drug. InCorp of the active metabolite, F-araATP, retards DNA chain elongation. F-araA is also a potent inhibitor of ribonucleotide reductase, the key enzyme responsible for the formation of dATP. 2-Chlorodeoxyadenosine is useful in the treatment of low grade B-cell neoplasms such as chronic lymphocytic leukemia, non-Hodgkins' lymphoma, and hairy-cell leukemia. The spectrum of activity is similar to that of Fludara. The compound inhibits DNA synthesis in growing cells and inhibits DNA repair in resting cells.

A fifth family of antineoplastic agents which may be used in combination with the present invention consists of hormonal agents. Suitable hormonal-type antineoplastic agents that may be used in the present invention include, but are not limited to Abarelix; Abbott A-84861; Abiraterone acetate; Aminoglutethimide; anastrozole; Asta Medica AN-207; Antide; Chugai AG-041R; Avorelin; aseranox; Sensus B2036-PEG; Bicalutamide; buserelin; BTG CB-7598, BTG CB-7630; Casodex; cetrolix; clastroban; clodronate disodium; Cosudex; Rotta Research CR-1505; cytadren; crinone; deslorelin; droloxifene; dutasteride; Elimina; Laval University EM-800; Laval University EM-652; epitiostanol; epristeride; Mediolanum EP-23904; EntreMed 2-ME; exemestane; fadrozole; finasteride; flutamide; formestane; Pharmacia & Upjohn FCE-24304; ganirelix; goserelin; Shire gonadorelin agonist; Glaxo Wellcome GW-5638; Hoechst Marion Roussel Hoe-766; NCl hCG; idoxifene; isocordoin; Zeneca ICI-182780; Zeneca ICI-118630; Tulane University J015X; Schering Ag J96; ketanserin; lanreotide; Milkhaus LDI-200; letrozol; leuprolide; leuprorelin; liarozole; lisuride hydrogen maleate; loxiglumide; mepitiostane; Leuprorelin; Ligand Pharmaceuticals LG-1127; LG-1447; LG-2293; LG-2527; LG-2716; Bone Care International LR-103; Lilly LY-326315; Lilly LY-353381-HCl; Lilly LY-326391; Lilly LY-353381; Lilly LY-357489; miproxifene phosphate; Orion Pharma MPV-2213ad; Tulane University MZ-4-71; nafarelin; nilutamide; Snow Brand NKS01; octreotide; Azko Nobel ORG-31710; Azko Nobel ORG-31806; orimeten; orimetene; orimetine; ormeloxifene; osaterone; Smithkline Beecham SKB-105657; Tokyo University OSW-1; Peptech PTL-03001; Pharmacia & Upjohn PNU-156765; quinagolide; ramorelix; Raloxifene; statin; sandostatin LAR; Shionogi S-10364; Novartis SMT-487; somavert; somatostatin; tamoxifen; tamoxifen methiodide; teverelix; toremifene; triptorelin; TT-232; vapreotide; vorozole; Yamanouchi YM-116; Yamanouchi YM-511; Yamanouchi YM-55208; Yamanouchi YM-53789; Schering AG ZK-1911703; Schering AG ZK-230211; and Zeneca ZD-182780.

Preferred hormonal agents that may be used in the present invention include, but are not limited to, those identified in Table No. 9 of U.S. Pat. No. 6,858,598, which is incorporated herein by reference.

A sixth family of antineoplastic agents which may be used in combination with the present invention consists of a miscellaneous family of antineoplastic agents including, but not limited to alpha-carotene, alpha-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristo-Myers BMY-40481, Vestar boron-10, bromofosfamide, Wellcome BW-502, Wellcome BW-773, calcium carbonate, Calcet, Calci-Chew, Calci-Mix, Roxane calcium carbonate tablets, caracemide, carmethizole hydrochloride, Ajinomoto CDAF, chlorsulfaquinoxalone, Chemes CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Cell Pathways CP-461, Yakult Honsha CPT-11, crisnatol, curaderm, cytochalasin B, cytarabine, cytocytin, Merz D-609, DABIS maleate, dacarbazine, datelliptinium, DFMO, didemnin-B, dihaematoporphyrin ether, dihydrolenperone dinaline, distamycin, Toyo Pharmar DM-341, Toyo Pharmar DM-75, Daiichi Seiyaku DN-9693, docetaxel, Encore Pharmaceuticals E7869, elliprabin, elliptinium acetate, Tsumura EPMTC, ergotamine, etoposide, etretinate, Eulexin, Cell Pathways Exisulind (sulindac sulphone or CP-246), fenretinide, Merck Research Labs Finasteride, Florical, Fujisawa FR-57704, gallium nitrate, gemcitabine, genkwadaphnin, Gerimed, Chugai GLA-43, Glaxo GR-63178, grifolan NMF-5N, hexadecylphosphocholine, Green Cross HO-221, homoharringtonine, hydroxyurea, BTG ICRF-187, ilmofosine, irinotecan, isoglutamine, isotretinoin, Otsuka JI-36, Ramot K-477, ketoconazole, Otsuak K-76COONa, Kureha Chemical K-AM, MECT Corp KI-8110, American Cyanamid L-623, leucovorin, levamisole, leukoregulin, lonidamine, Lundbeck LU-23-112, Lilly LY-186641, Materna, NCl (US) MAP, marycin, Merrel Dow MDL-27048, Medco MEDR-340, megestrol, merbarone, merocyanine derivatives, methylanilinoacridine, Molecular Genetics MGI-136, minactivin, mitonafide, mitoquidone, Monocal, mopidamol, motretinide, Zenyaku Kogyo MST-16, Mylanta, N-(retinoyl)amino acids, Nilandron; Nisshin Flour Milling N-021, N-acylated-dehydroalanines, nafazatrom, Taisho NCU-190, Nephro-Calci tablets, nocodazole derivative, Normosang, NCI NSC-145813, NCI NSC-361456, NCI NSC-604782, NCI NSC-95580, octreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, retinoids, R-flurbiprofen (Encore Pharmaceuticals), Sandostatin; Sapporo Breweries RBS, restrictin-P, retelliptine, retinoic acid, Rhone-Poulenc RP-49532, Rhone-Poulenc RP-56976, Scherring-Plough SC-57050, Scherring-Plough SC-57068, selenium (selenite and selenomethionine), SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, Sugen SU-101, Sugen SU-5416, Sugen SU-6668, sulindac, sulindac sulfone; superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, withanolides, Yamanouchi YM-534, Zileuton, ursodeoxycholic acid, and Zanosar.

Preferred miscellaneous agents that may be used in the present invention include, but are not limited to, those identified in (the second) Table No. 6 of U.S. Pat. No. 6,858,598, which is incorporated herein by reference.

Some additional preferred antineoplastic agents include those described in the individual patents listed in U.S. Pat. No. 6,858,598 in (the second) Table No. 7, and are hereby individually incorporated by reference.

“Activity” as used herein refers to the ability of a pharmaceutical or active biological agent to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). Thus the activity of a pharmaceutical or active biological agent should be of therapeutic or prophylactic value.

“Secondary, tertiary and quaternary structure” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The α-helix and the β-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two α and two β chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

An “antibiotic agent,” as used herein, is a substance or compound that kills bacteria (i.e., is bacteriocidal) or inhibits the growth of bacteria (i.e., is bacteriostatic).

Antibiotics that can be used in the devices and methods of the present invention include, but are not limited to, amikacin, amoxicillin, gentamicin, kanamycin, neomycin, netilmicin, paromomycin, tobramycin, geldanamycin, herbimycin, carbacephem (loracarbef), ertapenem, doripenem, imipenem, cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, clarithromycin, clavulanic acid, clindamycin, teicoplanin, azithromycin, dirithromycin, erythromycin, troleandomycin, telithromycin, aztreonam, ampicillin, azlocillin, bacampicillin, carbenicillin, cloxacillin, dicloxacillin, flucloxacillin, mezlocillin, meticillin, nafcillin, norfloxacin, oxacillin, penicillin G, penicillin V, piperacillin, pvampicillin, pivmecillinam, ticarcillin, bacitracin, colistin, polymyxin B, ciprofloxacin, enoxacin, gatifloxacin, levofloxacin, lomefloxacin, moxifloxacin, ofloxacin, trovafloxacin, grepafloxacin, sparfloxacin, afenide, prontosil, sulfacetamide, sulfamethizole, sulfanilimide, sulfamethoxazole, sulfisoxazole, trimethoprim, trimethoprim-sulfamethoxazole, demeclocycline, doxycycline, oxytetracycline, tetracycline, arsphenamine, chloramphenicol, lincomycin, ethambutol, fosfomycin, furazolidone, isoniazid, linezolid, mupirocin, nitrofurantoin, platensimycin, pyrazinamide, quinupristin/dalfopristin, rifampin, thiamphenicol, rifampicin, minocycline, sultamicillin, sulbactam, sulphonamides, mitomycin, spectinomycin, spiramycin, roxithromycin, and meropenem.

Antibiotics can also be grouped into classes of related drugs, for example, aminoglycosides (e.g., amikacin, gentamicin, kanamycin, neomycin, netilmicin, paromomycin, streptomycin, tobramycin), ansamycins (e.g., geldanamycin, herbimycin), carbacephem (loracarbef) carbapenems (e.g., ertapenem, doripenem, imipenem, meropenem), first generation cephalosporins (e.g., cefadroxil, cefazolin, cefalotin, cefalexin), second generation cephalosporins (e.g., cefaclor, cefamandole, cefoxitin, cefprozil, cefuroxime), third generation cephalosporins (e.g., cefixime, cefdinir, cefditoren, cefoperazone, cefotaxime, cefpodoxime, ceftazidime, ceftibuten, ceftizoxime, ceftriaxone), fourth generation cephalosporins (e.g., cefepime), fifth generation cephalosporins (e.g., ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, bacampicillin, carbenicillin, cloxacillin, dicloxacillin, flucloxacillin, mezlocillin, meticillin, nafcillin, oxacillin, penicillins G and V, piperacillin, pvampicillin, pivmecillinam, ticarcillin), polypeptides (e.g., bacitracin, colistin, polymyxin B), quinolones (e.g., ciprofloxacin, enoxacin, gatifloxacin, levofloxacin, lomefloxacin, moxifloxacin, norfloxacin, ofloxacin, trovafloxacin, grepafloxacin, sparfloxacin, trovafloxacin), sulfonamides (e.g., afenide, pronto sil, sulfacetamide, sulfamethizole, sulfanilimide, sulfasalazine, sulfamethoxazole, sulfisoxazole, trimethoprim, trimethoprim-sulfamethoxazole), tetracyclines (e.g., demeclocycline, doxycycline, minocycline, oxytetracycline, tetracycline).

For treatment of abcesses, commonly caused by Staphylococcus aureus bacteria, use of an anti-staphylococcus antibiotic such as flucloxacillin or dicloxacillin is contemplated. With the emergence of community-acquired methicillin-resistant staphylococcus aureus MRSA, these traditional antibiotics may be ineffective; alternative antibiotics effective against community-acquired MRSA often include clindamycin, trimethoprim-sulfamethoxazole, and doxycycline. These antibiotics may also be prescribed to patients with a documented allergy to penicillin. If the condition is thought to be cellulitis rather than abscess, consideration should be given to possibility of strep species as cause that are still sensitive to traditional anti-staphylococcus agents such as dicloxacillin or cephalexin in patients able to tolerate penicillin.

Anti-thrombotic agents are contemplated for use in the methods of the invention in adjunctive therapy for treatment of coronary stenosis. The use of anti-platelet drugs, e.g., to prevent platelet binding to exposed collagen, is contemplated for anti-restenotic or anti-thrombotic therapy. Anti-platelet agents include “GpIIb/IIIa inhibitors” (e.g., abciximab, eptifibatide, tirofiban, RheoPro) and “ADP receptor blockers” (prasugrel, clopidogrel, ticlopidine). Particularly useful for local therapy are dipyridamole, which has local vascular effects that improve endothelial function (e.g., by causing local release of t-PA, that will break up clots or prevent clot formation) and reduce the likelihood of platelets and inflammatory cells binding to damaged endothelium, and cAMP phosphodiesterase inhibitors, e.g., cilostazol, that could bind to receptors on either injured endothelial cells or bound and injured platelets to prevent further platelet binding.

The methods of the invention are useful for encouraging migration and proliferation of endothelial cells from adjacent vascular domains to “heal” the damaged endothelium and/or encourage homing and maturation of blood-borne endothelial progenitor cells to the site of injury. There is evidence that both rapamycin and paclitaxel prevent endothelial cell growth and reduce the colonization and maturation of endothelial progenitor cells (EPCs) making both drugs ‘anti-healing.’ While local delivery of growth factors could accelerate endothelial cell regrowth, virtually all of these agents are equally effective at accelerating the proliferation of vascular smooth muscle cells, which can cause restenosis. VEGF is also not selective for endothelial cells but can cause proliferation of smooth muscle cells. To make VEGF more selective for endothelial cells it can be combined with a proteoglycan like heparan sulfate or chondroitin sulfate or even with an elongated “RGD” peptide binding domain. This may sequester it away from the actual lesion site but still allow it to dissociate and interact with nearby endothelial cells. The use of CD34 antibodies and other specific antibodies, which bind to the surface of blood borne progenitor cells, can be used to attract endothelial progenitor cells to the vessel wall to potential accelerate endothelialization.

Statins (e.g., cerivastatin, etorvastatin), which can have endothelial protective effects and improve progenitor cell function, are contemplated for use in embodiments of methods and/or devices provided herein. Other drugs that have demonstrated some evidence to improve EPC colonization, maturation or function and are contemplated for use in the methods of the invention are angiotensin converting enzyme inhibitors (ACE-I, e.g., Captopril, Enalapril, and Ramipril), Angiotensin II type I receptor blockers (AT-II-blockers, e.g., losartan, valartan), peroxisome proliferator-activated receptor gamma (PPAR-γ) agonists, and erythropoietin. The PPAR-γ agonists like the glitazones (e.g., rosiglitazone, pioglitazone) can provide useful vascular effects, including the ability to inhibit vascular smooth muscle cell proliferation, and have anti-inflammatory functions, local antithrombotic properties, local lipid lowing effects, and can inhibit matrix metalloproteinase (MMP) activity so as to stabilize vulnerable plaque.

Atherosclerosis is viewed as a systemic disease with significant local events. Adjunctive local therapy can be used in addition to systemic therapy to treat particularly vulnerable areas of the vascular anatomy. The mutant protein Apo A1 Milano has been reported to remove unwanted lipid from a blood vessel and can cause regression of atherosclerosis. Either protein therapy, or gene therapy to provide sustained release of a protein therapy, can be delivered using the methods of the invention. Adiponectin, a protein produced by adipocytes, is another protein with anti-atherosclerotic properties. It prevents inflammatory cell binding and promotes generation of nitric oxide (NO). NO has been shown to have antiatherogenic activity in the vessel wall; it promotes antiinflammatory and other beneficial effects. The use of agents including nitric oxide synthase (NOS) gene therapy that act to increase NO levels, are contemplated herein. NOS gene therapy is described, e.g., by Channon, et al., 2000, “Nitric Oxide Synthase in Atherosclerosis and Vascular Injury: Insights from Experimental Gene Therapy,” Arteriosclerosis, Thrombosis, and Vascular Biology, 20 (8):1873-1881. Compounds for treating NO deficiency are described, e.g., in U.S. Pat. No. 7,537,785, “Composition for treating vascular diseases characterized by nitric oxide insufficiency,” incorporated herein by reference in its entirety. “Vulnerable plaque” occurs in blood vessels where a pool of lipid lies below a thin fibrous cap. If the cap ruptures then the highly thrombogenic lipid leaks into the artery often resulting in abrupt closure of the vessel due to rapid clotting. Depending on the location of the vulnerable plaque, rupture can lead to sudden death. Both statins and glitazones have been shown to strengthen the fibrous cap covering the plaque and make it less vulnerable. Other agents, e.g., batimastat or marimastat, target the MMPs that can destroy the fibrin cap.

Angiogenesis promoters can be used for treating reperfusion injury, which can occur when severely stenotic arteries, particular chronic total occlusions, are opened. Angiogenesis promoters are contemplated for use in embodiments of methods and/or devices provided herein. Myocardial cells downstream from a blocked artery will downregulate the pathways normally used to prevent damage from oxygen free radicals and other blood borne toxins. A sudden infusion of oxygen can lead to irreversible cell damage and death. Drugs developed to prevent this phenomenon can be effective if provided by sustained local delivery. Neurovascular interventions can particularly benefit from this treatment strategy. Examples of pharmacological agents potentially useful in preventing reperfusion injury are glucagon-like peptide 1, erythropoietin, atorvastatin, and atrial natriuretic peptide (ANP). Other angiogenesis promoters have been described, e.g., in U.S. Pat. No. 6,284,758, “Angiogenesis promoters and angiogenesis potentiators,” U.S. Pat. No. 7,462,593, “Compositions and methods for promoting angiogenesis,” and U.S. Pat. No. 7,456,151, “Promoting angiogenesis with netrinl polypeptides.”

“Local anesthetics” are substances which inhibit pain signals in a localized region. Examples of such anesthetics include procaine, lidocaine, tetracaine and dibucaine. Local anesthetics are contemplated for use in embodiments of methods and/or devices provided herein.

“Anti-inflammatory agents” as used herein refer to agents used to reduce inflammation. Anti-inflammatory agents useful in the devices and methods of the invention include, but are not limited to: aspirin, ibuprofen, naproxen, hyssop, ginger, turmeric, helenalin, cannabichromene, rofecoxib, celecoxib, paracetamol (acetaminophen), sirolimus (rapamycin), dexamethasone, dipyridamole, alfuzosin, statins, and glitazones. Antiinflammatory agents are contemplated for use in embodiments of methods and/or devices provided herein.

Antiinflammatory agents can be classified by action. For example, glucocorticoids are steroids that reduce inflammation or swelling by binding to cortisol receptors. Non-steroidal anti-inflammatory drugs (NSAIDs), alleviate pain by acting on the cyclooxygenase (COX) enzyme. COX synthesizes prostaglandins, causing inflammation. A cannabinoid, cannabichromene, present in the cannabis plant, has been reported to reduce inflammation. Newer COX-inhibitors, e.g., rofecoxib and celecoxib, are also antiinflammatory agents. Many antiinflammatory agents are also analgesics (painkillers), including salicylic acid, paracetamol (acetaminophen), COX-2 inhibitors and NSAIDs. Also included among analgesics are, e.g., narcotic drugs such as morphine, and synthetic drugs with narcotic properties such as tramadol.

Other antiinflammatory agents useful in the methods of the present invention include sirolimus (rapamycin) and dexamethasone. S tents coated with dexamethasone were reported to be useful in a particular subset of patients with exaggerated inflammatory disease evidenced by high plasma C-reactive protein levels. Because both restenosis and atherosclerosis have such a large inflammatory component, anti-inflammatories remain of interest with regard to local therapeutic agents. In particular, the use of agents that have anti-inflammatory activity in addition to other useful pharmacologic actions is contemplated. Examples include dipyridamole, statins and glitazones. Despite an increase in cardiovascular risk and systemic adverse events reported with use of cyclooxygenase (COX)-inhibitors (e.g., celocoxib), these drugs can be useful for short term local therapy.

It is understood that certain agents will fall into multiple categories of agents, for example, certain antibiotic agents are also chemotherapeutic agents, and biological agents can include antibiotic agents, etc.

Specific pharmaceutical agents useful in certain embodiments of devices and/or methods of the invention are hyaluronidases. Hylenex (Baxter International, Inc.) is a formulation of a human recombinant hyaluronidase, PH-20, that is used to facilitate the absorption and dispersion of other injected drugs or fluids. When injected under the skin or in the muscle, hyaluronidase can digest the hyaluronic acid gel, allowing for temporarily enhanced penetration and dispersion of other injected drugs or fluids.

Hyaluronidase can allow drugs to pass more freely to target tissues. It has been observed on its own to suppress tumor growth, and is thus a chemotherapeutic agent. For example, increased drug antitumor activity has been reported by Halozyme Therapeutics (Carlsbad, Calif.), when hyaluronidase is used in conjunction with another chemotherapeutic agent to treat an HA-producing tumor (reports available at http://www.halozyme.com). A pegylated hyaluronidase product (PEGPH₂O) is currently being tested as a treatment for prostate cancer, and a product containing both hyaluronidase and mitomycin C (Chemophase) is being tested for treatment of bladder cancer.

In certain embodiments of devices and/or methods provided herein, hyaluronidase is used for treating any HA-producing cancer, either alone or in combination with another chemotherapeutic agent. In particular embodiments, hyaluronidase is used in the methods of the invention for treating bladder cancer, e.g., in combination with mitomycin C. In other embodiments, hyaluronidase is used for treating prostate cancer. Cancers potentially treated with hyaluronidase include, but are not limited to, Kaposi's sarcoma, glioma, melanocyte, head and neck squamous cell carcinoma, breast cancer, gastrointestinal cancer, and other genitourinary cancers, e.g., testicular cancer and ovarian cancer. The correlation of HA with various cancers has been described in the literature, e.g., by Simpson, et al., Front Biosci. 13:5664-5680. In embodiments, hyaluronidase is used in the devices and methods of the invention to enhance penetration and dispersion of any agents described herein, including, e.g., painkillers, antiinflammatory agents, etc., in particular, to tissues that produce HA.

Hyaluronidases are described, e.g., in U.S. Pat. App. No. 2005/0260186 and 2006/0104968, both titled “Soluble glycosaminoglycanases and methods of preparing and using soluble glycosaminoglycanases” and incorporated herein by reference in their entirety. Bookbinder, et al., 2006, “A recombinant human enzyme for enhanced interstitial transport of therapeutics,” Journal of Controlled Release 114:230-241 reported improved pharmacokinetic profile and absolute bioavailability, of peginterferon alpha-2b or the antiinflammatory agent infliximab, when either one is coinjected with rHuPH2O (human recombinant hyaluronidase PH-20). They also reported that an increased volume of drug could be injected subcutaneously when coinjected with hyaluronidase. Methods for providing human plasma hyaluronidases, and assays for hyaluronidases, are described in, e.g., U.S. Pat. No. 7,148,201, “Use of human plasma hyaluronidase in cancer treatment,” incorporated herein by reference in its entirety. The use of hyaluronidase in the devices and methods of the invention is expected to increase the rate and amount of drug absorbed, providing an added aspect to control over release rates.

Hyaluronidase co-delivery is also useful when an agent is administered using the devices and methods of the invention within a tissue not having a well-defined preexisting cavity or having a cavity that is smaller than the inflated delivery balloon. In these embodiments, inflation of the delivery balloon creates a cavity where either none existed or greatly enlarges an existing cavity. For example, a solid tumor can be treated with hyaluronidase and a chemotherapeutic agent using a delivery balloon inserted through, e.g., a biopsy needle or the like. Vasoactive agents, e.g., TNF-alpha and histamine, also can be used to improve drug distribution within the tumor tissue. (See, e.g., Brunstein, et al., 2006, “Histamine, a vasoactive agent with vascular disrupting potential improves tumour response by enhancing local drug delivery,” British Journal of Cancer 95:1663-1669). As another example of treatment of a location lacking a preexisting cavity, dense muscle tissue can be treated locally with a slow-release painkiller, using a delivery balloon inserted through a hollow needle.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments, of the invention only one polymer is used. In some preferred embodiments a combination of two polymers are used. Combinations of polymers can be in varying ratios, to provide coatings with differing properties. Polymers useful in the devices and methods of the present invention include, for example, stable or inert polymers, organic polymers, organic-inorganic copolymers, inorganic polymers, durable polymers, bioabsorbable, bioresorbable, resorbable, degradable, and biodegradable polymers. Those of skill in the art of polymer chemistry will be familiar with the different properties of polymeric compounds.

In some embodiments, the polymer comprises at least one of polyalkyl methacrylates, polyalkylene-co-vinyl acetates, polyalkylenes, polyurethanes, polyanhydrides, aliphatic polycarbonates, polyhydroxyalkanoates, silicone containing polymers, polyalkyl siloxanes, aliphatic polyesters, polyglycolides, polylactides, polylactide-co-glycolides, poly(e-caprolactone)s, polytetrahalooalkylenes, polystyrenes, poly(phosphasones), copolymers thereof, and combinations thereof.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)- and derivatives and copolymers such as those commonly sold as Teflon® products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Suitable polymers also include absorbable and/or resorbable polymers including the following, combinations, copolymers and derivatives of the following: Polylactides (PLA), Polyglycolides (PGA), PolyLactide-co-glycolides (PLGA), Polyanhydrides, Polyorthoesters, Poly(N-(2-hydroxypropyl)methacrylamide), Poly(1-aspartamide), including the derivatives DLPLA—poly(dl-lactide); LPLA—poly(1-lactide); PDO—poly(dioxanone); PGA-TMC poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); and PDO-PGA-TMC—poly(glycolide-co-trimethylene carbonate-co-dioxanone), and combinations thereof.

In some embodiments, the coating comprises a second polymer. The second polymer may comprise any polymer described herein. In some embodiments, the second polymer comprises PLGA having a weight ratio of 60:40 (1-lactide: glycolide). In some embodiments, the second polymer comprises PLGA having a weight ratio of 90:10 (1-lactide: glycolide). In some embodiments, the second polymer comprises PLGA having a weight ratio of between at least 90:10 (1-lactide: glycolide) and 60:40 (1-lactide: glycolide).

“Copolymer” as used herein refers to a polymer being composed of two or more different monomers. A copolymer may also and/or alternatively refer to random, block, graft, copolymers known to those of skill in the art.

“Biocompatible” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, causes inflammation or irritation, or induces an immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1 M NaOH at 37 degrees C. until complete degradation is observed. The solution is then neutralized with 1 M HCl. About 200 microliters of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The terms “bioabsorbable,” “biodegradable,” “bioerodible,” and “bioresorbable,” are art-recognized synonyms. These terms are used herein interchangeably. Bioabsorbable polymers typically differ from non-bioabsorbable polymers in that the former may be absorbed (e.g.; degraded) during use. In certain embodiments, such use involves in vivo use, such as in vivo therapy, and in other certain embodiments, such use involves in vitro use. In general, degradation attributable to biodegradability involves the degradation of a bioabsorbable polymer into its component subunits, or digestion, e.g., by a biochemical process, of the polymer into smaller, non-polymeric subunits. In certain embodiments, biodegradation may occur by enzymatic mediation, degradation in the presence of water (hydrolysis) and/or other chemical species in the body, or both. The bioabsorbabilty of a polymer may be shown in-vitro as described herein or by methods known to one of skill in the art. An in-vitro test for bioabsorbability of a polymer does not require living cells or other biologic materials to show bioabsorption properties (e.g. degradation, digestion). Thus, resorbtion, resorption, absorption, absorbtion, erosion, and dissolution may also be used synonymously with the terms “bioabsorbable,” “biodegradable,” “bioerodible,” and “bioresorbable.” Mechanisms of degradation of a bioaborbable polymer may include, but are not limited to, bulk degradation, surface erosion, and combinations thereof.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any bioabsorbable polymer is usually slower.

“Therapeutically desirable morphology” as used herein refers to the gross form and structure of the pharmaceutical agent, once deposited on the substrate, so as to provide for optimal conditions of ex vivo storage, in vivo preservation and/or in vivo release. Such optimal conditions may include, but are not limited to increased shelf life, increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the substrate prior to use or the location within the body of any biomedical implant. Preferably at least 10%, 20%, 30%, 40%, 50%, 60%, 70%, 80%, 90% or 100% of the pharmaceutical agent is in crystalline or semi-crystalline form.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipient that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Compressed fluid” as used herein refers to a fluid of appreciable density (e.g., >0.2 g/cc) that is a gas at standard temperature and pressure. “Supercritical fluid”, “near-critical fluid”, “near-supercritical fluid”, “critical fluid”, “densified fluid” or “densified gas” as used herein refers to a compressed fluid under conditions wherein the temperature is at least 80% of the critical temperature of the fluid and the pressure is at least 50% of the critical pressure of the fluid, a density of +50% of the critical density of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydrofluorocarbons, perfluorocarbons (such as perfluoromethane and perfluoropropane, chloroform, trichloro-fluoromethane, dichloro-difluoromethane, dichloro-tetrafluoroethane) and mixtures thereof. In some embodiments, the supercritical fluid is hexafluoropropane (FC-236EA), or 1,1,1,2,3,3-hexafluoropropane. In some embodiments, the supercritical fluid is hexafluoropropane (FC-236EA), or 1,1,1,2,3,3-hexafluoropropane for use in PLGA polymer coatings.

“Sintering” as used herein refers to the process by which parts of the matrix or the entire polymer matrix becomes continuous (e.g., formation of a continuous polymer film). As discussed below, the sintering process is controlled to produce a fully conformal continuous matrix (complete sintering) or to produce regions or domains of continuous coating while producing voids (discontinuities) in the matrix. As well, the sintering process is controlled such that some phase separation is obtained between polymer different polymers (e.g., polymers A and B) and/or to produce phase separation between discrete polymer particles. Through the sintering process, the adhesions properties of the coating are improved to reduce flaking of detachment of the coating from the substrate during manipulation in use. As described below, in some embodiments, the sintering process is controlled to provide incomplete sintering of the polymer matrix. In embodiments involving incomplete sintering, a polymer matrix is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to treat a substrate that has been coated with a polymer and a drug, using dry powder and RESS electrostatic coating processes. In another example, isobutylene is employed in the sintering process. In other examples a mixture of carbon dioxide and isobutylene is employed. In another example, 1,1,2,3,3-hexafluoropropane is employed in the sintering process.

When an amorphous material is heated to a temperature above its glass transition temperature, or when a crystalline material is heated to a temperature above a phase transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes autoxidation of drug, whether in amorphous, semi-crystalline, or crystalline form, by reducing exposure thereof to free radicals, residual solvents and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide or other appropriate gas is employed to prevent electrical charge is transferred from the substrate to the surrounding environment.

“Bulk properties” properties of a coating including a pharmaceutical or a biological agent that can be enhanced through the methods of the invention include for example: adhesion, smoothness, conformality, thickness, and compositional mixing.

“Electrostatically charged” or “electrical potential” or “electrostatic capture” or “e-” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the fluid medium of the capture vessel onto the surface of the substrate is enhanced via electrostatic attraction. This may be achieved by charging the particles and grounding the substrate or conversely charging the substrate and grounding the particles, or by some other process, which would be easily envisaged by one of skill in the art of electrostatic capture.

“Intimate mixture” as used herein, refers to two or more materials, compounds, or substances that are uniformly distributed or dispersed together.

“Layer” as used herein refers to a material covering a surface or forming an overlying part or segment. Two different layers may have overlapping portions whereby material from one layer may be in contact with material from another layer. Contact between materials of different layers can be measured by determining a distance between the materials. For example, Raman spectroscopy may be employed in identifying materials from two layers present in close proximity to each other.

While layers defined by uniform thickness and/or regular shape are contemplated herein, several embodiments described below relate to layers having varying thickness and/or irregular shape. Material of one layer may extend into the space largely occupied by material of another layer. For example, in a coating having three layers formed in sequence as a first polymer layer, a pharmaceutical agent layer and a second polymer layer, material from the second polymer layer which is deposited last in this sequence may extend into the space largely occupied by material of the pharmaceutical agent layer whereby material from the second polymer layer may have contact with material from the pharmaceutical layer. It is also contemplated that material from the second polymer layer may extend through the entire layer largely occupied by pharmaceutical agent and contact material from the first polymer layer.

It should be noted however that contact between material from the second polymer layer (or the first polymer layer) and material from the pharmaceutical agent layer (e.g.; a pharmaceutical agent crystal particle or a portion thereof) does not necessarily imply formation of a mixture between the material from the first or second polymer layers and material from the pharmaceutical agent layer. In some embodiments, a layer may be defined by the physical three-dimensional space occupied by crystalline particles of a pharmaceutical agent (and/or biological agent). It is contemplated that such layer may or may not be continuous as phhysical space occupied by the crystal particles of pharmaceutical agents may be interrupted, for example, by polymer material from an adjacent polymer layer. An adjacent polymer layer may be a layer that is in physical proximity to be pharmaceutical agent particles in the pharmaceutical agent layer. Similarly, an adjacent layer may be the layer formed in a process step right before or right after the process step in which pharmaceutical agent particles are deposited to form the pharmaceutical agent layer.

As described below, material deposition and layer formation provided herein are advantageous in that the pharmaceutical agent remains largely in crystalline form during the entire process. While the polymer particles and the pharmaceutical agent particles may be in contact, the layer formation process is controlled to avoid formation of a mixture between the pharmaceutical agent particles the polymer particles during formation of a coated device.

“Laminate coating” as used herein refers to a coating made up of two or more layers of material. Means for creating a laminate coating as described herein (e.g.; a laminate coating comprising bioabsorbable polymer(s) and pharmaceutical agent) may include coating the stent with drug and polymer as described herein (e-RESS, e-DPC, compressed-gas sintering). The process comprises performing multiple and sequential coating steps (with sintering steps for polymer materials) wherein different materials may be deposited in each step, thus creating a laminated structure with a multitude of layers (at least 2 layers) including polymer layers and pharmaceutical agent layers to build the final device (e.g.; laminate coated stent).

The coating methods provided herein may be calibrated to provide a coating bias whereby the mount of polymer and pharmaceutical agent deposited in the abluminal surface of the stent (exterior surface of the stent) is greater than the amount of pharmaceutical agent and amount of polymer deposited on the luminal surface of the stent (interior surface of the stent). The resulting configuration may be desirable to provide preferential elution of the drug toward the vessel wall (luminal surface of the stent) where the therapeutic effect of anti-restenosis is desired, without providing the same antiproliferative drug(s) on the abluminal surface, where they may retard healing, which in turn is suspected to be a cause of late-stage safety problems with current DESs.

As well, the methods described herein provide a device wherein the coating on the stent is biased in favor of increased coating at the ends of the stent. For example, a stent having three portions along the length of the stent (e.g.; a central portion flanked by two end portions) may have end portions coated with increased amounts of pharmaceutical agent and/or polymer compared to the central portion.

Means for creating the bioabsorbable polymer(s)+drug (s) matrix on the stent-form—forming the final device:
Spray coat the stent-form with drug and polymer as is done in Micell process (e-RESS, e-DPC, compressed-gas sintering).
Perform multiple and sequential coating—sintering steps where different materials may be deposited in each step, thus creating a laminated structure with a multitude of thin layers of drug(s), polymer(s) or drug+polymer that build the final stent.
Perform the deposition of polymer(s)+drug(s) laminates with the inclusion of a mask on the inner (luminal) surface of the stent. Such a mask could be as simple as a non-conductive mandrel inserted through the internal diameter of the stent form. This masking could take place prior to any layers being added, or be purposefully inserted after several layers are deposited continuously around the entire stent-form.

Another advantage of the present invention is the ability to create a stent with a controlled (dialed-in) drug-elution profile. Via the ability to have different materials in each layer of the laminate structure and the ability to control the location of drug(s) independently in these layers, the method enables a stent that could release drugs at very specific elution profiles, programmed sequential and/or parallel elution profiles. Also, the present invention allows controlled elution of one drug without affecting the elution of a second drug (or different doses of the same drug).

The embodiments incorporating a stent form or framework provide the ability to radiographically monitor the stent in deployment. In an alternative embodiment, the inner-diameter of the stent can be masked (e.g. by a non-conductive mandrel). Such masking would prevent additional layers from being on the interior diameter (abluminal) surface of the stent. The resulting configuration may be desirable to provide preferential elution of the drug toward the vessel wall (luminal surface of the stent) where the therapeutic effect of anti-restenosis is desired, without providing the same antiproliferative drug(s) on the abluminal surface, where they may retard healing, which in turn is suspected to be a cause of late-stage safety problems with current DESs.

The present invention provides numerous advantages. The invention is advantageous allows for employing a platform combining layer formation methods based on compressed fluid technologies; electrostatic capture and sintering methods. The platform results in drug eluting stents having enhanced therapeutic and mechanical properties. The invention is particularly advantageous in that it employs optimized laminate polymer technology. In particular, the present invention allows the formation of discrete layers of specific drug platforms.

Conventional processes for spray coating stents require that drug and polymer be dissolved in solvent or mutual solvent before spray coating can occur. The platform provided herein the drugs and polymers are coated on the stent in discrete steps, which can be carried out simultaneously or alternately. This allows discrete deposition of the active agent (e.g.; a drug) within a polymer matrix thereby allowing the placement of more than one drug on a single medical device with or without an intervening polymer layer. For example, the present platform provides a dual drug eluting stent.

Some of the advantages provided by the subject invention include employing compressed fluids (e.g., supercritical fluids, for example E-RESS based methods); solvent free deposition methodology; a platform that allows processing at lower temperatures thereby preserving the qualities of the active agent and the polymer matrix; the ability to incorporate two, three or more drugs while minimizing deleterious effects from direct interactions between the various drugs and/or their excipients during the fabrication and/or storage of the drug eluting stents; a dry deposition; enhanced adhesion and mechanical properties of the layers on the stent; precision deposition and rapid batch processing; and ability to form intricate structures.

In one embodiment, the present invention provides a multi-drug delivery platform which produces strong, resilient and flexible drug eluting stents including an anti-restenosis drug (e.g.; a limus or taxol) and anti-thrombosis drug (e.g.; heparin or an analog thereof) and well characterized bioabsorbable polymers. The drug eluting stents provided herein minimize potential for thrombosis, in part, by reducing or totally eliminating thrombogenic polymers and reducing or totally eliminating residual drugs that could inhibit healing.

The platform provides optimized delivery of multiple drug therapies for example for early stage treatment (restenosis) and late-stage (thrombosis).

The platform also provides an adherent coating which enables access through tortuous lesions without the risk of the coating being compromised.

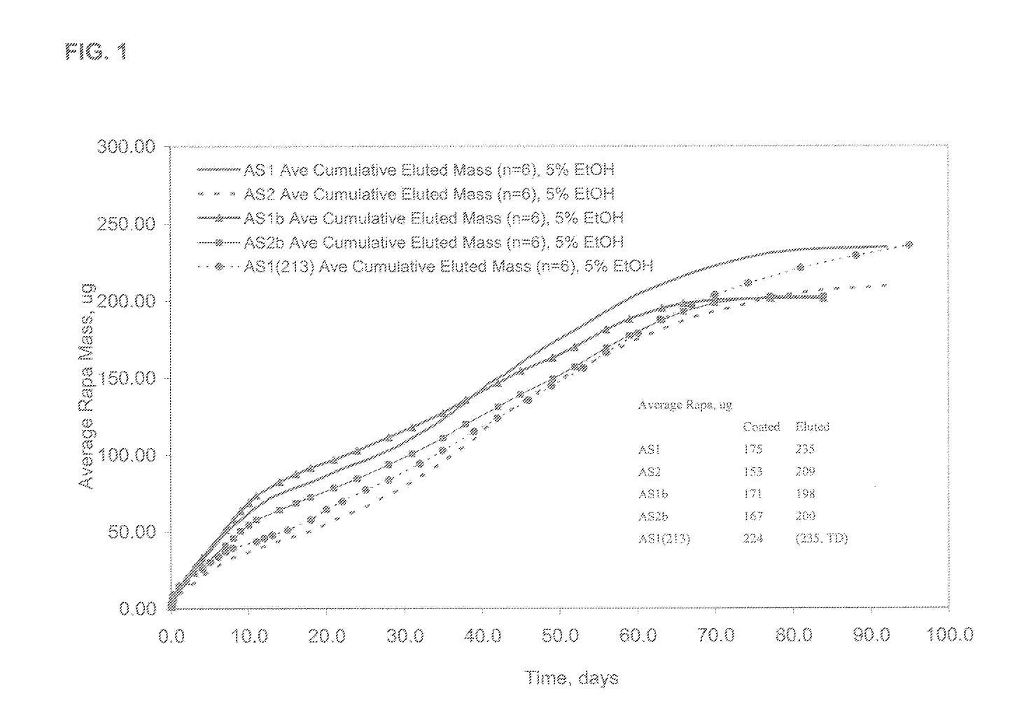

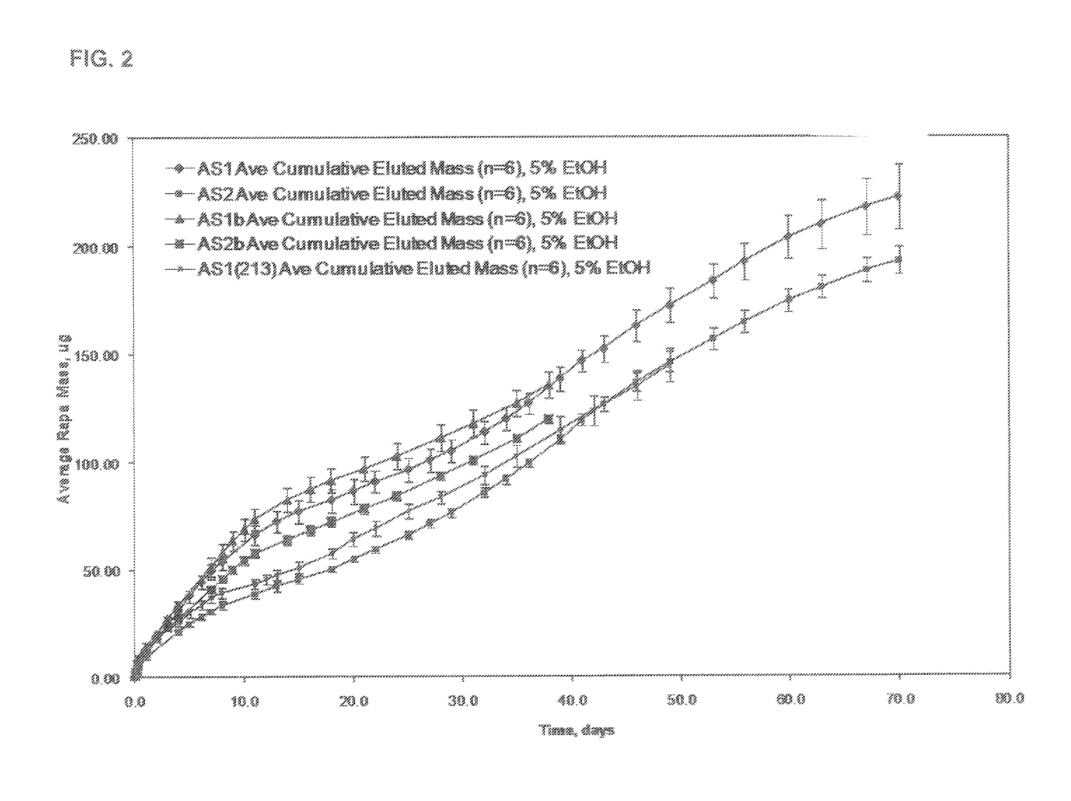

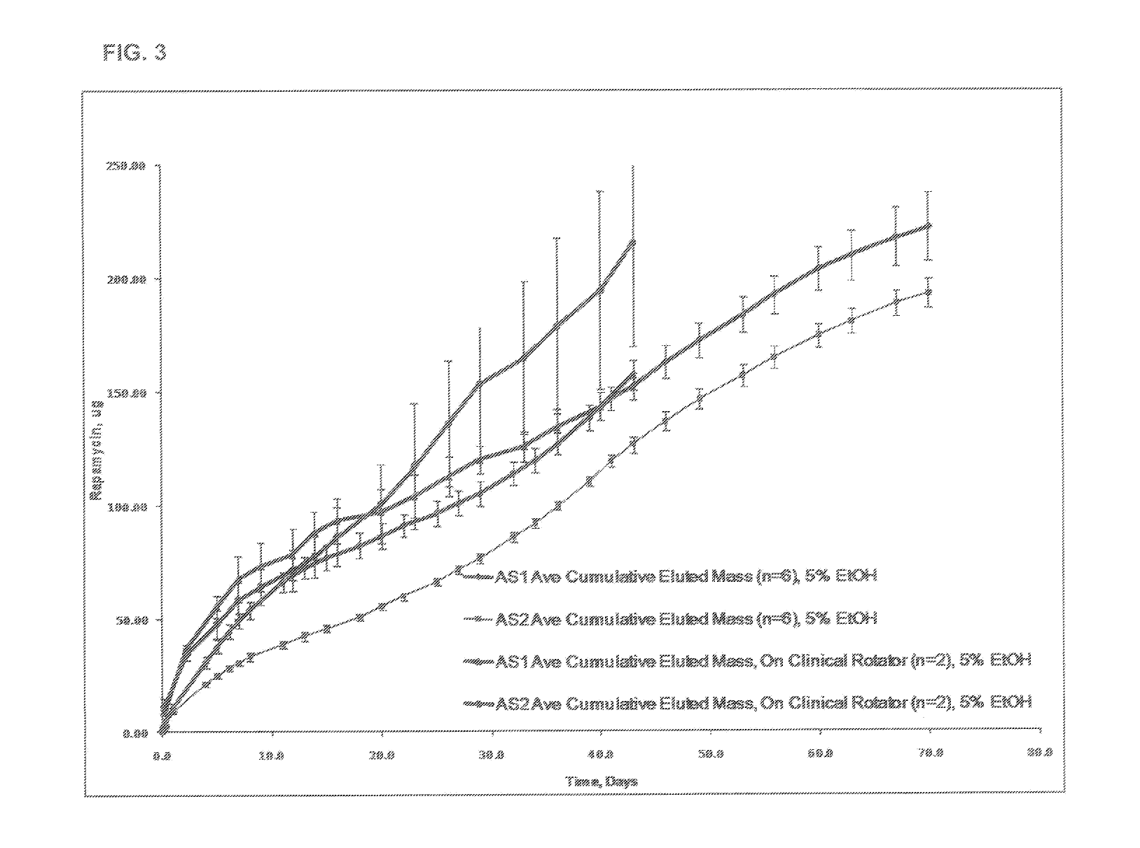

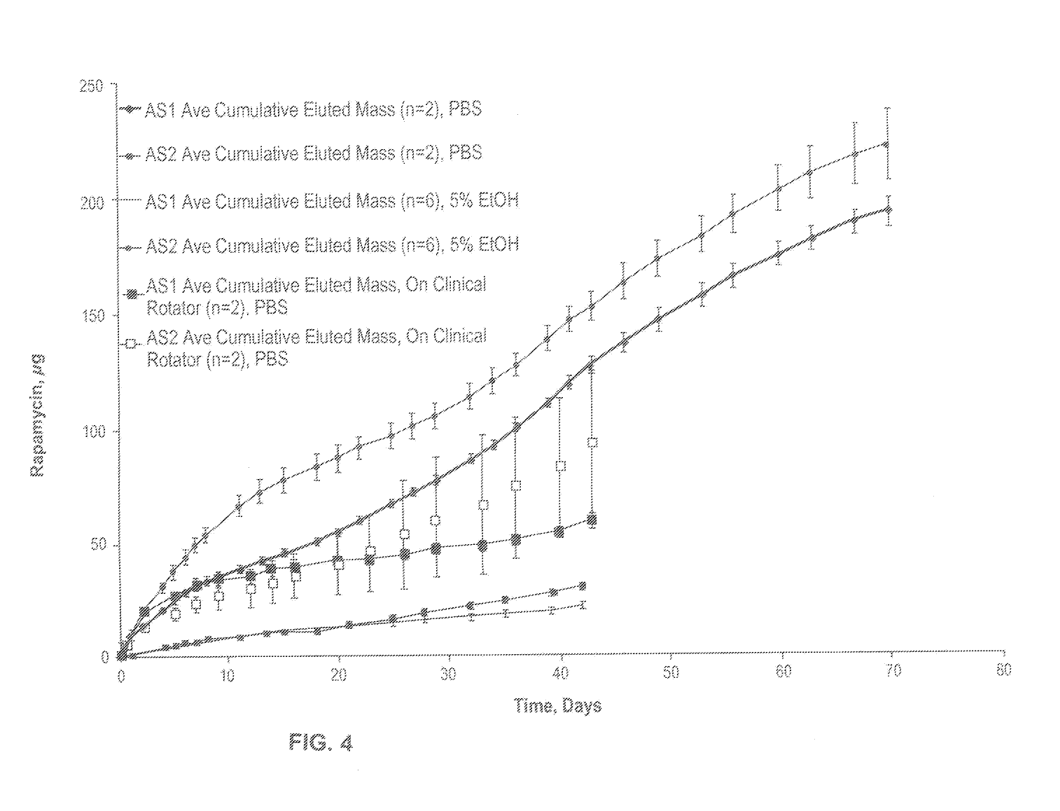

Another advantage of the present platform is the ability to provide highly desirable eluting profiles (e.g., the profile illustrated in FIGS. 1-4).

Advantages of the invention include the ability to reduce or completely eliminate potentially thrombogenic polymers as well as possibly residual drugs that may inhibit long term healing. As well, the invention provides advantageous stents having optimized strength and resilience if coatings which in turn allows access to complex lesions and reduces or completely eliminates delamination. Laminated layers of bioabsorbable polymers allow controlled elution of one or more drugs.

The platform provided herein reduces or completely eliminates shortcoming that have been associated with conventional drug eluting stents. For example, the platform provided herein allows for much better tuning of the period of time for the active agent to elute and the period of time necessary for the polymer matrix to resorb thereby minimizing thrombosis and other deleterious effects associate with poorly controlled drug release.

The present invention provides several advantages which overcome or attenuate the limitations of current technology for bioabsorbable stents. For example, an inherent limitation of conventional bioabsorbable polymeric materials relates to the difficulty in forming to a strong, flexible, deformable (e.g. balloon deployable) stent with low profile. The polymers generally lack the strength of high-performance metals. The present invention overcomes these limitations by creating a laminate structure in the essentially polymeric stent. Without wishing to be bound by any specific theory or analogy, the increased strength provided by the stents of the invention can be understood by comparing the strength of plywood vs. the strength of a thin sheet of wood.

Embodiments of the invention involving a thin metallic stent-framework provide advantages including the ability to overcome the inherent elasticity of most polymers. It is generally difficult to obtain a high rate (e.g., 100%) of plastic deformation in polymers (compared to elastic deformation where the materials have some ‘spring back’ to the original shape). Again, without wishing to be bound by any theory, the central metal stent (that would be too small and weak to serve as a stent itself) would act like wires inside of a plastic, deformable stent, basically overcoming any ‘elastic memory’ of the polymer.

Provided herein is a coated stent having a plurality of stent struts for delivery to a body lumen comprising a stent and a coating comprising a pharmaceutical agent and a polymer wherein at least part of the drug is in crystalline form and wherein the coating is substantially resistant to stent strut breakage. The body lumen may include a peripheral body lumen, and/or a coronary body lumen.

A coating may be susbtantially resistant to strut breakage if the coating is not completely penetrated by the strut following strut fracture. The fracture need not be a complete stent strut break, although it may be. Thus, in some embodiments, the coating may be any precent less than 100% penetrated and still be substantially resistant to strut breakage. In some embodiments, the coating is substantially resistant to strut breakage wherein the coating is at most 10% penetrated following a stent strut breakage. In some embodiments, the coating is substantially resistant to strut breakage wherein the coating is at most 20% penetrated following a stent strut breakage. In some embodiments, the coating is substantially resistant to strut breakage wherein the coating is at most 25% penetrated following a stent strut breakage. In some embodiments, the coating is substantially resistant to strut breakage wherein the coating is at most 30% penetrated following a stent strut breakage. In some embodiments, the coating is substantially resistant to strut breakage wherein the coating is at most 40% penetrated following a stent strut breakage. In some embodiments, the coating is substantially resistant to strut breakage wherein the coating is at most 50% penetrated following a stent strut breakage. In some embodiments, the coating is substantially resistant to strut breakage wherein the coating is at most 60% penetrated following a stent strut breakage. In some embodiments, the coating is substantially resistant to strut breakage wherein the coating is at most 70% penetrated following a stent strut breakage. In some embodiments, the coating is substantially resistant to strut breakage wherein the coating is at most 75% penetrated following a stent strut breakage. In some embodiments, the coating is substantially resistant to strut breakage wherein the coating is at most 80% penetrated following a stent strut breakage. In some embodiments, the coating is substantially resistant to strut breakage wherein the coating is at most 90% penetrated following a stent strut breakage. In some embodiments, the coating is substantially resistant to strut breakage wherein the coating is at most 95% penetrated following a stent strut breakage. In some embodiments, the coating is substantially resistant to strut breakage wherein the coating is less than 100% penetrated following a stent strut breakage.

In some embodiments, the polymer comprises a durable polymer. The polymer may include a cross-linked durable polymer. Example biocomaptible durable polymers include, but are not limited to, polystyrenes acrylates, epoxies. The polymer may include a thermoset material. In some embodiments, the durable polymer comprises at least one of a polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, celluiosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof. The polymer may provide radial strength for the coated stent. The polymer may provide durability for the coated stent. The polymer may shield the body lumen from contact with a broken strut of the stent. The polymer may be impenetrable by a broken strut of the stent. The stent may be thin to be a base for the polymer to build upon, and the polymer itself may provide the radial strength and durability to withstand the forces encountered in the body, including but not limited to internal forces from blood flow, and external forces, such as may be encountered in peripheral vessels and other body lumens. The coated stents provided herein may be peripheral stents which may be delivered to vessels not protected by the rib cage and which may need to flex or withstand external forces without plastic deformation of the stent and without breaking struts of the stent. The coatings and coating methods provided herein provide substantial protection from these by establishing a multi-layer coating which can be bioabsorbable or durable or a combination thereof, and which can both deliver drugs and provide elasticity and radial strength for the vessel in which it is delivered.

In some embodiments, the polymer comprises a bioabsorbable polymer. In some embodiments, the polymer comprises a cross-linked bioabsorbable polymer.

In some embodimetns, the coating comprises a plurality of layers deposited on said stent to form said coated stent. The coating may comprise five layers deposited as follows: a first polymer layer, a first drug layer, a second polymer layer, a second drug layer and a third polymer layer. In some embodiments, the drug and polymer are in the same layer; in separate layers or form overlapping layers. In some embodiments, plurality of layers comprises at least 4 or more layers. In some embodiments, the plurality of layers comprises 10, 20, 50, or 100 layers. In some embodiments, the plurality of layers comprises at least one of: at least 10, at least 20, at least 50, and at least 100 layers. In some embodiments, the plurality of layers comprises alternate drug and polymer layers. The drug layers may be substantially free of polymer and/or the polymer layers may be substantially free of drug.

In some embodiments the coating comprises a fiber reinforcement. The fiber reinforcement may comprise a natural or a synthetic fiber. Examples of the fiber reinforcement may include any biocompatible fiber known in the art. This may, for non-limiting example, include any reinforcing fiber from silk to catgut to polymers (as described elsewhere herein) to olefins to acrylates. The fiber may be deposited according to methods disclosed herein, including by RESS. The concentration for a reinforcing fiber that is or comprises a polymer may be any concentration of the fiber forming polymer from 5 to 50 miligrams per milliliter and deposited according to the RESS process. For exmaple, methods of depositing the fiber may comprise and/or adapt methods described in Levit, et al., “Supercritical CO2 Assisted Electrospinning” J. of Supercritical Fluids, 329-333, Vol 31, Issue 3, (November 2004). In some embodiments, the fiber reinforcement is deposited on the stent in dry form. In some embodiments, depositing the fiber reinforcement on the stent meants to deposit the fiber reinforcement on another element of the coating (i.e. the pharmaceutical agent, the polymer, and/or another coating element). The fiber reinforcement need not be deposited directly on the stent in order to be deposited on the stent as part of the coating. The fiber reinforcement may be a part of another coating layer, such as a polymer layer or an active agent layer. The fiber may comprise a length to diameter ratio of at least 3:1, in some embodiments. The fiber may comprise lengths of at least 200 nanometers. The fiber may comprise lengths of up to 5 micrometers in certain embodiments. The fiber may comprise lengths of 200 nanometers to 5 micrometers, in some embodiments.

Provided herein is a coated stent having a plurality of stent struts for delivery to a body lumen comprising a stent and a coating comprising a pharmaceutical agent and a polymer wherein at least part of the drug is in crystalline form and wherein the coating provides a release profile whereby the pharmaceutical agent is released over a period longer than two weeks. The body lumen may include a peripheral body lumen, and/or a coronary body lumen. A peripheral vessel may have a large lesion site, and is, generally speaking, longer than a coronary vessesl lesion (although it may not be). The drug amount necessary to treat such a vessel may be required to elute over a longer time than for a coronary lesion, or another small lesion. The coatings and methods provided herein can be formulated to provide longer elution because of the way the layers of drug and polymer are constructed and formed, as described herein.

Provided herein are devices and methods adapted for the peripheral vessels of the vasculature, which may exhibit symptoms of peripheral artery disease. These vessels may require release of a drug which extends over a longer period of time than a coronary lesion might, thus, the methods and devices provided herein can be formulated to provide extended release of the drug by controlling the release such that a minimal of drug is washed away over time allowing more of the actual drug deposited on the substrate to be eluted into the vessel. This provides a higher ratio of therapeutic drug to drug lost during delivery and post delivery, and thus the total amount of drug can be lower if less is lost during and post delivery. This can be useful for drugs which may have higher toxicities at lower concentrations, but which may be therapeutic nonetheless if properly controlled. The methods and devices provided herein are capable of eluting the drug in a more controlled manner, and, thus, less drug overall is deposited on the substrate when less is lost by being washed away during and post delivery to the delivery site.

In some embodiments, the coating provides a release profile whereby the drug is released over a period longer than 1 month. In some embodiments, the coating provides a release profile whereby the drug is released over a period longer than 2 months. In some embodiments, the coating provides a release profile whereby the drug is released over a period longer than 3 months. In some embodiments, the coating provides a release profile whereby the drug is released over a period longer than 4 months. In some embodiments, the coating provides a release profile whereby the drug is released over a period longer than 6 months. In some embodiments, the coating provides a release profile whereby the pharmaceutical agent is released over a period longer than twelve months.

In some embodiments, over 1% of said pharmaceutical agent coated on said stent is delivered to the vessel. In some embodiments, over 2% of said pharmaceutical agent coated on said stent is delivered to the vessel. In some embodiments, over 5% of said pharmaceutical agent coated on said stent is delivered to the vessel. In some embodiments, over 10% of said pharmaceutical agent coated on said stent is delivered to the vessel. In some embodiments, over 25% of said pharmaceutical agent coated on said stent is delivered to the vessel. In some embodiments, over 50% of said pharmaceutical agent coated on said stent is delivered to the vessel.

In some embodiments, the agent and polymer coating has substantially uniform thickness and drug in the coating is substantially uniformly dispersed within the agent and polymer coating.

In some embodiments, the coated stent provides an elution profile wherein about 10% to about 50% of drug is eluted at week 20 after the stent is implanted in a subject under physiological conditions, about 25% to about 75% of drug is eluted at week 30 and about 50% to about 100% of drug is eluted at week 50.

In some embodiments, the pharmaceutical agent is detected in vivo after two weeks by blood concentration testing as noted elsewhere herein. In some embodiments, the pharmaceutical agent is detected in-vitro after a two weeks time period or a correlatable time period thereof by elution testing in 37 degree buffered saline at infinite sink conditions and/or according to elution testing methods noted elsewhere herein.

Some embodiments of the coating further comprises an anti-inflammatory agent.

In some embodiments, the macrolide-polymer coating comprises one or more resorbable polymers. In some embodiments, one or more resorbable polymers are selected from PLGA (poly(lactide-co-glycolide); DLPLA—poly(dl-lactide); LPLA—poly(1-lactide); PGA polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

In some embodiments, the polymer is 50/50 PLGA.

Provided herein is a coated stent having a plurality of stent struts for delivery to a body lumen comprising a stent and a coating comprising a pharmaceutical agent and a polymer wherein at least part of the drug is in crystalline form and wherein said coating is substantially conformal to the stent struts when the coated stent is in an expanded state. The body lumen may include a peripheral body lumen, and/or a coronary body lumen.

Peripheral stent delivery sites are, typically (although not always), larger in diameter as compared to a coronary stent delivery site. Thus the stents delivered to that location need to be larger in diameter. Nevertheless, as a minimally invasive techinique, the peripheral stent also needs to be collapsed (and/or crimped) to a small diameter for delivery to the site, then expanded to a final diameter. Coating a stent having highter ratios of collapsed state to expanded state as compared to a coronary stent presents new challenges since the coating must withstand the expansion ratio without substantial cracking, tearing, and creation of other coating defects that might alter the elution of the drug from the coating into the vessel. The coatings (on the coated stents) and methods provided herein can alleviate these defects by providing a way to coat the stents that is substantially conformal to the stent even in the expanded state. In some embodiments, the coated stent in the expanded state is at least about 99.99% free of coating defects. In some embodiments, the coated stent in the expanded state is at least about 99.99% free of coating defects. In some embodiments, the coated stent in the expanded state is at least about 99.9% free of coating defects. In some embodiments, the coated stent in the expanded state is at least about 99.0% free of coating defects. In some embodiments, the coated stent in the expanded state is at least about 98% free of coating defects. In some embodiments, the coated stent in the expanded state is at least about 97% free of coating defects. In some embodiments, the coated stent in the expanded state is at least about 95% free of coating defects. In some embodiments, the coated stent in the expanded state is at least about 94% free of coating defects. In some embodiments, the coated stent in the expanded state is at least about 93% free of coating defects. In some embodiments, the coated stent in the expanded state is at least about 92% free of coating defects. In some embodiments, the coated stent in the expanded state is at least about 90% free of coating defects. In some embodiments, the coated stent in the expanded state is at least about 85% free of coating defects. In some embodiments, the coated stent in the expanded state is at least about 80% free of coating defects. In some embodiments, the coated stent in the expanded state is at least about 75% free of coating defects. “About” when referring to coating defects, means plus or minus 0.01%-0.10%, plus or minus 0.1%-0.5%, plus or minus 0.5% to 1%, or plus or minus 1% to 5%. Coating defects may include at least one of cracks, bubbles, bare spots, bald spots, flaps, lifted coating, webs, and other visual defects.

In some embodiments, the coating is applied when the stent is in a collapsed state. In some embodiments, the coated stent has a radial expansion ratio of about 1 in a collapsed state up to about 3.0 in the expanded state. In some embodiments, the coated stent has a radial expansion ratio of about 1 in a collapsed state up to about 4.0 in the expanded state. In some embodiments, the coated stent has a radial expansion ratio of about 1 in a collapsed state up to about 5.0 in the expanded state. In some embodiments, the coated stent has a radial expansion ratio of about 1 in a collapsed state up to about 6.0 in the expanded state. In some embodiments, the coated stent has a radial expansion ratio of about 1 in a collapsed state to over about 3.0 in the expanded state. In some embodiments, the coated stent has a radial expansion ratio of about 1 in a collapsed state to over about 4.0 in the expanded state.

In some embodiments, the pharmaceutical agent comprises one or more of an antirestenotic agent, antidiabetic, analgesic, antiinflammatory agent, antirheumatic, antihypotensive agent, antihypertensive agent, psychoactive drug, tranquillizer, antiemetic, muscle relaxant, glucocorticoid, agent for treating ulcerative colitis or Crohn's disease, antiallergic, antibiotic, antiepileptic, anticoagulant, antimycotic, antitussive, arteriosclerosis remedy, diuretic, protein, peptide, enzyme, enzyme inhibitor, gout remedy, hormone and inhibitor thereof, cardiac glycoside, immunotherapeutic agent and cytokine, laxative, lipid-lowering agent, migraine remedie, mineral product, otological, anti parkinson agent, thyroid therapeutic agent, spasmolytic, platelet aggregation inhibitor, vitamin, cytostatic and metastasis inhibitor, phytopharmaceutical, chemotherapeutic agent and amino acid, acarbose, antigen, beta-receptor blocker, non-steroidal antiinflammatory drug {NSAIDs], cardiac glycosides acetylsalicylic acid, virustatic, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, and zotipine.

In some embodiments, the pharmaceutical agent comprises a macrolide immunosuppressive (limus) drug. The macrolide immunosuppressive drug may comprise one or more of rapamycin, biolimus (biolimus A9), 4O—O-(2-Hydroxyethyl)rapamycin (everolimus), 40-O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin 4O—O-(2-Hydroxy)ethoxycar-bonylmethyl-rapamycin, 4O—O-(3-Hydroxy)propyl-rapamycin 4O—O-(6-Hydroxy)hexyl-rapamycin 4O—O-[2-(2-Hydroxy)ethoxy]ethyl-rapamycin 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the coating further comprises an anti-inflammatory agent.

In some embodiments, at least part of said drug forms a phase separate from one or more phases formed by said polymer.

In some embodiments, the drug is at least 50% crystalline. In some embodiments, the drug is at least 75% crystalline. In some embodiments, the drug is at least 90% crystalline. In some embodiments, the drug is at least 95% crystalline. In some embodiments, the drug is at least 99% crystalline.

In some embodiments, the polymer is a mixture of two or more polymers. In some embodiments, the mixture of polymers forms a continuous film around particles of drug. The two or more polymers may be intimately mixed. The mixture may comprise no single polymer domain larger than about 20 nm. Each polymer in said mixture may comprise a discrete phase. Discrete phases formed by said polymers in said mixture may be larger than about 10 nm. Discrete phases formed by said polymers in said mixture may be larger than about 50 nm.

In some embodiments, the stent comprises at least one of stainless steel, a cobalt-chromium alloy, tantalum, platinum, Nitinol™, gold, a NiTi alloy, and a thermoplastic polymer.

In some embodiments, the stent is formed from a metal alloy.

In some embodiments, the stent is capable of retaining its expanded condition upon the expansion thereof.

In some embodiments, the stent is formed from a material that plastically deforms when subjected to at least 4 atmospheres of pressure. In some embodiments, the stent is formed from a material that plastically deforms when subjected to at least 2 atmospheres of pressure. In some embodiments, the stent is formed from a material that plastically deforms when subjected to at least 5 atmospheres of pressure. In some embodiments, the stent is formed from a material that plastically deforms when subjected to at least 6 atmospheres of pressure.

In some embodiments, the stent is formed from a material that is capable of self-expansion in the body lumen.

In some embodiments, the stent is formed from a super-elastic metal alloy which transforms from an austenitic state to a martensitic state in the body lumen. In some embodiments, the stent is formed from a super-elastic metal alloy that is capable of deformation from a martensitic state to an austenitic state when the stent is mounted on a catheter. In some embodiments, the stent exhibits linear pseudoelasticity when stressed. In some embodiments, the stent is formed from a super-elastic metal alloy having a transformation temperature greater than a mammalian body temperature.

In some embodiments, at least one of the stent and the polymer is formed of a radiopaque material. In some embodiments, the stent comprises at least one of: iridium, platinum, gold, rhenium, tungsten, palladium, rhodium, tantalum, silver, ruthenium, chromium, iron, cobalt, vanadium, manganese, boron, copper, aluminum, niobium, zirconium, and hafnium.

In some embodiments, heparin is attached to the stent by reaction with an aminated silane. In some embodiments, the stent is coated with a silane monolayer.

In some embodiments, onset of heparin anti-coagulant activity is obtained at week 3 or later. In some embodiments, heparin anti-coagulant activity remains at an effective level at least 90 days after onset of heparin activity. In some embodiments, heparin anti-coagulant activity remains at an effective level at least 120 days after onset of heparin activity. In some embodiments, the heparin anti-coagulant activity remains at an effective level at least 200 days after onset of heparin activity.

In some embodiments, the stent is adapted for delivery to at least one of a peripheral artery, a peripheral vein, a carotid artery, a vein, an aorta, and a biliary duct. In some embodiments, the stent is adapted for delivery to a superficial femoral artery. The stent may be adapted for delivery to a tibial artery. The stent may be adapted for delivery to a renal artery. The stent may be adapted for delivery to an iliac artery. The stent may be adapted for delivery to a bifurcated vessel. The stent is adapted for delivery to a vessel having a side branch at an intended delivery site of the vessel. The stent is adapted for delivery to the side branch of the vessel.

Provided herein is a method for preparing a coated stent for delivery to a body lumen comprising the following steps: providing a stent, forming a coating comprising a pharmaceutical agent and a polymer on the stent wherein at least part of the drug is in crystalline form, and wherein the coating is substantially resistant to stent strut breakage. The body lumen may include a peripheral body lumen, and/or a coronary body lumen.

Provided herein is a method for preparing a coated stent for delivery to a body lumen comprising the following steps: providing a stent; forming a coating comprising a pharmaceutical agent and a polymer coating on the stent wherein at least part of the drug is in crystalline form, and wherein the coating provides a release profile whereby the pharmaceutical agent is released over a period longer than 2 weeks. The body lumen may include a peripheral body lumen, and/or a coronary body lumen.

Provided herein is a method for preparing a coated stent for delivery to a body lumen comprising the following steps: providing a stent; forming a coating comprising a pharmaceutical agent and a polymer on the stent wherein at least part of the drug is in crystalline form, and wherein said coating is substantially conformal to the stent struts when the coated stent is in an expanded state. The body lumen may include a peripheral body lumen, and/or a coronary body lumen.

In some embodiments, forming the coating comprises depositing the drug in dry powder form.

In some embodiments, forming the coating comprises depositing the polymer in dry powder form.

In some embodiments, forming the coating comprises depositing the polymer by an e-SEDS process.

In some embodiments, forming the coating comprises depositing the polymer by an e-RESS process.

In some embodiments, the method comprises comprises sintering said coating under conditions that do not substantially modify the morphology of said drug.

In some embodiments, the pharmaceutical agent comprises one or more of an antirestenotic agent, antidiabetic, analgesic, antiinflammatory agent, antirheumatic, antihypotensive agent, antihypertensive agent, psychoactive drug, tranquillizer, antiemetic, muscle relaxant, glucocorticoid, agent for treating ulcerative colitis or Crohn's disease, antiallergic, antibiotic, antiepileptic, anticoagulant, antimycotic, antitussive, arteriosclerosis remedy, diuretic, protein, peptide, enzyme, enzyme inhibitor, gout remedy, hormone and inhibitor thereof, cardiac glycoside, immunotherapeutic agent and cytokine, laxative, lipid-lowering agent, migraine remedie, mineral product, otological, anti parkinson agent, thyroid therapeutic agent, spasmolytic, platelet aggregation inhibitor, vitamin, cytostatic and metastasis inhibitor, phytopharmaceutical, chemotherapeutic agent and amino acid, acarbose, antigen, beta-receptor blocker, non-steroidal antiinflammatory drug {NSAIDs], cardiac glycosides acetylsalicylic acid, virustatic, aclarubicin, acyclovir, cisplatin, actinomycin, alpha- and beta-sympatomimetics, (dmeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, and zotipine.

In some embodiments, the pharmaceutical agent comprises a macrolide immunosuppressive drug, and the macrolide immunosuppressive drug comprises one or more of rapamycin, biolimus (biolimus A9), 4O—O-(2-Hydroxyethyl)rapamycin (everolimus), 4O—O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin 4O—O-(2-Hydroxy)ethoxycar-bonylmethyl-rapamycin, 4O—O-(3-Hydroxy)propyl-rapamycin 4O—O-(6-Hydroxy)hexyl-rapamycin 4O—O-[2-(2-Hydroxy)ethoxy]ethyl-rapamycin 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), and 42-[3-hydroxy-2-(hydroxymethyl)-2-methylpropanoate]rapamycin (temsirolimus) (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the polymer comprises a bioabsorbable polymer and wherein forming the coating comprises depositing the bioabsorbable polymer in dry powder form.

In some embodiments, one or more bioabsorbable polymers are selected from PLGA (poly(lactide-co-glycolide); DLPLA—poly(dl-lactide); LPLA—poly(1-lactide); PGA polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone).

In some embodiments, the coating comprises a second polymer. The second polymer may comprise any polymer described herein. In some embodiments, the second polymer comprises PLGA having a weight ratio of 60:40 (1-lactide: glycolide). In some embodiments, the second polymer comprises PLGA having a weight ratio of 90:10 (1-lactide: glycolide). In some embodiments, the second polymer comprises PLGA having a weight ratio of between at least 90:10 (1-lactide: glycolide) and 60:40 (1-lactide: glycolide).

In some embodiments, the bioabsorbable polymer is cross-linked. In some embodiments, the polymer comprises a durable polymer, and wherein forming the coating comprises depositing the durable polymer in dry powder form. In some embodiments, the durable polymer is cross-linked. In some embodiments, the durable polymer comprises a thermoset material. Example biocomaptible durable polymers include, but are not limited to, polystyrenes acrylates, epoxies. In some embodiments, the durable polymer comprises at least one of a polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, celluiosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof. The stent may be thin to be a base for the polymer to build upon, and the polymer itself may provide the radial strength and durability to withstand the forces encountered in the body, including but not limited to internal forces from blood flow, and external forces, such as may be encountered in peripheral vessels and other body lumens. The coated stents provided herein may be peripheral stents which may be delivered to vessels not protected by the rib cage and which may need to flex or withstand external forces without plastic deformation of the stent and without breaking struts of the stent. The coatings and coating methods provided herein provide substantial protection from these by establishing a multi-layer coating which can be bioabsorbable or durable or a combination thereof, and which can both deliver drugs and provide elasticity and radial strength for the vessel in which it is delivered.

In some embodiments, the forming the coating comprises depositing a first polymer layer, depositing a first drug layer, depositing a second polymer layer, depositing a second drug layer and depositing a third polymer layer. In some embodiments, the forming the coating comprises depositing a plurality of layers on said stent to form said coated stent. In some embodiments, the drug and polymer are in the same layer; in separate layers or form overlapping layers. In some embodiments, forming the coating comprises depositing at least 4 or more layers. In some embodiments, forming the coating comprises depositing 10, 20, 50, or 100 layers. In some embodiments, forming the coating comprises depositing at least one of: at least 10, at least 20, at least 50, and at least 100 layers. In some embodiments, forming the coating comprises depositing alternate drug and polymer layers. In some embodiments, forming the coating comprises depositing drug layers that are substantially free of polymer and the polymer layers are substantially free of drug.

In some embodiments forming the coating comprises depositing a fiber reinforcement on the stent. The fiber reinforcement may comprise a natural or a synthetic fiber. Examples of the fiber reinforcement may include any biocompatible fiber known in the art. This may, for non-limiting example, include any reinforcing fiber from silk to catgut to polymers (as described elsewhere herein) to olefins to acrylates. The fiber may be deposited according to methods disclosed herein, including by RESS. The concentration for a reinforcing fiber that is or comprises a polymer may be any concentration of the fiber forming polymer from 5 to 50 miligrams per milliliter and deposited according to the RESS process. For exmaple, methods of depositing the fiber may comprise and/or adapt methods described in Levit, et al., “Supercritical CO2 Assisted Electrospinning” J. of Supercritical Fluids, 329-333, Vol 31, Issue 3, (November 2004). In some embodiments, the fiber reinforcement is deposited on the stent in dry form. In some embodiments, depositing the fiber reinforcement on the stent meants to deposit the fiber reinforcement on another element of the coating (i.e. the pharmaceutical agent, the polymer, and/or another coating element). The fiber reinforcement need not be deposited directly on the stent in order to be deposited on the stent as part of the coating. The fiber reinforcement may be a part of another coating layer, such as a polymer layer or an active agent layer. The fiber may comprise a length to diameter ratio of at least 3:1, in some embodiments. The fiber may comprise lengths of at least 200 nanometers. The fiber may comprise lengths of up to 5 micrometers in certain embodiments. The fiber may comprise lengths of 200 nanometers to 5 micrometers, in some embodiments.

In some embodiments, the stent comprises at least one of stainless steel, a cobalt-chromium alloy, tantalum, platinum, Nitinol™, gold, a NiTi alloy, and a thermoplastic polymer. In some embodiments, stent is formed from a metal alloy. In some embodiments, the stent is capable of retaining its expanded condition upon the expansion thereof. In some embodiments, the stent is formed from a material that plastically deforms when subjected to at least 4 atmospheres of pressure. In some embodiments, the stent is formed from a material that is capable of self-expansion in the body lumen. In some embodiments, the stent is formed from a super-elastic metal alloy which transforms from an austenitic state to a martensitic state in the body lumen. In some embodiments, the stent is formed from a super-elastic metal alloy that is capable of deformation from a martensitic state to an austenitic state when the stent is mounted on a catheter. In some embodiments, the stent exhibits linear pseudoelasticity when stressed. In some embodiments, the stent is formed from a super-elastic metal alloy having a transformation temperature greater than a mammalian body temperature.

In some embodiments, at least one of the stent and the polymer is formed of a radiopaque material. In some embodiments, the stent comprises at least one of: iridium, platinum, gold, rhenium, tungsten, palladium, rhodium, tantalum, silver, ruthenium, chromium, iron, cobalt, vanadium, manganese, boron, copper, aluminum, niobium, zirconium, and hafnium.

In some embodiments, the method comprises forming a silane layer on a stent, and covalently attaching heparin to the silane layer. In some embodiments, the coated stent comprises a silane layer on a stent, and heparin attached to the silane layer. In some embodiments, onset of heparin anti-coagulant activity is obtained at week 3 or later. In some embodiments, heparin anti-coagulant activity remains at an effective level at least 90 days after onset of heparin activity. In some embodiments, heparin anti-coagulant activity remains at an effective level at least 120 days after onset of heparin activity. In some embodiments, heparin anti-coagulant activity remains at an effective level at least 200 days after onset of heparin activity.

In some embodiments, the polymer is 50/50 PLGA.

In some embodiments, at least part of said drug forms a phase separate from one or more phases formed by said polymer.

In some embodiments, the drug is at least 50% crystalline. In some embodiments, the drug is at least 75% crystalline. In some embodiments, the drug is at least 90% crystalline. In some embodiments, the drug is at least 95% crystalline. In some embodiments, the drug is at least 99% crystalline.

In some embodiments, the polymer is a mixture of two or more polymers. In some embodiments, the mixture of polymers forms a continuous film around particles of drug. In some embodiments, the two or more polymers are intimately mixed. In some embodiments, the mixture comprises no single polymer domain larger than about 20 nm. In some embodiments, each polymer in said mixture comprises a discrete phase. In some embodiments, the discrete phases formed by said polymers in said mixture are larger than about 10 nm. In some embodiments, the discrete phases formed by said polymers in said mixture are larger than about 50 nm.

Peripheral stent delivery sites are, typically (although not always), larger in diameter as compared to a coronary stent delivery site. Thus the stents delivered to that location need to be larger in diameter. Nevertheless, as a minimally invasive techinique, the peripheral stent also needs to be collapsed (and/or crimped) to a small diameter for delivery to the site, then expanded to a final diameter. Coating a stent having highter ratios of collapsed state to expanded state as compared to a coronary stent presents new challenges since the coating must withstand the expansion ratio without substantial cracking, tearing, and creation of other coating defects that might alter the elution of the drug from the coating into the vessel. The coatings (on the coated stents) and methods provided herein can address these defects by providing a way to coat the stents that is substantially conformal to the stent even in the expanded state. In some embodiments, the coated stent in the expanded state is at least about 99.99% free of coating defects. In some embodiments, the coated stent in the expanded state is at least about 99.99% free of coating defects. In some embodiments, the coated stent in the expanded state is at least about 99.9% free of coating defects. In some embodiments, the coated stent in the expanded state is at least about 99.0% free of coating defects. In some embodiments, the coated stent in the expanded state is at least about 98% free of coating defects. In some embodiments, the coated stent in the expanded state is at least about 97% free of coating defects. In some embodiments, the coated stent in the expanded state is at least about 95% free of coating defects. In some embodiments, the coated stent in the expanded state is at least about 94% free of coating defects. In some embodiments, the coated stent in the expanded state is at least about 93% free of coating defects. In some embodiments, the coated stent in the expanded state is at least about 92% free of coating defects. In some embodiments, the coated stent in the expanded state is at least about 90% free of coating defects. In some embodiments, the coated stent in the expanded state is at least about 85% free of coating defects. In some embodiments, the coated stent in the expanded state is at least about 80% free of coating defects. In some embodiments, the coated stent in the expanded state is at least about 75% free of coating defects. “About” when referring to coating defects, means plus or minus 0.01%-0.10%, plus or minus 0.1%-0.5%, plus or minus 0.5% to 1%, or plus or minus 1% to 5%. Coating defects may include at least one of cracks, bubbles, bare spots, bald spots, flaps, lifted coating, webs, and other visual defects.

In some embodiments, forming coating is done when the stent is in a collapsed state. In some embodiments, the coated stent has a radial expansion ratio of about 1 in a collapsed state up to about 3.0 in the expanded state. In some embodiments, the coated stent has a radial expansion ratio of about 1 in a collapsed state up to about 4.0 in the expanded state. In some embodiments, the coated stent has a radial expansion ratio of about 1 in a collapsed state up to about 5.0 in the expanded state. In some embodiments, the coated stent has a radial expansion ratio of about 1 in a collapsed state up to about 6.0 in the expanded state. In some embodiments, the coated stent has a radial expansion ratio of about 1 in a collapsed state to over about 3.0 in the expanded state. In some embodiments, the coated stent has a radial expansion ratio of about 1 in a collapsed state to over about 4.0 in the expanded state.

In some embodiments, the stent is adapted for delivery to at least one of a peripheral artery, a peripheral vein, a carotid artery, a vein, an aorta, and a biliary duct. In some embodiments, the stent is adapted for delivery to a superficial femoral artery. The stent may be adapted for delivery to a tibial artery. The stent may be adapted for delivery to a renal artery. The stent may be adapted for delivery to an iliac artery. The stent may be adapted for delivery to a bifurcated vessel. The stent is adapted for delivery to a vessel having a side branch at an intended delivery site of the vessel. The stent is adapted for delivery to the side branch of the vessel.

### EXAMPLES

The following examples are provided to illustrate selected embodiments. They should not be considered as limiting the scope of the invention, but merely as being illustrative and representative thereof. For each example listed below, multiple analytical techniques may be provided. Any single technique of the multiple techniques listed may be sufficient to show the parameter and/or characteristic being tested, or any combination of techniques may be used to show such parameter and/or characteristic. Those skilled in the art will be familiar with a wide range of analytical techniques for the characterization of drug/polymer coatings. Techniques presented here, but not limited to, may be used to additionally and/or alternatively characterize specific properties of the coatings with variations and adjustments employed which would be obvious to those skilled in the art.

### Sample Preparation

Generally speaking, coatings on stents, on coupons, or samples prepared for in-vivo models are prepared as below. Nevertheless, modifications for a given analytical method are presented within the examples shown, and/or would be obvious to one having skill in the art. Thus, numerous variations, changes, and substitutions will now occur to those skilled in the art without departing from the invention. It should be understood that various alternatives to the embodiments of the invention described herein and examples provided may be employed in practicing the invention and showing the parameters and/or characteristics described.

### Coatings on Stents

Coated stents as described herein and/or made by a method disclosed herein are prepared. In some examples, the coated stents have a targeted thickness of ˜15 microns (˜5 microns of active agent). In some examples, the coating process is PDPDP (Polymer, sinter, Drug, Polymer, sinter, Drug, Polymer, sinter) using deposition of drug in dry powder form and deposition of polymer particles by RESS methods and equipment described herein. In the illustrations below, resulting coated stents may have a 3-layer coating comprising polymer (for example, PLGA) in the first layer, drug (for example, rapamycin) in a second layer and polymer in the third layer, where a portion of the third layer is substantially drug free (e.g. a sub-layer within the third layer having a thickness equal to a fraction of the thickness of the third layer). As described layer, the middle layer (or drug layer) may be overlapping with one or both first (polymer) and third (polymer) layer. The overlap between the drug layer and the polymer layers is defined by extension of polymer material into physical space largely occupied by the drug. The overlap between the drug and polymer layers may relate to partial packing of the drug particles during the formation of the drug layer. When crystal drug particles are deposited on top of the first polymer layer, voids and or gaps may remain between dry crystal particles. The voids and gaps are available to be occupied by particles deposited during the formation of the third (polymer) layer. Some of the particles from the third (polymer) layer may rest in the vicinity of drug particles in the second (drug) layer. When the sintering step is completed for the third (polymer) layer, the third polymer layer particles fuse to form a continuous film that forms the third (polymer) layer. In some embodiments, the third (polymer) layer however will have a portion along the longitudinal axis of the stent whereby the portion is free of contacts between polymer material and drug particles. The portion of the third layer that is substantially of contact with drug particles can be as thin as 1 nanometer.

Polymer-coated stents having coatings comprising polymer but no drug are made by a method disclosed herein and are prepared having a targeted thickness of, for example, ˜5 microns. An example coating process is PPP (PLGA, sinter, PLGA, sinter, PLGA, sinter) using RESS methods and equipment described herein. These polymer-coated stents may be used as control samples in some of the examples, infra.

In some examples, the stents are made of a cobalt-chromium alloy and are 5 to 50 mm in length, preferably 10-20 mm in length, with struts of thickness between 20 and 100 microns, preferably 50-70 microns, measuring from an abluminal surface to a luminal surface, or measuring from a side wall to a side wall. In some examples, the stent may be cut lengthwise and opened to lay flat be visualized and/or assayed using the particular analytical technique provided.

The coating may be removed (for example, for analysis of a coating band and/or coating on a strut, and/or coating on the abluminal surface of a flattened stent) by scraping the coating off using a scalpel, knife or other sharp tool. This coating may be sliced into sections which may be turned 90 degrees and visualized using the surface composition techniques presented herein or other techniques known in the art for surface composition analysis (or other characteristics, such as crystallinity, for example). In this way, what was an analysis of coating composition through a depth when the coating was on the stent or as removed from the stent (i.e. a depth from the abluminal surface of the coating to the surface of the removed coating that once contacted the strut or a portion thereof), becomes a surface analysis of the coating which can, for example, show the layers in the slice of coating, at much higher resolution. Coating removed from the stent may be treated the same way, and assayed, visualized, and/or characterized as presented herein using the techniques described and/or other techniques known to a person of skill in the art.

### Coatings on Coupons

In some examples, samples comprise coupons of glass, metal, e.g. cobalt-chromium, or another substance that are prepared with coatings as described herein, with a plurality of layers as described herein, and/or made by a method disclosed herein. In some examples, the coatings comprise polymer. In some examples, the coatings comprise polymer and active agent. In some examples, the coated coupons are prepared having a targeted thickness of ˜10 microns (with ˜5 microns of active agent), and have coating layers as described for the coated stent samples, infra.

Sample Preparation for In-Vivo Models

Devices comprising stents having coatings disclosed herein are implanted in the porcine coronary arteries of pigs (domestic swine, juvenile farm pigs, or Yucatan miniature swine). Porcine coronary stenting is exploited herein since such model yields results that are comparable to other investigations assaying neointimal hyperplasia in human subjects. The stents are expanded to a 1:1.1 balloon:artery ratio. At multiple time points, animals are euthanized (e.g. t=1 day, 7 days, 14 days, 21 days, and 28 days), the stents are explanted, and assayed.

Devices comprising stents having coatings disclosed herein alternatively are implanted in the common iliac arteries of New Zealand white rabbits. The stents are expanded to a 1:1.1 balloon:artery ratio. At multiple time points, animals are euthanized (e.g., t=1 day, 7 days, 14 days, 21 days, and 28 days), the stents are explanted, and assayed.

### Example 1

In this example illustrates embodiments that provide a coated coronary stent, comprising: a stent and a rapamycin-polymer coating wherein at least part of rapamycin is in crystalline form and the rapamycin-polymer coating comprises one or more resorbable polymers.

In these experiments two different polymers were employed:
Polymer A:—50:50 PLGA-Ester End Group, MW˜19 kD, degradation rate ˜1-2 months
Polymer B:—50:50 PLGA-Carboxylate End Group, MW˜10 kD, degradation rate ˜28 days

Metal Stents were Coated as Follows:
AS1: Polymer A/Rapamycin/Polymer A/Rapamycin/Polymer A
AS2: Polymer A/Rapamycin/Polymer A/Rapamycin/Polymer B
AS1 (B): Polymer B/Rapamycin/Polymer B/Rapamycin/Polymer B (also called AS1(213) elsewhere herein)
AS1b: Polymer A/Rapamycin/Polymer A/Rapamycin/Polymer A
AS2b: Polymer A/Rapamycin/Polymer A/Rapamycin/Polymer B

Average Coating Masses were as Follows:

| Stent | Average Rapamycin | Average Polymer | Average total Mass |
|---|---|---|---|
| Coating | (micrograms) | (micrograms) | (micrograms) |
| AS1 | 175 | 603 | 778 |
| AS2 | 153 | 717 | 870 |
| AS1(B) | 224 | 737 | 961 |
| AS1b | 171 | 322 | 493 |
| AS2b | 167 | 380 | 547 |

Elution results are illustrated in FIGS. 1-4 (see also Example 11)

### Example 2

### Crystallinity

The presence and or quantification of the Active agent crystallinity can be determined from a number of characterization methods known in the art, but not limited to, XRPD, vibrational spectroscopy (FTIR, NIR, Raman), polarized optical microscopy, calorimetry, thermal analysis and solid-state NMR.

X-Ray Diffraction to Determine the Presence and/or Quantification of Active Agent Crystallinity

Active agent and polymer coated proxy substrates are prepared using 316L stainless steel coupons for X-ray powder diffraction (XRPD) measurements to determine the presence of crystallinity of the active agent. The coating on the coupons is equivalent to the coating on the stents described herein. Coupons of other materials described herein, such as cobalt-chromium alloys, may be similarly prepared and tested. Likewise, substrates such as stents, or other medical devices described herein may be prepared and tested. Where a coated stent is tested, the stent may be cut lengthwise and opened to lay flat in a sample holder.

For example XRPD analyses are performed using an X-ray powder diffractometer (for example, a Bruker D8 Advance X-ray diffractometer) using Cu Kα radiation. Diffractograms are typically collected between 2 and 40 degrees 2 theta. Where required low background XRPD sample holders are employed to minimize background noise.

The diffractograms of the deposited active agent are compared with diffractograms of known crystallized active agents, for example micronized crystalline sirolimus in powder form. XRPD patterns of crystalline forms show strong diffraction peaks whereas amorphous show diffuse and non-distinct patterns. Crystallinity is shown in arbitrary Intensity units.

A related analytical technique which may also be used to provide crystallinity detection is wide angle scattering of radiation (e.g.; Wide Anle X-ray Scattering or WAXS), for example, as described in F. Unger, et al., “Poly(ethylene carbonate): A thermoelastic and biodegradable biomaterial for drug eluting stent coatings?” Journal of Controlled Release, Volume 117, Issue 3, 312-321 (2007) for which the technique and variations of the technique specific to a particular sample would be obvious to one of skill in the art.

### Raman Spectroscopy

Raman spectroscopy, a vibrational spectroscopy technique, can be useful, for example, in chemical identification, characterization of molecular structures, effects of bonding, identification of solid state form, environment and stress on a sample. Raman spectra can be collected from a very small volume (<1 μm³); these spectra allow the identification of species present in that volume. Spatially resolved chemical information, by mapping or imaging, terms often used interchangeably, can be achieved by Raman microscopy.

Raman spectroscopy and other analytical techniques such as described in Balss, et al., “Quantitative spatial distribution of sirolimus and polymers in drug-eluting stents using confocal Raman microscopy” J. of Biomedical Materials Research Part A, 258-270 (2007), incorporated in its entirety herein by reference, and/or described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

For example, to test a sample using Raman microscopy and in particular confocal Raman microscopy, it is understood that to get appropriate Raman high resolution spectra sufficient acquisition time, laser power, laser wavelength, sample step size and microscope objective need to be optimized. For example a sample (a coated stent) is prepared as described herein. Alternatively, a coated coupon could be tested in this method. Maps are taken on the coating using Raman microscopy. A WITec CRM 200 scanning confocal Raman microscope using a Nd:YAG laser at 532 nm is applied in the Raman imaging mode. The laser light is focused upon the sample using a 100×dry objective (numerical aperture 0.90), and the finely focused laser spot is scanned into the sample. As the laser scans the sample, over each 0.33 micron interval a Raman spectrum with high signal to noise is collected using 0.3 seconds of integration time. Each confocal cross-sectional image of the coatings displays a region 70 μm wide by 10 μm deep, and results from the gathering of 6300 spectra with a total imaging time of 32 min.

Multivariate analysis using reference spectra from samples of rapamycin (amorphous and crystalline) and polymer are used to deconvolve the spectral data sets, to provide chemical maps of the distribution.

### Infrared (IR) Spectroscopy for In-Vitro Testing

Infrared (IR) Spectroscopy such as FTIR and ATR-IR are well utilized techniques that can be applied to show, for example, the quantitative drug content, the distribution of the drug in the sample coating, the quantitative polymer content in the coating, and the distribution of polymer in the coating. Infrared (IR) Spectroscopy such as FTIR and ATR-IR can similarly be used to show, for example, drug crystallinity. The following table (Table 1) lists the typical IR materials for various applications. These IR materials are used for IR windows, diluents or ATR crystals.

TABLE 1

| MATERIAL | NACL | KBR | CSI | AGCL | GE | ZNSE | DIAMOND |
|---|---|---|---|---|---|---|---|
| Transmission | 40,000~625 | 40,000~400 | 40,000~200 | 25,000~360 | 5,500~625 | 20,000~454 | 40,000~2,500 & |
| range (cm−1) | | | | | | | 1667-33 |
| Water sol | 35.7 | 53.5 | 44.4 | Insol. | Insol. | Insol. | Insol. |
| (g/100 g, | | | | | | | |
| 25 C.) | | | | | | | |
| Attacking | Wet | Wet | Wet | Ammonium | H2SO4, | Acids, strong | K2Cr2Os, |
| materials | Solvents | Solvents | Solvents | Salts | aqua regin | alkalies, | conc. |
| | | | | | | chlorinated | H2SO4 |
| | | | | | | solvents | |

In one test, a coupon of crystalline ZnSe is coated by the processes described herein, creating a PDPDP (Polymer, Drug, Polymer, Drug, Polymer) layered coating that is about 10 microns thick. The coated coupon is analyzed using FTIR. The resulting spectrum shows crystalline drug as determined by comparison to the spectrum obtained for the crystalline form of a drug standard (i.e. a reference spectrum).

Differential Scanning calorimetry (DSC)

DSC can provide qualitative evidence of the crystallinity of the drug (e.g. rapamycin) using standard DSC techniques obvious to one of skilled in the art. Crystalline melt can be shown using this analytical method (e.g. rapamycin crystalline melting—at about 185 decrees C to 200 degrees C., and having a heat of fusion at or about 46.8 J/g). The heat of fusion decreases with the percent crystallinity. Thus, the degree of crystallinity could be determined relative to a pure sample, or versus a calibration curve created from a sample of amorphous drug spiked and tested by DSC with known amounts of crystalline drug. Presence (at least) of crystalline drug on a stent could be measured by removing (scraping or stripping) some drug from the stent and testing the coating using the DSC equipment for determining the melting temperature and the heat of fusion of the sample as compared to a known standard and/or standard curve.

### Example 3

### Determination of Bioabsorbability/Bioresorbability/Dissolution Rate of a Polymer Coating a Device

### Gel Permeation Chromatography In-Vivo Weight Loss Determination

Standard methods known in the art can be applied to determine polymer weight loss, for example gel permeation chromatography and other analytical techniques such as described in Jackson et al., “Characterization of perivascular poly(lactic-co-glycolic acid) films containing paclitaxel” Int. J. of Pharmaceutics, 283:97-109 (2004), incorporated in its entirety herein by reference.

For example rabbit in vivo models as described above are euthanized at multiple time points (t=1 day, 2 days, 4 days, 7 days, 14 days, 21 days, 28 days, 35 days n=5 per time point). Alternatively, pig in vivo models as described above are euthanized at multiple time points (t=1 day, 2 days, 4 days, 7 days, 14 days, 21 days, 28 days, 35 days n=5 per time point). The stents are explanted, and dried down at 30° C. under a stream of gas to complete dryness. A stent that has not been implanted in the animal is used as a control for no loss of polymer.

The remaining polymer on the explanted stents is removed using a solubilizing solvent (for example chloroform). The solutions containing the released polymers for each time point are filtered. Subsequent GPC analysis is used for quantification of the amount of polymer remaining in the stent at each explant time point. The system, for example, comprises a Shimadzu LC-10 AD HPLC pump, a Shimadzu RID-6A refractive index detector coupled to a 50 Å Hewlett Packard Pl-Gel column. The polymer components are detected by refractive index detection and the peak areas are used to determine the amount of polymer remaining in the stents at the explant time point. A calibration graph of log molecular weight versus retention time is established for the 50A Pl-Gel column using polystyrene standards with molecular weights of 300, 600, 1.4 k, 9 k, 20 k, and 30 k g/mol. The decreases in the polymer peak areas on the subsequent time points of the study are expressed as weight percentages relative to the 0 day stent.

### Gel Permeation Chromatography In-Vitro Testing

Gel Permeation Chromatography (GPC) can also be used to quantify the bioabsorbability/bioresorbability, dissolution rate, and/or biodegrability of the polymer coating. The in vitro assay is a degradation test where the concentration and molecular weights of the polymers can be assessed when released from the stents in an aqueous solution that mimics physiological surroundings. See for example, Jackson et al., “Characterization of perivascular poly(lactic-co-glycolic acid) films containing paclitaxel” Int. J. of Pharmaceutics, 283:97-109 (2004), incorporated in its entirety herein by reference.

For example Stents (n=15) described herein are expanded and then placed in a solution of 1.5 ml solution of phosphate buffered saline (pH=7.4) with 0.05% wt of Tween20, or in the alternative 10 mM Tris, 0.4 wt. % SDS, pH 7.4, in a 37° C. bath with bath rotation at 70 rpm. Alternatively, a coated coupon could be tested in this method. The solution is then collected at the following time points: 0 min., 15 min., 30 min., 1 hr, 2 hr, 4 hr, 6 hr, 8 hr, 12 hr, 16 hr, 20 hr, 24 hr, 30 hr, 36 hr, 48 hr, and daily up to 70 days, for example. The solution is replaced at least at each time point, and/or periodically (e.g. every four hours, daily, weekly, or longer for later time points) to prevent saturation, the removed solution is collected, saved, and assayed. The solutions containing the released polymers for each time point are filtered to reduce clogging the GPC system. For time points over 4 hours, the multiple collected solutions are pooled together for liquid extraction.

1 ml Chloroform is added to the phosphate buffered saline solutions and shaken to extract the released polymers from the aqueous phase. The chloroform phase is then collected for assay via GPC.

The system comprises a Shimadzu LC-10 AD HPLC pump, a Shimadzu RID-6A refractive index (RI) detector coupled to a 50A Hewlett Packard Pl-Gel column. The mobile phase is chloroform with a flow rate of 1 mL/min. The injection volume of the polymer sample is 100 μL of a polymer concentration. The samples are run for 20 minutes at an ambient temperature.

For determination of the released polymer concentrations at each time point, quantitative calibration graphs are first made using solutions containing known concentrations of each polymer in chloroform. Stock solutions containing each polymer in 0-5 mg/ml concentration range are first analyzed by GPC and peak areas are used to create separate calibration curves for each polymer.

For polymer degradation studies, a calibration graph of log molecular weight versus retention time is established for a 50 Å Pl-Gel column (Hewlett Packard) using polystyrene standards with molecular weights of 300, 600, 1.4 k, 9 k, 20 k, and 30 k g/mol. In the alternative, a Multi angle light scattering (MALS) detector may be fitted to directly assess the molecular weight of the polymers without the need of polystyrene standards.

To perform an accelerated in-vitro dissolution of the bioresorbable polymers, a protocol is adapted from ISO Standard 13781 “Poly(L-lactide) resides and fabricated an accelerated froms for surgical implants—in vitro degradation testing” (1997), incorporated in its entirety herein by reference. Briefly, elution buffer comprising 18% v/v of a stock solution of 0.067 mol/L KH₂PO₄ and 82% v/v of a stock solution of 0.067 mol/L Na₂HPO₄ with a pH of 7.4 is used. Stents described herein are expanded and then placed in 1.5 ml solution of this accelerated elution in a 70° C. bath with rotation at 70 rpm. The solutions are then collected at the following time points: 0 min., 15 min., 30 min., 1 hr, 2 hr, 4 hr, 6 hr, 8 hr, 12 hr, 16 hr, 20 hr, 24 hr, 30 hr, 36 hr and 48 hr. Fresh accelerated elution buffer are added periodically every two hours to replace the incubated buffers that are collected and saved in order to prevent saturation. The solutions containing the released polymers for each time point are filtered to reduce clogging the GPC system. For time points over 2 hours, the multiple collected solutions are pooled together for liquid extraction by chloroform. Chloroform extraction and GPC analysis is performed in the manner described above.

Scanning Electron Microscopy (SEM) with Focused Ion Beam (FIB) Milling In-Vitro Testing

Focused ion beam FIB is a tool that allows precise site-specific sectioning, milling and depositing of materials. FIB can be used in conjunction with SEM, at ambient or cryo conditions, to produce in-situ sectioning followed by high-resolution imaging. FIB-SEM can produce a cross-sectional image of the polymer layers on the stent. The image can be used to quantitate the thickness of the layers to reveal rate of bioresorbability of single or multiple polymers as well as show whether there is uniformity of the layer thickness at manufacture and at time points after stenting (or after in-vitro elution at various time points).

For example, testing is performed at multiple time points. Stents are removed from the elution media and dried, the dried stent is visualized using FIB-SEM for changes in the coating. Alternatively, a coated coupon could be tested in this method.

Stents (n=15) described herein are expanded and then placed in 1.5 ml solution of phosphate buffered saline (pH=7.4) with 0.05% wt of Tween20 in a 37° C. bath with bath rotation at 70 rpm. Alternatively, a coated coupon could be tested in this method. The phosphate buffered saline solution is periodically replaced with fresh solution at each time point and/or every four hours to prevent saturation. The stents are collected at the following time points: 30 min, 1 hr, 2 hr, 4 hr, 6 hr, 8, hr, 12 hr, 16 hr, 20 hr, 24 hr, 30 hr, 36 hr, 48 hr, 60 h and 72 h. The stents are dried down at 30° C. under a stream of gas to complete dryness. A stent that not been subjected to these conditions is used as a t=0 control.

A FEI Dual Beam Strata 235 FIB/SEM system is a combination of a finely focused Ga ion beam (FIB) accelerated by 30 kV with a field emission electron beam in a scanning electron microscope instrument and is used for imaging and sectioning the stents. Both beams focus at the same point of the sample with a probe diameter less than 10 nm. The FIB can also produce thinned down sections for TEM analysis.

To prevent damaging the surface of the stent with incident ions, a Pt coating is first deposited via electron beam assisted deposition and ion beam deposition prior to FIB sectioning. For FIB sectioning, the Ga ion beam is accelerated to 30 kV and the sectioning process is about 2 h in duration. Completion of the FIB sectioning allows one to observe and quantify by SEM the thickness of the polymer layers that are left on the stent as they are absorbed.

### Raman Spectroscopy In-Vitro Testing

As discussed in example 2, Raman spectroscopy can be applied to characterize the chemical structure and relative concentrations of drug and polymer coatings. This can also be applied to characterize in-vitro tested polymer coatings on stents or other substrates.

For example, confocal Raman Spectroscopy/microscopy can be used to characterize the relative drug to polymer ratio at the outer ˜1 μm of the coated surface as a function of time exposed to elution media. In addition confocal Raman x-z or z (maps or line scans) microscopy can be applied to characterize the relative drug to polymer ratio as a function of depth at time t after exposure to elution media.

For example a sample (a coated stent) is prepared as described herein and placed in elution media (e.g., 10 mM tris(hydroxymethyl)aminomethane (Tris), 0.4 wt. % Sodium dodecyl sulphate (SDS), pH 7.4 or 1.5 ml solution of phosphate buffered saline (pH=7.4) with 0.05% wt of Tween20) in a 37° C. bath with bath rotation at 70 rpm. Confocal Raman Images are taken on the coating before elution. At at least four elution time points within a 48 day interval, (e.g. 0 min., 15 min., 30 min., 1 hr, 2 hr, 4 hr, 6 hr, 8, hr, 12 hr, 16 hr, 20 hr, 24 hr, 30 hr, 36 hr and 48 hr) the sample is removed from the elution, and dried (for example, in a stream of nitrogen). The dried stent is visualized using Raman Spectroscopy for changes in coating. Alternatively, a coated coupon could be tested in this method. After analysis, each is returned to the buffer for further elution.

Raman spectroscopy and other analytical techniques such as described in Balss, et al., “Quantitative spatial distribution of sirolimus and polymers in drug-eluting stents using confocal Raman microscopy” J. of Biomedical Materials Research Part A, 258-270 (2007), incorporated in its entirety herein by reference, and/or described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

For example a WITec CRM 200 scanning confocal Raman microscope using a Nd:YAG laser at 532 nm is applied in the Raman imaging mode to generate an x-z map. The sample is placed upon a piezoelectrically driven table, the laser light is focused upon the sample using a 100×dry objective (numerical aperture 0.90), and the finely focused laser spot is scanned into the sample. As the laser scans the sample, over each 0.33 micron interval a Raman spectrum with high signal to noise is collected using 0.3 Seconds of integration time. Each confocal cross-sectional image of the coatings displays a region 70 μm wide by 10 μm deep, and results from the gathering of 6300 spectra with a total imaging time of 32 min.

### SEM-In-Vitro Testing

Testing is performed at multiple time points (e.g. 0 min., 15 min., 30 min., 1 hr, 2 hr, 4 hr, 6 hr, 8, hr, 12 hr, 16 hr, 20 hr, 24 hr, 30 hr, 36 hr and 48 hr). Stents are removed from the elution media (described supra) and dried at these time points. The dried stent is visualized using SEM for changes in coating.

For example the samples are observed by SEM using a Hitachi S-4800 with an accelerating voltage of 800V. Various magnifications are used to evaluate the coating integrity, especially at high strain regions. Change in coating over time is evaluated to visualize the bioabsorption of the polymer over time.

### X-Ray Photoelectron Spectroscopy (XPS)—In-Vitro Testing

XPS can be used to quantitatively determine elemental species and chemical bonding environments at the outer 5-10 nm of sample surface. The technique can be operated in spectroscopy or imaging mode. When combined with a sputtering source, XPS can be utilized to give depth profiling chemical characterization.

XPS testing can be used to characterize the drug to polymer ratio at the very surface of the coating of a sample. Additionally XPS testing can be run in time lapse to detect changes in composition. Thus, in one test, samples are tested using XPS at multiple time points (e.g. 0 min., 15 min., 30 min., 1 hr, 2 hr, 4 hr, 6 hr, 8, hr, 12 hr, 16 hr, 20 hr, 24 hr, 30 hr, 36 hr and 48 hr). Stents are removed from the elution media (e.g., 10 mM Tris, 0.4 wt. % SDS, pH 7.4 or 1.5 ml solution of phosphate buffered saline (pH=7.4) with 0.05% wt of Tween20) in a 37° C. bath with rotation at 70 rpm and dried at these time points.

XPS (ESCA) and other analytical techniques such as described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

For example, XPS analysis is performed using a Physical Electronics Quantum 2000 Scanning ESCA. The monochromatic Al Kα source is operated at 15 kV with a power of 4.5 W. The analysis is performed at a 45° take off angle. Three measurements are taken along the length of each stent with the analysis area ˜20 microns in diameter. Low energy electron and Ar⁺ ion floods are used for charge compensation.

### Time of Flight Secondary Ion Mass Spectrometers (TOF-SIMS)

TOF-SIMS can be used to determine molecular species at the outer 1-2 nm of sample surface when operated under static conditions. The technique can be operated in spectroscopy or imaging mode at high spatial resolution. When operated under dynamic experimental conditions, known in the art, depth profiling chemical characterization can be achieved.

TOF-SIMS testing can be used to characterize the presence of polymer and or drug at uppermost surface of the coating of a sample. Additionally TOF-SIMS testing can be run in time lapse to detect changes in composition. Thus, in one test, samples are tested using TOF-SIMS at multiple time points (e.g., 0 min., 15 min., 30 min., 1 hr, 2 hr, 4 hr, 6 hr, 8, hr, 12 hr, 16 hr, 20 hr, 24 hr, 30 hr, 36 hr and 48 hr). Stents are removed from the elution media (e.g. 10 mM Tris, 0.4 wt. % SDS, pH 7.4 or 1.5 ml solution of phosphate buffered saline (pH=7.4) with 0.05% wt of Tween20) in a 37° C. bath with rotation at 70 rpm and dried at these time points.

For example, to analyze the uppermost surface only, static conditions (for example a ToF-SIMS IV (IonToF, Munster)) using a 25 Kv Bi⁺⁺ primary ion source maintained below 10¹² ions per cm² is used. Where necessary a low energy electron flood gun (0.6 nA DC) is used to charge compensate insulating samples.

Cluster Secondary Ion Mass Spectrometry, may be employed for depth profiling as described Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference.

For example, a stent as described herein is obtained. The stent is prepared for SIMS analysis by cutting it longitudinally and opening it up with tweezers. The stent is then pressed into multiple layers of indium foil with the outer diameter facing outward.

TOF-SIMS depth profiling experiments are performed using an Ion-TOF IV instrument equipped with both Bi and SF5+ primary ion beam cluster sources. Sputter depth profiling is performed in the dual-beam mode, while preserving the chemical integrity of the sample. For example, the analysis source is a pulsed, 25-keV bismuth cluster ion source, which bombarded the surface at an incident angle of 45° to the surface normal. The target current is maintained at ˜0.3 pÅ (+10%) pulsed current with a raster size of 200 micron×200 micron for all experiments. Both positive and negative secondary ions are extracted from the sample into a reflectron-type time-of-flight mass spectrometer. The secondary ions are then detected by a microchannel plate detector with a post-acceleration energy of 10 kV. A low-energy electron flood gun is utilized for charge neutralization in the analysis mode.

The sputter source used is a 5-keV SF5+ cluster source also operated at an incident angle of 45° to the surface normal. For thin model samples on Si, the SF5+ current is maintained at ˜2.7 nÅ with a 750 micron×750 micron raster. For the thick samples on coupons and for the samples on stents, the current is maintained at 6 nA with a 500 micron×500 micron raster. All primary beam currents are measured with a Faraday cup both prior to and after depth profiling.

All depth profiles are acquired in the noninterlaced mode with a 5-ms pause between sputtering and analysis. Each spectrum is averaged over a 7.37 second time period. The analysis is immediately followed by 15 seconds of SF₅⁺ sputtering. For depth profiles of the surface and subsurface regions only, the sputtering time was decreased to 1 second for the 5% active agent sample and 2 seconds for both the 25% and 50% active agent samples.

Temperature-controlled depth profiles are obtained using a variable-temperature stage with Eurotherm Controls temperature controller and IPSG V3.08 software. Samples are first placed into the analysis chamber at room temperature. The samples are brought to the desired temperature under ultra high-vacuum conditions and are allowed to stabilize for 1 minute prior to analysis. All depth profiling experiments are performed at −100 degrees C. and 25 degrees C.

### Infrared (IR) Spectroscopy for In-Vitro Testing

Infrared (IR) Spectroscopy such as, but not limited to, FTIR, ATR-IR and micro ATR-IR are well utilized techniques that can be applied to show the quantitative polymer content in the coating, and the distribution of polymer in the coating.

For example using FTIR, a coupon of crystalline ZnSe is coated by the processes described herein, creating a PDPDP (Polymer, Drug, Polymer, Drug, Polymer) layered coating that is about 10 microns thick. At time=0 and at at least four elution time points within a 48 day interval (e.g., 0 min., 15 min., 30 min., 1 hr, 2 hr, 4 hr, 6 hr, 8, hr, 12 hr, 16 hr, 20 hr, 24 hr, 30 hr, 36 hr and 48 hr), the sample (coated crystal) was tested by FTIR for polymer content. The sample was placed in an elution media (e.g. 10 mM Tris, 0.4 wt. % SDS, pH 7.4 or 1.5 ml solution of phosphate buffered saline (pH=7.4) with 0.05% wt of Tween20) in a 37° C. bath with bath rotation at 70 rpm and at each time point, the sample is removed from the elution media and dried (e.g. in a stream of nitrogen). FTIR spectrometry was used to quantify the polymer on the sample. After analysis, each is returned to the buffer for further elution.

In another example using FTIR, sample elution media at each time point was tested for polymer content. In this example, a coated stent was prepared that was coated by the processes described herein, creating a PDPDP (Polymer, Drug, Polymer, Drug, Polymer) layered coating that is about 10 microns thick. The coated stent was placed in an elution media (e.g. 10 mM Tris, 0.4 wt. % SDS, pH 7.4 or 1.5 ml solution of phosphate buffered saline (pH=7.4) with 0.05% wt of Tween20) in a 37° C. bath with rotation at 70 rpm. and at each time point (e.g., 0 min., 15 min., 30 min., 1 hr, 2 hr, 4 hr, 6 hr, 8, hr, 12 hr, 16 hr, 20 hr, 24 hr, 30 hr, 36 hr and 48 hr), a sample of the elution media is removed and dried onto a crystalline ZnSe window (e.g. in a stream of nitrogen). At each elution time point, the sample elution media was tested by FTIR for polymer content.

### Atomic Force Microscopy (AFM)

AFM is a high resolution surface characterization technique. AFM is used in the art to provide topographical imaging, in addition when employed in Tapping Mode™ can image material and or chemical properties of the surface. The technique can be used under ambient, solution, humidified or temperature controlled conditions. Other modes of operation are well known and can be readily employed here by those skilled in the art. The AFM topography images can be run in time-lapse to characterize the surface as a function of elution time. Three-dimensionally rendered images show the surface of a coated stent, which can show holes or voids of the coating which may occur as the polymer is absorbed and the drug is eluted over time.

A stent as described herein is obtained. AFM is used to determine the drug polymer distribution. AFM may be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

For example a multi-mode AFM (Digital Instruments/Veeco Metrology, Santa Barbara, Calif.) controlled with Nanoscope IIIa and NanoScope Extender electronics is used. Samples are examined in the dry state using AFM before elution of the drug (e.g. rapamycin). Samples are also examined at select time points through a elution period (e.g. 48 hours) by using an AFM probe-tip and flow-through stage built to permit analysis of wet samples. The wet samples are examined in the presence of the same elution medium used for in-vitro kinetic drug release analysis (e.g. PBS-Tween20, or 10 mM Tris, 0.4 wt. % SDS, pH 7.4). Saturation of the solution is prevented by frequent exchanges of the release medium with several volumes of fresh medium. TappingMode™ AFM imaging may be used to show topography (a real-space projection of the coating surface microstructure) and phase-angle changes of the AFM over the sample area to contrast differences in the material and physical structure.

### Nano X-Ray Computer Tomography

Another technique that may be used to view the physical structure of a device in 3-D is Nano X-Ray Computer Tomography (e.g. such as made by SkyScan), which could be used in an elution test and/or bioabsorbability test, as described herein to show the physical structure of the coating remaining on stents at each time point, as compared to a scan prior to elution/bioabsorbtion.

### pH Testing

The bioabsorbability of PLGA of a coated stent can be shown by testing the pH of an elution media (EtOH/PBS, for example) in which the coated stent is placed. Over time, a bioabsorbable PLGA coated stent (with or without the drug) will show a decreased pH until the PLGA is fully bioabsorbed by the elution media.

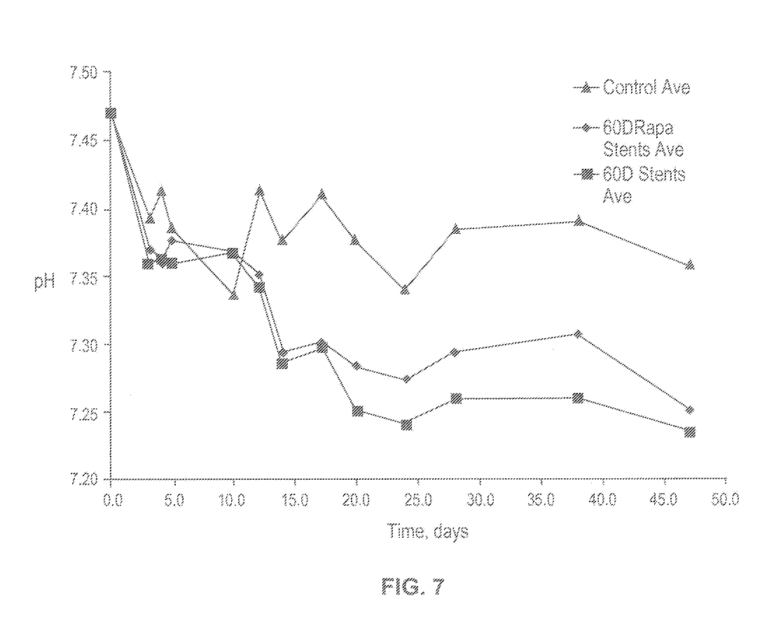

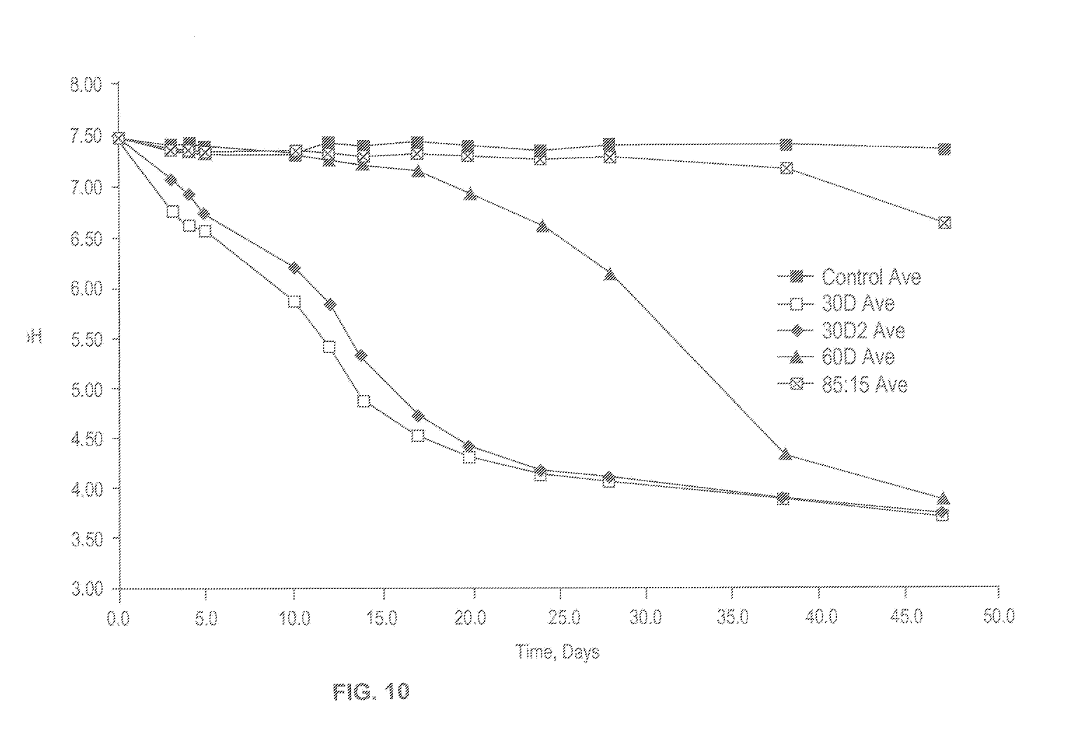

A test was performed using stents coated with PLGA alone, stents coated with PLGA and rapamycin, PLGA films, and PLGA films containing rapamycin. The samples were put in elution media of 20% EtOH/PBS at 37° C. The elution media was tested at mutliple intervals from 0 to 48 days. In FIGS. 7, 8, and 9, stents having coatings as provided herein were tested for pH over time according to this method. FIG. 10 shows results of the PLGA films (with and without rapamycin) tested according to this method. Control elution media was run in triplicate alongside the samples, and the results of this pH testing was averaged and is presented as “Control AVE” in each of the FIGS. 7-10.

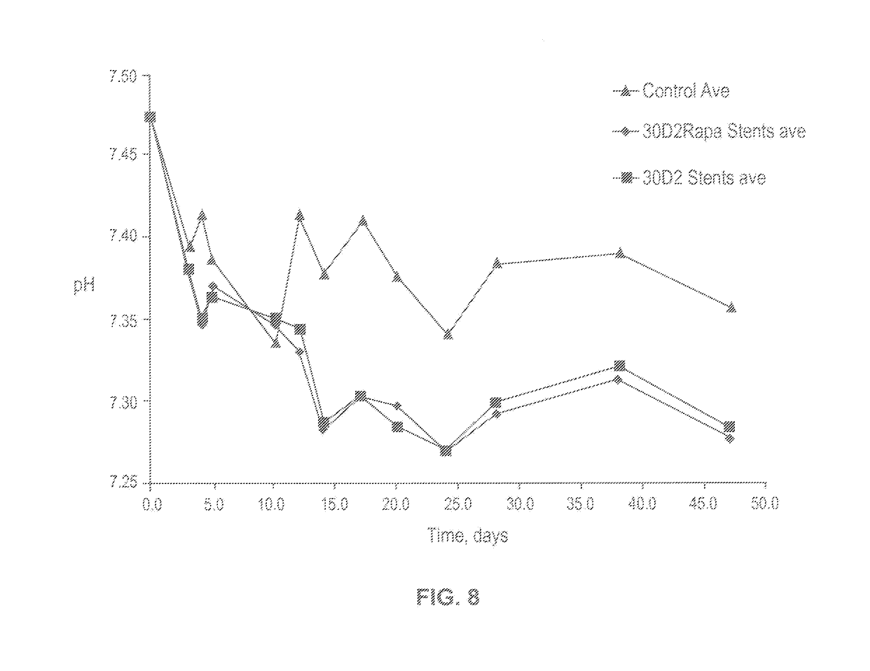

In FIG. 8, the “30D2Rapa Stents ave” line represents a stent having coating according to AS1(213) of Example 1 (PDPDP) with Polymer B (50:50 PLGA-Carboxylate end group, MW˜10 kD) and rapamycin, where the coating was removed from the stent and tested in triplicate for pH changes over time in the elution media, the average of which is presented. The “30D2 Stents ave” line represents a stent having coating of only Polymer B (50:50 PLGA-Carboxylate end group, MW˜10 kD) (no rapamycin), where the coating was removed from the stent and tested in triplicate for pH changes over time in the elution media, the average of which is presented.

In FIG. 7, the “60DRapa Stents ave” line represents a stent having coating according to AS1 of Example 1 (PDPDP) with Polymer A (50:50 PLGA-Ester end group, MW˜19 kD) and rapamycin, where the coating was removed from the stent and tested in triplicate for pH changes over time in the elution media, the average of which is presented. The “60D Stents ave” line represents a stent having coating of only Polymer A (50:50 PLGA-Ester end group, MW˜19 kD) (no rapamycin), where the coating was removed from the stent and tested in triplicate for pH changes over time in the elution media, the average of which is presented.

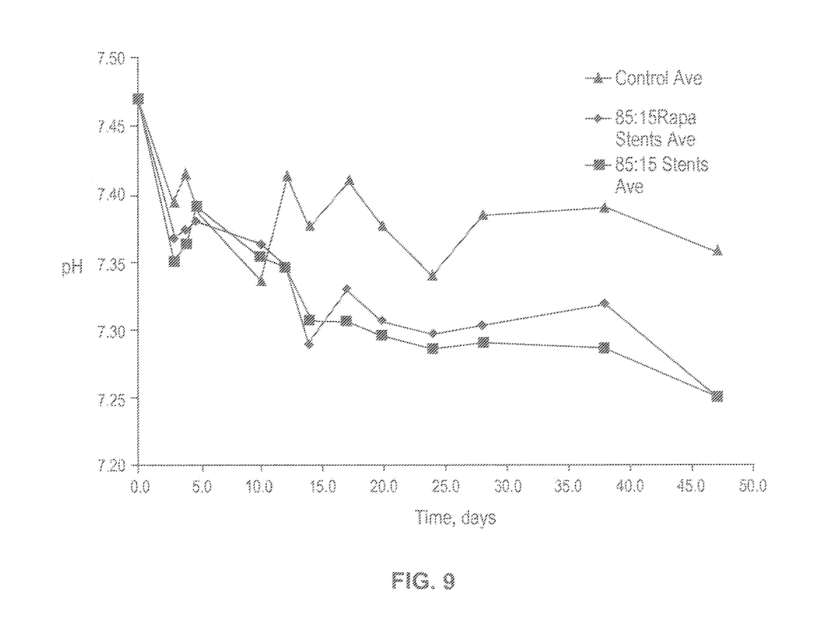

In FIG. 9, the “85:15 Rapa Stents ave” line represents a stent having coating according to PDPDP with a PLGA comprising 85% lactic acid, 15% glycolic acid, and rapamycin, where the coating was removed from the stent and tested in triplicate for pH changes over time in the elution media, the average of which is presented. The “85:15 Stents ave” line represents a stent having coating of only PLGA comprising 85% lactic acid, 15% glycolic acid (no rapamycin), where the coating was removed from the stent and tested in triplicate for pH changes over time in the elution media, the average of which is presented.

In FIG. 10, the “30D Ave” line represents a polymer film comprising Polymer B (50:50 PLGA-Carboxylate end group, MW˜10 kD) (no rapamycin), where the film was tested in triplicate for pH changes over time in the elution media, the average of which is presented.

The “30D2 Ave” line also represents a polymer film comprising Polymer B (50:50 PLGA-Carboxylate end group, MW˜10 kD) (no rapamycin), where the film was tested in triplicate for pH changes over time in the elution media, the average of which is presented. The “60D Ave” line represents a polymer film comprising Polymer A (50:50 PLGA-Ester end group, MW ˜19 kD) (no rapamycin), where the film was tested in triplicate for pH changes over time in the elution media, the average of which is presented. The “85:15 Ave” line represents a polymer film comprising PLGA comprising 85% lactic acid, 15% glycolic acid (no rapamycin), where the film was tested in triplicate for pH changes over time in the elution media, the average of which is presented. To create the polymer films in FIG. 10, the polymers were dissolved in methylene chloride, THF, and ethyl acetate. The films that were tested had the following average thicknesses and masses, 30D—152.4 um, 12.0 mg; 30D2—127.0 um, 11.9 mg; 60D—50.8 um, 12.4 mg; 85:15—127 um, 12.5 mg.

### Example 4

### Visualization of Polymer/Active Agent Layers Coating a Device

### Raman Spectroscopy

As discussed in example 2, Raman spectroscopy can be applied to characterize the chemical structure and relative concentrations of drug and polymer coatings. For example, confocal Raman Spectroscopy/microscopy can be used to characterize the relative drug to polymer ratio at the outer ˜1 μm of the coated surface. In addition confocal Raman x-z or z (maps or line scans) microscopy can be applied to characterize the relative drug to polymer ratio as a function of depth. Additionally cross-sectioned samples can be analysed. Raman spectroscopy and other analytical techniques such as described in Balss, et al., “Quantitative spatial distribution of sirolimus and polymers in drug-eluting stents using confocal Raman microscopy” J. of Biomedical Materials Research Part A, 258-270 (2007), incorporated in its entirety herein by reference, and/or described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

A sample (a coated stent) is prepared as described herein. Images are taken on the coating using Raman Spectroscopy. Alternatively, a coated coupon could be tested in this method. To test a sample using Raman microscopy and in particular confocal Raman microscopy, it is understood that to get appropriate Raman high resolution spectra sufficient acquisition time, laser power, laser wavelength, sample step size and microscope objective need to be optimized.

For example a WITec CRM 200 scanning confocal Raman microscope using a Nd:YAG laser at 532 nm is applied in the Raman imaging mode to give x-z maps. The sample is placed upon a piezoelectrically driven table, the laser light is focused upon the sample using a 100×dry objective (numerical aperture 0.90), and the finely focused laser spot is scanned into the sample. As the laser scans the sample, over each 0.33 micron interval a Raman spectrum with high signal to noise is collected using 0.3 Seconds of integration time. Each confocal cross-sectional image of the coatings displays a region 70 μm wide by 10 μm deep, and results from the gathering of 6300 spectra with a total imaging time of 32 min. Multivariate analysis using reference spectra from samples of rapamycin and polymer are used to deconvolve the spectral data sets, to provide chemical maps of the distribution.

In another test, spectral depth profiles (x-z maps) of samples are performed with a CRM200 microscope system from WITec Instruments Corporation (Savoy, Ill.). The instrument is equipped with a Nd:YAG frequency doubled laser (532 excitation), a single monochromator (Acton) employing a 600 groove/mm grating and a thermoelectrically cooled 1024 by 128 pixel array CCD camera (Andor Technology). The microscope is equipped with appropriate collection optics that include a holographic laser bandpass rejection filter (Kaiser Optical Systems Inc.) to minimize Rayleigh scatter into the monochromator. The Raman scattered light are collected with a 50 micron optical fiber. Using the “Raman Spectral Imaging” mode of the instrument, spectral images are obtained by scanning the sample in the x, z direction with a piezo driven xyz scan stage and collecting a spectrum at every pixel. Typical integration times are 0.3 s per pixel. The spectral images are 4800 total spectra corresponding to a physical scan dimension of 40 by 20 microns. For presentation of the confocal Raman data, images are generated based on unique properties of the spectra (i.e. integration of a Raman band, band height intensity, or band width). The microscope stage is modified with a custom-built sample holder that positioned and rotated the stents around their primary axis. The x direction is defined as the direction running parallel to the length of the stent and the z direction refers to the direction penetrating through the coating from the air-coating to the coating-metal interface. Typical laser power is <10 mW on the sample stage. All experiments can be conducted with a plan achromat objective, 100×N_{A}=0.9 (Nikon).

Samples (n=5) comprising stents made of L605 (0.05-0.15% C, 1.00-2.00% Mn, maximum 0.040% Si, maximum 0.030% P, maximum 0.3% S, 19.00-21.00% Cr, 9.00-11.00% Ni, 14.00-16.00% W, 3.00% Fe, and Bal. Co) and having coatings as described herein and/or produced by methods described herein can be analyzed. For each sample, three locations are selected along the stent length. The three locations are located within one-third portions of the stents so that the entire length of the stent are represented in the data. The stent is then rotated 180 degrees around the circumference and an additional three locations are sampled along the length. In each case, the data is collected from the strut portion of the stent. Six random spatial locations are also profiled on coated coupon samples made of L605 and having coatings as described herein and/or produced by methods described herein. The Raman spectra of each individual component present in the coatings are also collected for comparison and reference. Using the instrument software, the average spectra from the spectral image data are calculated by selecting the spectral image pixels that are exclusive to each layer. The average spectra are then exported into GRAMS/AI v. 7.02 software (Thermo Galactic) and the appropriate Raman bands are fit to a Voigt function. The band areas and shift positions are recorded.

The pure component spectrum for each component of the coating (e.g. drug, polymer) are also collected at 532 and 785 nm excitation. The 785 nm excitation spectra are collected with a confocal Raman microscope (WITec Instruments Corp. Savoy, Ill.) equipped with a 785 nm diode laser, appropriate collection optics, and a back-illuminated thermoelectriaclly cooled 1024×128 pixel array CCD camera optimized for visible and infrared wavelengths (Andor Technology).

### X-Ray Photoelectron Spectroscopy (XPS)

XPS can be used to quantitatively determine elemental species and chemical bonding environments at the outer 5-10 nm of sample surface. The technique can be operated in spectroscopy or imaging mode. When combined with a sputtering source XPS can be utilized to give depth profiling chemical characterization. XPS (ESCA) and other analytical techniques such as described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

For example, in one test, a sample comprising a stent coated by methods described herein and/or a device as described herein is obtained. XPS analysis is performed on a sample using a Physical Electronics Quantum 2000 Scanning ESCA. The monochromatic Al Kα source is operated at 15 kV with a power of 4.5 W. The analysis is done at a 45° take off angle. Three measurements are taken along the length of each sample with the analysis area ˜20 microns in diameter. Low energy electron and Ar⁺ ion floods are used for charge compensation.

### Time of Flight Secondary Ion Mass Spectrometers (TOF-SIMS)

TOF-SIMS can be used to determine molecular species (drug and polymer) at the outer 1-2 nm of sample surface when operated under static conditions. The technique can be operated in spectroscopy or imaging mode at high spatial resolution. Additionally cross-sectioned samples can be analysed. When operated under dynamic experimental conditions, known in the art, depth profiling chemical characterization can be achieved.

For example, to analyze the uppermost surface only, static conditions (for example a ToF-SIMS IV (IonToF, Munster)) using a 25 Kv Bi++ primary ion source maintained below 1012 ions per cm2 is used. Where necessary a low energy electron flood gun (0.6 nA DC) is used to charge compensate insulating samples.

Cluster Secondary Ion Mass Spectrometry, may be employed for depth profiling as described Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference.

For example, a stent as described herein is obtained. The stent is prepared for SIMS analysis by cutting it longitudinally and opening it up with tweezers. The stent is then pressed into multiple layers of indium foil with the outer diameter facing outward.

TOF-SIMS depth profiling experiments are performed using an Ion-TOF IV instrument equipped with both Bi and SF5+ primary ion beam cluster sources. Sputter depth profiling is performed in the dual-beam mode, whilst preserving the chemical integrity of the sample. The analysis source is a pulsed, 25-keV bismuth cluster ion source, which bombarded the surface at an incident angle of 45° to the surface normal. The target current is maintained at ˜0.3 pÅ (+10%) pulsed current with a raster size of 200 um×200 um for all experiments. Both positive and negative secondary ions are extracted from the sample into a reflectron-type time-of-flight mass spectrometer. The secondary ions are then detected by a microchannel plate detector with a post-acceleration energy of 10 kV. A low-energy electron flood gun is utilized for charge neutralization in the analysis mode.

The sputter source used is a 5-keV SF5+ cluster source also operated at an incident angle of 45° to the surface normal. For thin model samples on Si, the SF5+ current is maintained at ˜2.7 nÅ with a 750 um×750 um raster. For the thick samples on coupons and for the samples on stents, the current is maintained at 6 nA with a 500 um×500 um raster. All primary beam currents are measured with a Faraday cup both prior to and after depth profiling.

All depth profiles are acquired in the noninterlaced mode with a 5-ms pause between sputtering and analysis. Each spectrum is averaged over a 7.37 second time period. The analysis is immediately followed by 15 seconds of SF₅⁺ sputtering. For depth profiles of the surface and subsurface regions only, the sputtering time was decreased to 1 second for the 5% active agent sample and 2 seconds for both the 25% and 50% active agent samples.

Temperature-controlled depth profiles are obtained using a variable-temperature stage with Eurotherm Controls temperature controller and IPSG V3.08 software. samples are first placed into the analysis chamber at room temperature. The samples are brought to the desired temperature under ultra high-vacuum conditions and are allowed to stabilize for 1 minute prior to analysis. All depth profiling experiments are performed at −100 C and 25 C.

### Atomic Force Microscopy (AFM)

AFM is a high resolution surface characterization technique. AFM is used in the art to provide topographical imaging, in addition when employed in Tapping Mode™ can image material and or chemical properties of the surface. Additionally cross-sectioned samples can be analyzed. The technique can be used under ambient, solution, humidified or temperature controlled conditions. Other modes of operation are well known and can be readily employed here by those skilled in the art.

A stent as described herein is obtained. AFM is used to determine the structure of the drug polymer layers. AFM may be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

Polymer and drug morphologies, coating composition, at least may be determined using atomic force microscopy (AFM) analysis. A multi-mode AFM (Digital Instruments/Veeco Metrology, Santa Barbara, Calif.) controlled with Nanoscope Ma and NanoScope Extender electronics is used. Samples are examined in the dry state using AFM before elution of the drug (e.g. rapamycin). Samples are also examined at select time points through a elution period (e.g. 48 hours) by using an AFM probe-tip and flow-through stage built to permit analysis of wet samples. The wet samples are examined in the presence of the same elution medium used for in-vitro kinetic drug release analysis (e.g. PBS-Tween20, or 10 mM Tris, 0.4 wt. % SDS, pH 7.4). Saturation of the solution is prevented by frequent exchanges of the release medium with several volumes of fresh medium. TappingMode™ AFM imaging may be used to show topography (a real-space projection of the coating surface microstructure) and phase-angle changes of the AFM over the sample area to contrast differences in the materials properties. The AFM topography images can be three-dimensionally rendered to show the surface of a coated stent, which can show holes or voids of the coating which may occur as the polymer is absorbed and the drug is eluted over time, for example.

Scanning Electron Microscopy (SEM) with Focused Ion Beam (FIB) Milling

Stents as described herein, and or produced by methods described herein are visualized using SEM-FIB. Alternatively, a coated coupon could be tested in this method. Focused ion beam FIB is a tool that allows precise site-specific sectioning, milling and depositing of materials. FIB can be used in conjunction with SEM, at ambient or cryo conditions, to produce in-situ sectioning followed by high-resolution imaging. FIB-SEM can produce a cross-sectional image of the polymer and drug layers on the stent. The image can be used to quantitate the thickness of the layers and uniformity of the layer thickness at manufacture and at time points after stenting (or after in-vitro elution at various time points).

A FEI Dual Beam Strata 235 FIB/SEM system is a combination of a finely focused Ga ion beam (FIB) accelerated by 30 kV with a field emission electron beam in a scanning electron microscope instrument and is used for imaging and sectioning the stents. Both beams focus at the same point of the sample with a probe diameter less than 10 nm. The FIB can also produce thinned down sections for TEM analysis.

To prevent damaging the surface of the stent with incident ions, a Pt coating is first deposited via electron beam assisted deposition and ion beam deposition prior to FIB sectioning. For FIB sectioning, the Ga ion beam is accelerated to 30 kV and the sectioning process is about 2 h in duration. Completion of the FIB sectioning allows one to observe and quantify by SEM the thickness of the polymer layers that are, for example, left on the stent as they are absorbed.

### Example 5

### Analysis of the Thickness of a Device Coating

Analysis can be determined by either in-situ analysis or from cross-sectioned samples.

### X-Ray Photoelectron Spectroscopy (XPS)

XPS can be used to quantitatively determine the presence of elemental species and chemical bonding environments at the outer 5-10 nm of sample surface. The technique can be operated in spectroscopy or imaging mode. When combined with a sputtering source XPS can be utilized to give depth profiling chemical characterization. XPS (ESCA) and other analytical techniques such as described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

Thus, in one test, a sample comprising a stent coated by methods described herein and/or a device as described herein is obtained. XPS analysis is done on a sample using a Physical Electronics Quantum 2000 Scanning ESCA. The monochromatic Al Kα source is operated at 15 kV with a power of 4.5 W. The analysis is done at a 45° take off angle. Three measurements are taken along the length of each sample with the analysis area ˜20 microns in diameter. Low energy electron and Ar⁺ ion floods are used for charge compensation.

### Time of Flight Secondary Ion Mass Spectrometery

TOF-SIMS can be used to determine molecular species (drug and polymer) at the outer 1-2 nm of sample surface when operated under static conditions. The technique can be operated in spectroscopy or imaging mode at high spatial resolution. Additionally cross-sectioned samples can be analysed. When operated under dynamic experimental conditions, known in the art, depth profiling chemical characterization can be achieved.

For example, under static conditions (for example a ToF-SIMS IV (IonToF, Munster)) using a 25 Kv Bi⁺⁺ primary ion source maintained below 10¹² ions per cm² is used. Where necessary a low energy electron flood gun (0.6 nA DC) is used to charge compensate insulating samples.

Cluster Secondary Ion Mass Spectrometry, may be employed for depth profiling as described Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference.

A stent as described herein is obtained. The stent is prepared for SIMS analysis by cutting it longitudinally and opening it up with tweezers. The stent is then pressed into multiple layers of iridium foil with the outer diameter facing outward.

TOF-SIMS experiments are performed on an Ion-TOF IV instrument equipped with both Bi and SF5+ primary ion beam cluster sources. Sputter depth profiling is performed in the dual-beam mode. The analysis source is a pulsed, 25-keV bismuth cluster ion source, which bombarded the surface at an incident angle of 45° to the surface normal. The target current is maintained at ˜0.3 pÅ (+10%) pulsed current with a raster size of 200 um×200 um for all experiments. Both positive and negative secondary ions are extracted from the sample into a reflectron-type time-of-flight mass spectrometer. The secondary ions are then detected by a microchannel plate detector with a post-acceleration energy of 10 kV. A low-energy electron flood gun is utilized for charge neutralization in the analysis mode.

The sputter source used is a 5-keV SF5+ cluster source also operated at an incident angle of 45° to the surface normal. For thin model samples on Si, the SF5+ current is maintained at ˜2.7 nÅ with a 750 um×750 um raster. For the thick samples on coupons and for the samples on stents, the current is maintained at 6 nA with a 500 um×500 um raster. All primary beam currents are measured with a Faraday cup both prior to and after depth profiling.

All depth profiles are acquired in the noninterlaced mode with a 5-ms pause between sputtering and analysis. Each spectrum is averaged over a 7.37 second time period. The analysis is immediately followed by 15 seconds of SF₅⁺ sputtering. For depth profiles of the surface and subsurface regions only, the sputtering time was decreased to 1 second for the 5% active agent sample and 2 seconds for both the 25% and 50% active agent samples. Temperature-controlled depth profiles are obtained using a variable-temperature stage with Eurotherm Controls temperature controller and IPSG V3.08 software. samples are first placed into the analysis chamber at room temperature. The samples are brought to the desired temperature under ultra high-vacuum conditions and are allowed to stabilize for 1 minute prior to analysis. All depth profiling experiments are performed at −100 C and 25 C.

### Atomic Force Microscopy (AFM)

AFM is a high resolution surface characterization technique. AFM is used in the art to provide topographical imaging, in addition when employed in Tapping Mode™ can image material and or chemical properties of the surface. Additionally cross-sectioned samples can be analyzed.

A stent as described herein is obtained. AFM may be alternatively be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

Polymer and drug morphologies, coating composition, and cross-sectional thickness at least may be determined using atomic force microscopy (AFM) analysis. A multi-mode AFM (Digital Instruments/Veeco Metrology, Santa Barbara, Calif.) controlled with Nanoscope Ma and NanoScope Extender electronics is usedTappingMode™ AFM imaging may be used to show topography (a real-space projection of the coating surface microstructure) and phase-angle changes of the AFM over the sample area to contrast differences in the materials properties. The AFM topography images can be three-dimensionally rendered to show the surface of a coated stent or cross-section. Scanning Electron Microscopy (SEM) with Focused Ion Beam (FIB) Stents as described herein, and or produced by methods described herein are visualized using SEM-FIB analysis. Alternatively, a coated coupon could be tested in this method. Focused ion beam FIB is a tool that allows precise site-specific sectioning, milling and depositing of materials. FIB can be used in conjunction with SEM, at ambient or cryo conditions, to produce in-situ sectioning followed by high-resolution imaging. FIB-SEM can produce a cross-sectional image of the polymer layers on the stent. The image can be used to quantitate the thickness of the layers as well as show whether there is uniformity of the layer thickness at manufacture and at time points after stenting (or after in-vitro elution at various time points).

A FEI Dual Beam Strata 235 FIB/SEM system is a combination of a finely focused Ga ion beam (FIB) accelerated by 30 kV with a field emission electron beam in a scanning electron microscope instrument and is used for imaging and sectioning the stents. Both beams focus at the same point of the sample with a probe diameter less than 10 nm. The FIB can also produce thinned down sections for TEM analysis.

To prevent damaging the surface of the stent with incident ions, a Pt coating is first deposited via electron beam assisted deposition and ion beam deposition prior to FIB sectioning. For FIB sectioning, the Ga ion beam is accelerated to 30 kV and the sectioning process is about 2 h in duration. Completion of the FIB sectioning allows one to observe and quantify by SEM the thickness of the polymer layers that are, for example, left on the stent as they are absorbed.

### Interferometry

Interferometry may additionally and/or alternatively used to determine the thickness of the coating as noted in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

### Ellipsometry

Ellipsometry is sensitive measurement technique for coating analysis on a coupon. It uses polarized light to probe the dielectric properties of a sample. Through an analysis of the state of polarization of the light that is reflected from the sample the technique allows the accurate characterization of the layer thickness and uniformity. Thickness determinations ranging from a few angstroms to tens of microns are possible for single layers or multilayer systems. See, for example, Jewell, et al., “Release of Plasmid DNA from Intravascular Stents Coated with Ultrathin Mulyikayered Polyelectrolyte Films” Biomacromolecules. 7: 2483-2491 (2006) incorporated herein in its entirety by reference.

### Example 6

### Analysis of the Thickness of a Device

### Scanning Electron Microscopy (SEM)

A sample coated stent described herein is obtained. Thickness of the device can be assessed using this analytical technique. The thickness of multiple struts were taken to ensure reproducibility and to characterize the coating and stent. The thickness of the coating was observed by SEM using a Hitachi S-4800 with an accelerating voltage of 800V. Various magnifications are used. SEM can provide top-down and cross-section images at various magnifications.

### Nano X-Ray Computer Tomography

Another technique that may be used to view the physical structure of a device in 3-D is Nano X-Ray Computer Tomography (e.g. such as made by SkyScan).

### Example 7

### Determination of the Type or Composition of a Polymer Coating a Device

### Nuclear Magnetic Resonance (NMR)

Composition of the polymer samples before and after elution can be determined by ¹H NMR spectrometry as described in Xu et al., “Biodegradation of poly(1-lactide-co-glycolide tube stents in bile” Polymer Degradation and Stability. 93:811-817 (2008) incorporated herein in its entirety by reference. Compositions of polymer samples are determined for example using a 300M Bruker spectrometer with d-chloroform as solvent at room temperature.

### Raman Spectroscopy

FT-Raman or confocal raman microscopy can be employed to determine composition.

For example, a sample (a coated stent) is prepared as described herein. Images are taken on the coating using Raman Spectroscopy. Alternatively, a coated coupon could be tested in this method. To test a sample using Raman microscopy and in particular confocal Raman microscopy, it is understood that to get appropriate Raman high resolution spectra sufficient acquisition time, laser power, laser wavelength, sample step size and microscope objective need to be optimized. Raman spectroscopy and other analytical techniques such as described in Balss, et al., “Quantitative spatial distribution of sirolimus and polymers in drug-eluting stents using confocal Raman microscopy” J. of Biomedical Materials Research Part A, 258-270 (2007), incorporated in its entirety herein by reference, and/or described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

For example a WITec CRM 200 scanning confocal Raman microscope using a Nd:YAG laser at 532 nm is applied in the Raman imaging mode. The sample is placed upon a piezoelectrically driven table, the laser light is focused upon the sample using a 100×dry objective (numerical aperture 0.90), and the finely focused laser spot is scanned into the sample. As the laser scans the sample, over each 0.33 micron interval a Raman spectrum with high signal to noise is collected using 0.3 Seconds of integration time. Each confocal cross-sectional image of the coatings displays a region 70 μm wide by 10 μm deep, and results from the gathering of 6300 spectra with a total imaging time of 32 min. Multivariate analysis using reference spectra from samples of rapamycin (amorphous and crystalline) and polymer references are used to deconvolve the spectral data sets, to provide chemical maps of the distribution.

In another test, spectral depth profiles of samples are performed with a CRM200 microscope system from WITec Instruments Corporation (Savoy, Ill.). The instrument is equipped with a NdYAG frequency doubled laser (532 excitation), a single monochromator (Acton) employing a 600 groove/mm grating and a thermoelectrically cooled 1024 by 128 pixel array CCD camera (Andor Technology). The microscope is equipeed with appropriate collection optics that include a holographic laser bandpass rejection filter (Kaiser Optical Systems Inc.) to minimize Rayleigh scatter into the monochromator. The Raman scattered light are collected with a 50 micron optical fiber. Using the “Raman Spectral Imaging” mode of the instrument, spectral images are obtained by scanning the sample in the x, z direction with a piezo driven xyz scan stage and collecting a spectrum at every pixel. Typical integration times are 0.3 s per pixel. The spectral images are 4800 total spectra corresponding to a physical scan dimension of 40 by 20 microns. For presentation of the confocal Raman data, images are generated base don unique properties of the spectra (i.e. integration of a Raman band, band height intensity, or band width). The microscope stage is modified with a custom-built sample holder that positioned and rotated the stents around their primary axis. The x direction is defined as the direction running parallel to the length of the stent and the z direction refers to the direction penetrating through the coating from the air-coating to the coating-metal interface. Typical laser power is <10 mW on the sample stage. All experiments can be conducted with a plan achromat objective, 100×N_{A}=0.9 (Nikon).

Samples (n=5) comprising stents made of L605 and having coatings as described herein and/or produced by methods described herein can be analyzed. For each sample, three locations are selected along the stent length. The three locations are located within one-third portions of the stents so that the entire length of the stent are represented in the data. The stent is then rotated 180 degrees around the circumference and an additional three locations are sampled along the length. In each case, the data is collected from the strut portion of the stetn. Six random spatial locations are also profiled on coated coupon samples made of L605 and having coatings as described herein and/or produced by methods described herein. The Raman spectra of each individual component present in the coatings are also collected for comparison and reference. Using the instrument software, the average spectra from the spectral image data are calculated by selecting the spectral image pixels that are exclusive to each layer. The average spectra are then exported into GRAMS/AI v. 7.02 software (Thermo Galactic) and the appropriate Raman bands are fit to a Voigt function. The band areas and shift positions are recorded.

The pure component spectrum for each component of the coating (e.g. drug, polymer) are also collected at 532 and 785 nm excitation. The 785 nm excitation spectra are collected with a confocal Raman microscope (WITec Instruments Corp. Savoy, Ill.) equipped with a 785 nm diode laser, appropriate collection optics, and a back-illuminated thermoelectriaclly cooled 1024×128 pixel array CCD camera optimized for visible and infrared wavelengths (Andor Technology).

### Time of Flight Secondary Ion Mass Spectrometers

TOF-SIMS can be used to determine molecular species (drug and polymer) at the outer 1-2 nm of sample surface when operated under static conditions. The technique can be operated in spectroscopy or imaging mode at high spatial resolution. Additionally cross-sectioned samples can be analysed. When operated under dynamic experimental conditions, known in the art, depth profiling chemical characterization can be achieved.

For example, under static conditions (for example a ToF-SIMS IV (IonToF, Munster)) using a 25 Kv Bi⁺⁺ primary ion source maintained below 10¹² ions per cm² is used. Where necessary a low energy electron flood gun (0.6 nA DC) is used to charge compensate insulating samples.

Cluster Secondary Ion Mass Spectrometry, may be employed as described Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference.

A stent as described herein is obtained. The stent is prepared for SIMS analysis by cutting it longitudinally and opening it up with tweezers. The stent is then pressed into multiple layers of iridium foil with the outer diameter facing outward.

TOF-SIMS experiments are performed on an Ion-TOF IV instrument equipped with both B1 and SF5+ primary ion beam cluster sources. Sputter depth profiling is performed in the dual-beam mode. The analysis source is a pulsed, 25-keV bismuth cluster ion source, which bombarded the surface at an incident angle of 45° to the surface normal. The target current is maintained at ˜0.3 pÅ (+10%) pulsed current with a raster size of 200 um×200 um for all experiments. Both positive and negative secondary ions are extracted from the sample into a reflectron-type time-of-flight mass spectrometer. The secondary ions are then detected by a microchannel plate detector with a post-acceleration energy of 10 kV. A low-energy electron flood gun is utilized for charge neutralization in the analysis mode.

The sputter source used is a 5-keV SF5+ cluster source also operated at an incident angle of 45° to the surface normal. For thin model samples on Si, the SF5+current is maintained at ˜2.7 nÅ with a 750 um×750 um raster. For the thick samples on coupons and for the samples on stents, the current is maintained at 6 nA with a 500 um×500 um raster. All primary beam currents are measured with a Faraday cup both prior to and after depth profiling.

All depth profiles are acquired in the noninterlaced mode with a 5-ms pause between sputtering and analysis. Each spectrum is averaged over a 7.37 second time period. The analysis is immediately followed by 15 seconds of SF₅⁺ sputtering. For depth profiles of the surface and subsurface regions only, the sputtering time was decreased to 1 second for the 5% active agent sample and 2 seconds for both the 25% and 50% active agent samples.

Temperature-controlled depth profiles are obtained using a variable-temperature stage with Eurotherm Controls temperature controller and IPSG V3.08 software. Samples are first placed into the analysis chamber at room temperature. The samples are brought to the desired temperature under ultra high-vacuum conditions and are allowed to stabilize for 1 minute prior to analysis. All depth profiling experiments are performed at −100 C and 25 C.

### Atomic Force Microscopy (AFM)

AFM is a high resolution surface characterization technique. AFM is used in the art to provide topographical imaging, in addition when employed in Tapping Mode™ can image material and or chemical properties of the surface. Additionally cross-sectioned samples can be analyzed. Coating composition may be determined using Tapping Mode™ atomic force microscopy (AFM) analysis. Other modes of operation are well known and can be employed here by those skilled in the art.

A stent as described herein is obtained. AFM may be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

Polymer and drug morphologies, coating composition, at least may be determined using atomic force microscopy (AFM) analysis. A multi-mode AFM (Digital Instruments/Veeco Metrology, Santa Barbara, Calif.) controlled with Nanoscope Ma and NanoScope Extender electronics is used. TappingMode™ AFM imaging may be used to show topography (a real-space projection of the coating surface microstructure) and phase-angle changes of the AFM over the sample area to contrast differences in the materials properties.

### Infrared (IR) Spectroscopy for In-Vitro Testing

Infrared (IR) Spectroscopy using FTIR, ATR-IR or micro ATR-IR can be used to identify polymer composition by comparison to standard polymer reference spectra.

### Example 8

### Determination of the Bioabsorbability of a Device

In some embodiments of the device the substrate coated itself is made of a bioabsorbable material, such as the bioabsorbable polymers presented herein, or another bioabsorbable material such as magnesium and, thus, the entire device is bioabsorbable. Techniques presented with respect to showing Bioabsorbability of a polymer coating may be used to additionally and/or alternatively show the bioabsorbability of a device, for example, by GPC In-Vivo testing, HPLC In-Vivo Testing, GPC In-Vitro testing, HPLC In-Vitro Testing, SEM-FIB Testing, Raman Spectroscopy, SEM, and XPS as described herein with variations and adjustments which would be obvious to those skilled in the art. Another technique to view the physical structure of a device in 3-D is Nano X-Ray Computer Tomography (e.g. such as made by SkyScan), which could be used in an elution test and/or bioabsorbability test, as described herein to show the physical structure of the coating remaining on stents at each time point, as compared to a scan prior to elution/bioabsorbtion.

### Example 9

### Determination of Secondary Structures Presence of a Biological Agent

### Raman Spectroscopy

FT—Raman or confocal raman microscopy can be employed to determine secondary structure of a biological Agent. For example fitting of the Amide I, II, or III regions of the Raman spectrum can elucidate secondary structures (e.g. alpha-helices, beta-sheets). See, for example, Iconomidou, et al., “Secondary Structure of Chorion Proteins of the Teleosetan Fish Dentex dentex by ATR FR-IR and FT-Raman Spectroscopy” J. of Structural Biology, 132, 112-122 (2000); Griebenow, et al., “On Protein Denaturation in Aqueous-Organic Mixtures but Not in Pure Organic Solvents” J. Am. Chem. Soc., Vol 118, No. 47, 11695-11700 (1996).

### Infrared (IR) Spectroscopy for In-Vitro Testing

Infrared spectroscopy, for example FTIR, ATR-IR and micro ATR-IR can be employed to determine secondary structure of a biological Agent. For example fitting of the Amide I, II, of III regions of the infrared spectrum can elucidate secondary structures (e.g. alpha-helices, beta-sheets).

### Example 10

### Determination of the Microstructure of a Coating on a Medical Device

### Atomic Force Microscopy (AFM)

AFM is a high resolution surface characterization technique. AFM is used in the art to provide topographical imaging, in addition when employed in Tapping Mode™ can image material and or chemical properties of the surface. Additionally cross-sectioned samples can be analyzed. The technique can be used under ambient, solution, humidified or temperature controlled conditions. Other modes of operation are well known and can be readily employed here by those skilled in the art.

A stent as described herein is obtained. AFM is used to determine the microstructure of the coating. A stent as described herein is obtained. AFM may be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

For example, polymer and drug morphologies, coating composition, and physical structure may be determined using atomic force microscopy (AFM) analysis. A multi-mode AFM (Digital Instruments/Veeco Metrology, Santa Barbara, Calif.) controlled with Nanoscope IIIa and NanoScope Extender electronics is used. Samples are examined in the dry state using AFM before elution of the drug (e.g. rapamycin). Samples are also examined at select time points through a elution period (e.g. 48 hours) by using an AFM probe-tip and flow-through stage built to permit analysis of wet samples. The wet samples are examined in the presence of the same elution medium used for in-vitro kinetic drug release analysis (e.g. PBS-Tween20, or 10 mM Tris, 0.4 wt. % SDS, pH 7.4). Saturation of the solution is prevented by frequent exchanges of the release medium with several volumes of fresh medium. TappingMode™ AFM imaging may be used to show topography (a real-space projection of the coating surface microstructure) and phase-angle changes of the AFM over the sample area to contrast differences in the materials properties. The AFM topography images can be three-dimensionally rendered to show the surface of a coated stent, which can show holes or voids of the coating which may occur as the polymer is absorbed and the drug is released from the polymer over time, for example.

### Nano X-Ray Computer Tomography

Another technique that may be used to view the physical structure of a device in 3-D is Nano X-Ray Computer Tomography (e.g. such as made by SkyScan), which could be used in an elution test and/or bioabsorbability test, as described herein to show the physical structure of the coating remaining on stents at each time point, as compared to a scan prior to elution/bioabsorbtion.

### Example 11

### Determination of an Elution Profile

### In Vitro

### Example 11a

In one method, a stent described herein is obtained. The elution profile is determined as follows: stents are placed in 16 mL test tubes and 15 mL of 10 mM PBS (pH 7.4) is pipetted on top. The tubes are capped and incubated at 37 C with end-over-end rotation at 8 rpm. Solutions are then collected at the designated time points (e.g. 1 d, 7 d, 14 d, 21 d, and 28 d) (e.g. 1 week, 2 weeks, and 10 weeks) and replenished with fresh 1.5 ml solutions at each time point to prevent saturation. One mL of DCM is added to the collected sample of buffer and the tubes are capped and shaken for one minute and then centrifuged at 200×G for 2 minutes. The supernatant is discarded and the DCM phase is evaporated to dryness under gentle heat (40° C.) and nitrogen gas. The dried DCM is reconstituted in 1 mL of 60:40 acetonitrile:water (v/v) and analyzed by HPLC. HPLC analysis is performed using Waters HPLC system (mobile phase 58:37:5 acetonitrile:water:methanol 1 mL/min, 20 uL injection, C18 Novapak Waters column with detection at 232 nm).

### Example 11b

In another method, the in vitro pharmaceutical agent elution profile is determined by a procedure comprising contacting the device with an elution media comprising ethanol (5%) wherein the pH of the media is about 7.4 and wherein the device is contacted with the elution media at a temperature of about 37° C. The elution media containing the device is optionally agitating the elution media during the contacting step. The device is removed (and/or the elution media is removed) at least at designated time points (e.g. 1 h, 3 h, 5 h, 7 h, 1 d, and daily up to 28 d) (e.g. 1 week, 2 weeks, and 10 weeks). The elution media is then assayed using a UV-Vis for determination of the pharmaceutical agent content. The elution media is replaced at each time point with fresh elution media to avoid saturation of the elution media. Calibration standards containing known amounts of drug were also held in elution media for the same durations as the samples and used at each time point to determine the amount of drug eluted at that time (in absolute amount and as a cumulative amount eluted).

In one test, devices were coated tested using this method. In these experiments (also reference Example 1) two different polymers were employed: Polymer A:—50:50 PLGA-Ester End Group, MW˜19 kD, degradation rate ˜70 days; Polymer B:—50:50 PLGA-Carboxylate End Group, MW˜10 kD, degradation rate ˜28 days. Metal stents were coated as follows: AS1: (n=6) Polymer A/Rapamycin/Polymer A/Rapamycin/Polymer A; AS2: (n=6) Polymer A/Rapamycin/Polymer A/Rapamycin/Polymer B; AS1 (213) also called AS1(B) elsewhere herein: (n=6) Polymer B/Rapamycin/Polymer B/Rapamycin/Polymer B; AS1b: (n=6) Polymer A/Rapamycin/Polymer A/Rapamycin/Polymer A; AS2b: (n=6) Polymer A/Rapamycin/Polymer A/Rapamycin/Polymer B. The in vitro pharmaceutical agent elution profile was determined by contacting each device with an elution media comprising ethanol (5%) wherein the pH of the media is about 7.4 and wherein the device was contacted with the elution media at a temperature of about 37° C. The elution media was removed from device contact at least at 1 h, 3 h, 5 h, 7 h, 1 d, and at additional time points up to 70 days (See FIGS. 1-4). The elution media was then assayed using a UV-Vis for determination of the pharmaceutical agent content (in absolute amount and cumulative amount eluted). The elution media was replaced at each time point with fresh elution media to avoid saturation of the elution media. Calibration standards containing known amounts of drug were also held in elution media for the same durations as the samples and assayed by UV-Vis at each time point to determine the amount of drug eluted at that time (in absolute amount and as a cumulative amount eluted), compared to a blank comprising Spectroscopic grade ethanol. Elution profiles as shown in FIGS. 1-4, showing the average amount of rapamycin eluted at each time point (average of all stents tested) in micrograms. Table 2 shows for each set of stents (n=6) in each group (AS1, AS2, AS(213), AS1b, AS2b), the average amount of rapamycin in ug loaded on the stents, the average amount of polymer in ug loaded on the stents, and the total amount of rapamycin and polymer in ug loaded on the stents.

TABLE 2

| | | | Ave. |
|---|---|---|---|
| Stent | Ave. | Ave. | Total |
| Coating | Rapa, ug | Poly, ug | Mass, ug |
| AS1 | 175 | 603 | 778 |
| AS2 | 153 | 717 | 870 |
| AS1(213) | 224 | 737 | 961 |
| AS1b | 171 | 322 | 493 |
| AS2b | 167 | 380 | 547 |

### Example 11c

In another method, the in vitro pharmaceutical agent elution profile is determined by a procedure comprising contacting the device with an elution media comprising ethanol (20%) and phosphate buffered saline (80%) wherein the pH of the media is about 7.4 and wherein the device is contacted with the elution media at a temperature of about 37° C. The elution media containing the device is optionally agitating the elution media during the contacting step. The device is removed (and/or the elution media is removed) at least at designated time points (e.g. 1 h, 3 h, 5 h, 7 h, 1 d, and daily up to 28 d) (e.g. 1 week, 2 weeks, and 10 weeks). The elution media is replaced periodically (at least at each time point, and/or daily between later time points) to prevent saturation; the collected media are pooled together for each time point. The elution media is then assayed for determination of the pharmaceutical agent content using HPLC. The elution media is replaced at each time point with fresh elution media to avoid saturation of the elution media. Calibration standards containing known amounts of drug are also held in elution media for the same durations as the samples and used at each time point to determine the amount of drug eluted at that time (in absolute amount and as a cumulative amount eluted). Where the elution method changes the drug over time, resulting in multiple peaks present for the drug when tested, the use of these calibration standards will also show this change, and allows for adding all the peaks to give the amount of drug eluted at that time period (in absolute amount and as a cumulative amount eluted).

In one test, devices (n=9, laminate coated stents) as described herein were coated and tested using this method. In these experiments a single polymer was employed: Polymer A: 50:50 PLGA-Ester End Group, MW˜19 kD. The metal (stainless steel) stents were coated as follows: Polymer A/Rapamycin/Polymer A/Rapamycin/Polymer A, and the average amount of rapamycin on each stent was 162 ug (stdev 27 ug). The coated stents were contacted with an elution media (5.00 mL) comprising ethanol (20%) and phosphate buffered saline wherein the pH of the media is about 7.4 (adjusted with potassiume carbonate solution—1 g/100 mL distilled water) and wherein the device is contacted with the elution media at a temperature of about 37° C.+/−0.2° C. The elution media containing the device was agitated in the elution media during the contacting step. The elution media was removed at least at time points of 1 h, 3 h, 5 h, 7 h, 1 d, and daily up to 28 d. The elution media was assayed for determination of the pharmaceutical agent (rapamycin) content using HPLC. The elution media was replaced at each time point with fresh elution media to avoid saturation of the elution media. Calibration standards containing known amounts of drug were also held in elution media for the same durations as the samples and assayed at each time point to determine the amount of drug eluted at that time (in absolute amount and as a cumulative amount eluted). The multiple peaks present for the rapamycin (also present in the calibration standards) were added to give the amount of drug eluted at that time period (in absolute amount and as a cumulative amount eluted). HPLC analysis is performed using Waters HPLC system, set up and run on each sample as provided in the Table 3 below using an injection volume of 100 uL.

TABLE 3

| Time point | | % Ammonium Acetate | Flow Rate |
|---|---|---|---|
| (minutes) | % Acetonitrile | (0.5%), pH 7.4 | (mL/min) |
| 0.00 | 10 | 90 | 1.2 |
| 1.00 | 10 | 90 | 1.2 |
| 12.5 | 95 | 5 | 1.2 |
| 13.5 | 100 | 0 | 1.2 |
| 14.0 | 100 | 0 | 3 |
| 16.0 | 100 | 0 | 3 |
| 17.0 | 10 | 90 | 2 |
| 20.0 | 10 | 90 | 0 |

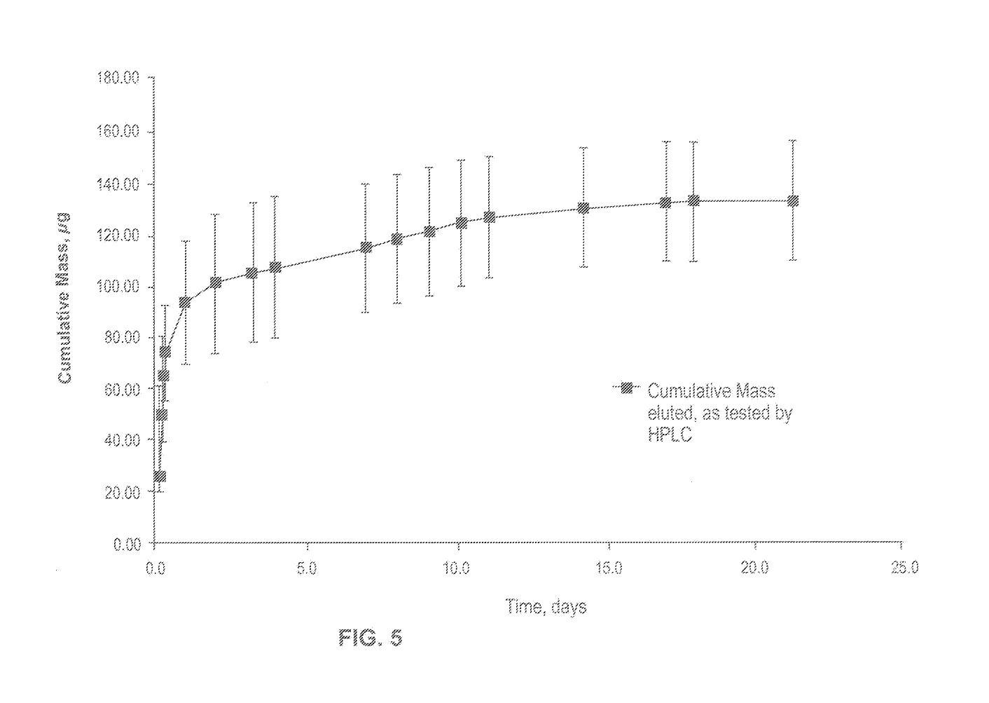

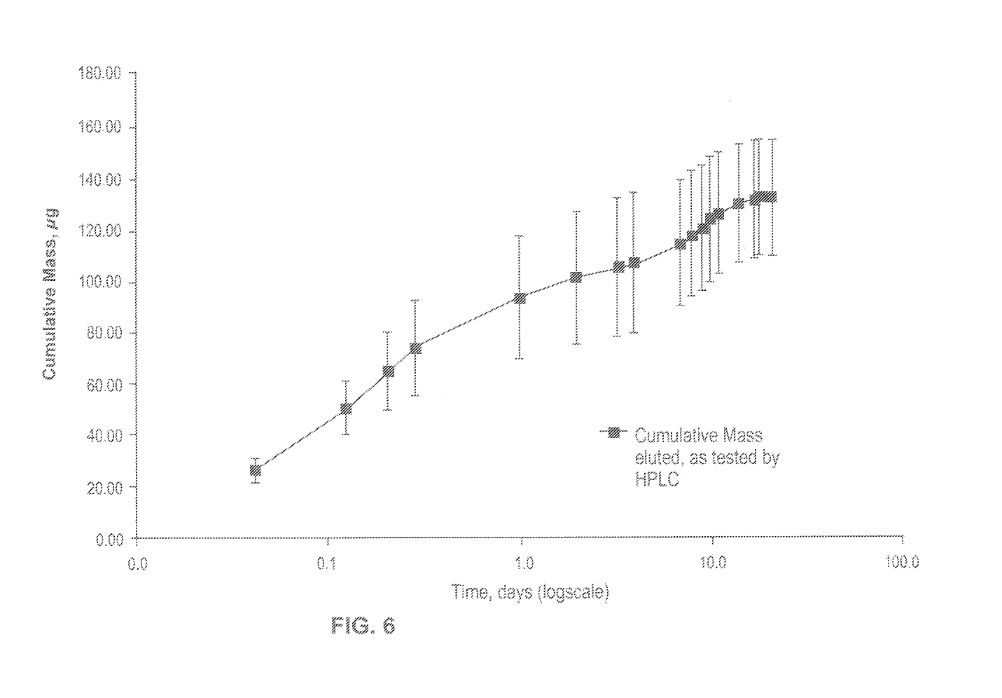

FIG. 5 elution profiles resulted, showing the average cumulative amount of rapamycin eluted at each time point (average of n=9 stents tested) in micrograms. FIG. 6 also expresses the same elution profile, graphed on a logarithmic scale (x-axis is log(time)).

### Example 11d

To obtain an accelerated in-vitro elution profile, an accelerated elution buffer comprising 18% v/v of a stock solution of 0.067 mol/L KH2PO4 and 82% v/v of a stock solution of 0.067 mol/L Na2HPO4 with a pH of 7.4 is used. Stents described herein are expanded and then placed in 1.5 ml solution of this accelerated elution in a 70° C. bath with rotation at 70 rpm. The solutions are then collected at the following time points: 0 min., 15 min., 30 min., 1 hr, 2 hr, 4 hr, 6 hr, 8 hr, 12 hr, 16 hr, 20 hr, 24 hr, 30 hr, 36 hr and 48 hr. Fresh accelerated elution buffer are added periodically at least at each time point to replace the incubated buffers that are collected and saved in order to prevent saturation. For time points where multiple elution media are used (refreshed between time points), the multiple collected solutions are pooled together for liquid extraction by dichloromethane. Dichloromethane extraction and HPLC analysis is performed in the manner described previously.

### In Vivo

### Example 11e

Rabbit in vivo models as described above are euthanized at multiple time points. Stents are explanted from the rabbits. The explanted stents are placed in 16 mL test tubes and 15 mL of 10 mM PBS (pH 7.4) is pipette on top. One mL of DCM is added to the buffer and the tubes are capped and shaken for one minute and then centrifuged at 200×G for 2 minutes. The supernatant is discarded and the DCM phase is evaporated to dryness under gentle heat (40° C.) and nitrogen gas. The dried DCM is reconstituted in 1 mL of 60:40 acetonitrile:water (v/v) and analyzed by HPLC. HPLC analysis is performed using Waters HPLC system (mobile phase 58:37:5 acetonitrile:water:methanol 1 mL/min, 20 uL injection, C18 Novapak Waters column with detection at 232 nm).

### Example 12

### Determination of the Conformability (Conformality) of a Device Coating, and/or Determiniation of Device or Substrate Breakage and Coating Penetration Thereby

The ability to uniformly coat stents with controlled composition and thickness using electrostatic capture in a rapid expansion of supercritical solution (RESS) experimental series has been demonstrated.

### Scanning Electron Microscopy (SEM)

Stents are observed by SEM using a Hitachi S-4800 with an accelerating voltage of 800V. Various magnifications are used to evaluate the integrity, especially at high strain regions. SEM can provide top-down and cross-section images at various magnifications. Coating uniformity and thickness can also be assessed using this analytical technique. Various magnifications are used to evaluate the integrity, especially at high strain regions of the substrate and or device generally. SEM can provide top-down and cross-section images at various magnifications to determine if a broken piece of the device and/or substrate penetrated the coating.

Pre- and post-expansions stents are observed by SEM using a Hitachi S-4800 with an accelerating voltage of 800V. Various magnifications are used to evaluate the integrity of the layers, especially at high strain regions and or of the substrate or device integrity (to detect broken substrate piece or device piece and/or penetration of the cotaing by such broken piece(s)).

Scanning Electron Microscopy (SEM) with Focused Ion Beam (FIB)

Stents as described herein, and or produced by methods described herein are visualized using SEM-FIB analysis. Alternatively, a coated coupon could be tested in this method. Focused ion beam FIB is a tool that allows precise site-specific sectioning, milling and depositing of materials. FIB can be used in conjunction with SEM, at ambient or cryo conditions, to produce in-situ sectioning followed by high-resolution imaging. Cross-sectional FIB images may be acquired, for example, at 7000× and/or at 20000× magnification. An even coating of consistent thickness is visible. A device that has a broken piece may be imaged using this method to determine whether the broken piece penetrated the coating.

### Optical Microscopy

An Optical micrscope may be used to create and inspect the stents and to empirically survey the coating of the substrate (e.g. coating uniformity). Nanoparticles of the drug and/or the polymer can be seen on the surfaces of the substrate using this analytical method. Following sintering, the coatings can be see using this method to view the coating conformaliy and for evidence of crystallinity of the drug. The device may thus be evaluated for broken substrate piece or broken device piece and to determine whether such broken substrate penetrated the coating.

### Example 13

### Determination of the Total Content of the Active Agent

Determination of the total content of the active agent in a coated stent may be tested using techniques described herein as well as other techniques obvious to one of skill in the art, for example using GPC and HPLC techniques to extract the drug from the coated stent and determine the total content of drug in the sample.

UV-VIS can be used to quantitatively determine the mass of rapamycin coated onto the stents. A UV-Vis spectrum of Rapamycin can be shown and a Rapamycin calibration curve can be obtained, (e.g. λ@ 277 nm in ethanol). Rapamycin is then dissolved from the coated stent in ethanol, and the drug concentration and mass calculated.

In one test, the total amount of rapamycin present in units of micrograms per stent is determined by reverse phase high performance liquid chromatography with UV detection (RP-HPLC-UV). The analysis is performed with modifications of literature-based HPLC methods for rapamycin that would be obvious to a person of skill in the art. The average drug content of samples (n=10) from devices comprising stents and coatings as described herein, and/or methods described herein are tested.

### Example 14

### Determination of the Extent of Aggregation of an Active Agent

### Raman Spectroscopy

Confocal Raman microscopy can be used to characterize the drug aggregation by mapping in the x-y or x-z direction. Additionally cross-sectioned samples can be analysed. Raman spectroscopy and other analytical techniques such as described in Balss, et al., “Quantitative spatial distribution of sirolimus and polymers in drug-eluting stents using confocal Raman microscopy” J. of Biomedical Materials Research Part A, 258-270 (2007), incorporated in its entirety herein by reference, and/or described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference may be used.

A sample (a coated stent) is prepared as described herein. Images are taken on the coating using Raman Spectroscopy. Alternatively, a coated coupon could be tested in this method. A WITec CRM 200 scanning confocal Raman microscope using a NiYAG laser at 532 nm is applied in the Raman imaging mode. The sample is place upon a piezoelectrically driven table, the laser light is focused upon the sample using a 100×dry objective (numerical aperture 0.90), and the finely focused laser spot is scanned into the sample. As the laser scans the sample, over each 0.33 micron interval a Raman spectrum with high signal to noise is collected using 0.3 Seconds of integration time. Each confocal cross-sectional image of the coatings displays a region 70 μm wide by 10 μm deep, and results from the gathering of 6300 spectra with a total imaging time of 32 min. To deconvolute the spectra and obtain separate images of the active agent and the polymer, all the spectral data (6300 spectra over the entire spectral region 500-3500 cm⁻¹) are processed using an augmented classical least squares algorithm (Eigenvector Research, Wenatchee Wash.) using basis spectra obtained from samples of rapamycin (amorphous and crystalline) and polymer. For each sample, several areas are measured by Raman to ensure that results are reproducible, and to show layering of drug and polymer through the coating. Confocal Raman Spectroscopy can profile down micron by micron, can show the composition of the coating through the thickness of the coating.

### Time of Flight Secondary Ion Mass Spectrometers

TOF-SIMS can be used to determine drug aggregation at the outer 1-2 nm of sample surface when operated under static conditions. The technique can be operated in spectroscopy or imaging mode at high spatial resolution. Additionally cross-sectioned samples can be analysed. When operated under dynamic experimental conditions, known in the art, depth profiling chemical characterization can be achieved.

For example, under static conditions (for example a ToF-SIMS IV (IonToF, Munster)) using a 25 Kv Bi++ primary ion source maintained below 1012 ions per cm2 is used. Where necessary a low energy electron flood gun (0.6 nA DC) is used to charge compensate insulating samples.

Cluster Secondary Ion Mass Spectrometry, may be employed as described in Belu et al., “Three-Dimensional Compositional Analysis of Drug Eluting Stent Coatings Using Cluster Secondary Ion Mass Spectroscopy” Anal. Chem. 80: 624-632 (2008) incorporated herein in its entirety by reference.

A stent as described herein is obtained. The stent is prepared for SIMS analysis by cutting it longitudinally and opening it up with tweezers. The stent is then pressed into multiple layers of iridium foil with the outer diameter facing outward.

For example TOF-SIMS experiments are performed on an Ion-TOF IV instrument equipped with both Bi and SF5+ primary ion beam cluster sources. Sputter depth profiling is performed in the dual-beam mode. The analysis source is a pulsed, 25-keV bismuth cluster ion source, which bombarded the surface at an incident angle of 45° to the surface normal. The target current is maintained at ˜0.3 pÅ (+10%) pulsed current with a raster size of 200 um×200 um for all experiments. Both positive and negative secondary ions are extracted from the sample into a reflectron-type time-of-flight mass spectrometer. The secondary ions are then detected by a microchannel plate detector with a post-acceleration energy of 10 kV. A low-energy electron flood gun is utilized for charge neutralization in the analysis mode.

The sputter source used is a 5-keV SF5+ cluster source also operated at an incident angle of 45° to the surface normal. For thin model samples on Si, the SF5+ current is maintained at ˜2.7 nA with a 750 um×750 um raster. For the thick samples on coupons and for the samples on stents, the current is maintained at 6 nA with a 500 um×500 um raster. All primary beam currents are measured with a Faraday cup both prior to and after depth profiling.

All depth profiles are acquired in the noninterlaced mode with a 5-ms pause between sputtering and analysis. Each spectrum is averaged over a 7.37 second time period. The analysis is immediately followed by 15 seconds of SF5+ sputtering. For depth profiles of the surface and subsurface regions only, the sputtering time was decreased to 1 second for the 5% active agent sample and 2 seconds for both the 25% and 50% active agent samples.

Temperature-controlled depth profiles are obtained using a variable-temperature stage with Eurotherm Controls temperature controller and IPSG V3.08 software. Samples are first placed into the analysis chamber at room temperature. The samples are brought to the desired temperature under ultra high-vacuum conditions and are allowed to stabilize for 1 minute prior to analysis. All depth profiling experiments are performed at −100 C and 25 C.

### Atomic Force Microscopy (AFM)

AFM is a high resolution surface characterization technique. AFM is used in the art to provide topographical imaging, in addition when employed in Tapping Mode™ can image material and or chemical properties for example imaging drug in an aggregated state. Additionally cross-sectioned samples can be analyzed.

A stent as described herein is obtained. AFM may be employed as described in Ranade et al., “Physical characterization of controlled release of paclitaxel from the TAXUS Express2 drug-eluting stent” J. Biomed. Mater. Res. 71 (4):625-634 (2004) incorporated herein in its entirety by reference.

Polymer and drug morphologies, coating composition, at least may be determined using atomic force microscopy (AFM) analysis. A multi-mode AFM (Digital Instruments/Veeco Metrology, Santa Barbara, Calif.) controlled with Nanoscope Ma and NanoScope Extender electronics is used. TappingMode™ AFM imaging may be used to show topography (a real-space projection of the coating surface microstructure) and phase-angle changes of the AFM over the sample area to contrast differences in the materials properties.

### Example 15

### Determination of the Blood Concentration of an Active Agent

This assay can be used to demonstrate the relative efficacy of a therapeutic compound delivered from a device of the invention to not enter the blood stream and may be used in conjunction with a drug penetration assay (such as is described in PCT/US2006/010700, incorporated in its entirety herein by reference). At predetermined time points (e.g. 1 d, 7 d, 14 d, 21 d, and 28 d, or e.g. 6 hrs, 12 hrs, 24 hrs, 36 hrs, 2 d, 3 d, 5 d, 7 d, 8 d, 14 d, 28 d, 30 d, and 60 d), blood samples from the subjects that have devices that have been implanted are collected by any art-accepted method, including venipuncture. Blood concentrations of the loaded therapeutic compounds are determined using any art-accepted method of detection, including immunoassay, chromatography (including liquid/liquid extraction HPLC tandem mass spectrometric method (LC-MS/MS), and activity assays. See, for example, Ji, et al., “96-Well liquid-liquid extraction liquid chromatography-tandem mass spectrometry method for the quantitative determination of ABT-578 in human blood samples” Journal of Chromatography B. 805:67-75 (2004) incorporated in its entirety herein by reference.

In one test, blood samples are collected by venipuncture into evacuated collection tubes containing editic acid (EDTA) (n=4). Blood concentrations of the active agent (e.g. rapamycin) are determined using a validated liquid/liquid extraction HPLC tandem pass mass spectormetric method (LC-MS/MS) (Ji et al., et al., 2004). The data are averaged, and plotted with time on the x-axis and blood concetration of the drug is represented on the y-axis in ng/ml.

### Example 16

### Preparation of Supercritical Solution Comprising Poly(Lactic-Co-Glycolic Acid) (PLGA) in Hexafluoropropane

A view cell at room temperature (with no applied heat) is pressurized with filtered 1,1,1,2,3,3-Hexafluoropropane until it is full and the pressure reaches 4500 psi. Poly(lactic-co-glycolic acid) (PLGA) is added to the cell for a final concentration of 2 mg/ml. The polymer is stirred to dissolve for one hour. The polymer is fully dissolved when the solution is clear and there are no solids on the walls or windows of the cell.

### Example 17

### Dry Powder Rapamycin Coating on an Electrically Charged L605 Cobalt Chromium Metal Coupon

A 1 cm×2 cm L605 cobalt chromium metal coupon serving as a target substrate for rapamycin coating is placed in a vessel and attached to a high voltage electrode. Alternatively, the substrate may be a stent or another biomedical device as described herein, for example. The vessel (V), of approximately 1500 cm³ volume, is equipped with two separate nozzles through which rapamycin or polymers could be selectively introduced into the vessel. Both nozzles are grounded. Additionally, the vessel (V) is equipped with a separate port was available for purging the vessel. Upstream of one nozzle (D) is a small pressure vessel (PV) approximately 5 cm³ in volume with three ports to be used as inlets and outlets. Each port is equipped with a valve which could be actuated opened or closed. One port, port (1) used as an inlet, is an addition port for the dry powdered rapamycin. Port (2), also an inlet is used to feed pressurized gas, liquid, or supercritical fluid into PV. Port (3), used as an outlet, is used to connect the pressure vessel (PV) with nozzle (D) contained in the primary vessel (V) with the target coupon.

Dry powdered Rapamycin obtained from LC Laboratories in a predominantly crystalline solid state, 50 mg milled to an average particle size of approximately 3 microns, is loaded into (PV) through port (1) then port (1) is actuated to the closed position. The metal coupon is then charged to +7.5 kV using a Glassman Series EL high-voltage power source. The drug nozzle on port has a voltage setting of −7.5 kV. After approximately 60-seconds, the drug is injected and the voltage is eliminated. Upon visual inspection of the coupon using an optical microscope, the entire surface area of the coupon is examined for relatively even distribution of powdered material. X-ray diffraction (XRD) is performed as described herein to confirm that the powdered material is largely crystalline in nature as deposited on the metal coupon. UV-Vis and FTIR spectroscopy is performed as describe herein to confirm that the material deposited on the coupon is rapamycin.

### Example 18

### Polymer Coating on an Electrically Charged L605 Coupon Using Rapid Expansion from a Liquefied Gas

A coating apparatus as described in example 17 above is used in the foregoing example. In this example the second nozzle, nozzle (P), is used to feed precipitated polymer particles into vessel (V) to coat a L605 coupon. Alternatively, the substrate may be a stent or another biomedical device as described herein, for example. Nozzle (P) is equipped with a heater and controller to minimize heat loss due to the expansion of liquefied gases. Upstream of nozzle (P) is a pressure vessel, (PV2), with approximately 25-cm³ internal volume. The pressure vessel (PV2) is equipped with multiple ports to be used for inlets, outlets, thermocouples, and pressure transducers. Additionally, (PV2) is equipped with a heater and a temperature controller. Each port is connected to the appropriate valves, metering valves, pressure regulators, or plugs to ensure adequate control of material into and out of the pressure vessel (PV2). One outlet from (PV2) is connected to a metering valve through pressure rated tubing which was then connected to nozzle (P) located in vessel (V). In the experiment, 150 mg of poly(lactic-co-glycolic acid) (PLGA) is added to pressure vessel (PV2). 1,1,1,2,3,3-hexafluoropropane is added to the pressure vessel (PV2) through a valve and inlet. Pressure vessel (PV2) is set at room temperature with no applied heat and the pressure is 4500 psi. Nozzle (P) is heated to 150° C. A 1-cm×2-cm L605 coupon is placed into vessel (V), attached to an electrical lead and heated via a heat block 110° C. Nozzle (P) is attached to ground. The voltage is set on the polymer spray nozzle and an emitter=pair beaker to a achieve a current greater than or equal to 0.02 mAmps using a Glassman high-voltage power source at which point the metering valve is opened between (PV2) and nozzle (P) in pressure vessel (PV). Polymer dissolved in liquefied gas and is fed at a constant pressure of 200 psig into vessel (V) maintained at atmospheric pressure through nozzle (P) at an approximate rate of 3.0 cm³/min. After approximately 5 seconds, the metering valve is closed discontinuing the polymer-solvent feed. Vessel (V) is Nitrogen gas for 30 seconds to displace the fluorocarbon. After approximately 30 seconds, the metering valve is again opened for a period of approximately 5 seconds and then closed. This cycle is repeated about 4 times. After an additional 1-minute the applied voltage to the coupon was discontinued and the coupon was removed from pressure vessel (V). Upon inspection by optical microscope, a polymer coating is examined for even distribution on all non-masked surfaces of the coupon.

### Example 19

### Dual Coating of a Metal Coupon with Crystalline Rapamycin and Poly(Lactic-Co-Glycolic Acid) (PLGA)

An apparatus described in example 17 and further described in example 18 is used in the foregoing example. In preparation for the coating experiment, 25 mg of crystalline powdered rapamycin with an average particle size of 3-microns is added to (PV) through port (1), then port (1) was closed. Next, 150 mg of poly(lactic-co-glycolic acid) (PLGA) is added to pressure vessel (PV2). 1,1,1,2,3,3-hexafluoropropane is added to the pressure vessel (PV2) through a valve and inlet. Pressure vessel (PV2) is kept at room temperature with no applied heat with the pressure inside the isolated vessel (PV2) approximately 4500 psi. Nozzle (P) is heated to 150° CA 1-cm×2-cm L605 coupon is added to vessel (V) and connected to a high-voltage power lead. Both nozzles (D) and (P) are grounded. To begin, the coupon is charged to +7.5 kV after which port (3) connecting (PV) containing rapamycin to nozzle (D) charged at −7.5 kV is opened allowing ejection of rapamycin into vessel (V) maintained at ambient pressure. Alternatively, the substrate may be a stent or another biomedical device as described herein, for example. After closing port (3) and approximately 60-seconds, the metering valve connecting (PV2) with nozzle (P) inside vessel (V) is opened allowing for expansion of liquefied gas to a gas phase and introduction of precipitated polymer particles into vessel (V) while maintaining vessel (V) at ambient pressure. After approximately 15 seconds at a feed rate of approximately 3 cm³/min., the metering valve s closed while the coupon remained charged. The sequential addition of drug followed by polymer as described above is optionally repeated to increase the number of agent and polymer layers after which the applied potential is removed from the coupon and the coupon was removed from the vessel. The coupon is then examined using an optical microscopeto to determine whether a consistent coating is visible on all surfaces of the coupon except where the coupon was masked by the electrical lead.

### Example 20

### Dual Coating of a Metal Coupon with Crystalline Rapamycin and Poly(Lactic-Co-Glycolic Acid) (PLGA) Followed by Supercritical Hexafluoropropane Sintering

After inspection of the coupon created in example 19, the coated coupon (or other coated substrate, e.g. coated stent) is carefully placed in a sintering vessel that is at a temperature of 75° C. 1,1,1,2,3,3-hexafluoropropane in a separate vessel at 75 psi is slowly added to the sintering chamber to achieve a pressure of 23 to 27 psi. This hexafluoropropane sintering process is done to enhance the physical properties of the film on the coupon. The coupon remains in the vessel under these conditions for approximately 10 min after which the supercritical hexafluoropropane is slowly vented from the pressure vessel and then the coupon was removed and reexamined under an optical microscope. The coating is observed in conformal, consistent, and semi-transparent properties as opposed to the coating observed and reported in example 19 without dense hexafluoropropane treatment. The coated coupon is then submitted for x-ray diffraction (XRD) analysis, for example, as described herein to confirm the presence of crystalline rapamycin in the polymer.

### Example 21

### Coating of a Metal Cardiovascular Stent with Crystalline Rapamycin and Poly(Lactic-Co-Glycolic Acid) (PLGA)

The apparatus described in examples 17, 18 and 20 is used in the foregoing example. The metal stent used is made from cobalt chromium alloy of a nominal size of 18 mm in length with struts of 63 microns in thickness measuring from an abluminal surface to a luminal surface, or measuring from a side wall to a side wall. The stent is coated in an alternating fashion whereby the first coating layer of drug is followed by a layer of polymer. These two steps, called a drug/polymer cycle, are repeated twice so there are six layers in an orientation of agent and polymer-agent and polymer-drug-polmer. After completion of each polymer coating step and prior the application of the next drug coating step, the stent is first removed from the vessel (V) and placed in a small pressure vessel where it is exposed to supercritical hexafluoropropane as described above in example 20.

### Example 22

### Layered Coating of a Cardiovascular Stent with an Anti-Restenosis Therapeutic and Polymer in Layers to Control Drug Elution Characteristics

A cardiovascular stent is coated using the methods described in examples 10 and 11 above. The stent is coated in such as way that the drug and polymer are in alternating layers. The first application to the bare stent is a thin layer of a non-resorbing polymer, approximately 2-microns thick. The second layer is a therapeutic agent with anti-restenosis indication. Approximately 35 micrograms are added in this second layer. A third layer of polymer is added at approximately 2-microns thick, followed by a fourth drug layer which is composed of about 25 micrograms of the anti-restenosis agent. A fifth polymer layer, approximately 1-micron thick is added to stent, followed by the sixth layer that includes the therapeutic agent of approximately 15-micrograms. Finally, a last polymer layer is added to a thickness of about 2-microns. After the coating procedure, the stent is annealed using carbon dioxide as described in example 16 above. In this example a drug eluting stent (DES) is described with low initial drug “burst” properties by virtue of a “sequestered drug layering” process, not possible in conventional solvent-based coating processes. Additionally, by virtue of a higher concentration of drug at the stent ‘inter-layer’ the elution profile is expected to reach as sustained therapeutic release over a longer period of time.

### Example 23

### Layered Coating of a Cardiovascular Stent with an Anti-Restenosis Therapeutic and an Anti-Thrombotic Therapeutic in a Polymer

A cardiovascular stent is coated as described in example 11 above. In this example, after a first polymer layer of approximately 2-microns thick, a drug with anti-thrombotic indication is added in a layer of less than 2-microns in thickness. A third layer consisting of the non-resorbing polymer is added to a thickness of about 4-microns. Next another drug layer is added, a different therapeutic, with an anti-restenosis indication. This layer contains approximately 100 micrograms of the anti-restenosis agent. Finally, a polymer layer approximately 2-microns in thickness is added to the stent. After coating the stent is treated as described in example 20 to sinter the coating using hexafluoropropane.

### Example 24

### Coating of Stent with Rapamycin and Poly(Lactic-Co-Glycolic Acid) (PLGA)

Micronized Rapamycin is purchased from LC Laboratories. 50:50 PLGA (Mw=˜90) are purchased from Aldrich Chemicals. Eurocor CoCr (7 cell) stents are used. The stents are coated by dry electrostatic capture followed by supercritical fluid sintering, using 3 stents/coating run and 3 runs/data set. Analysis of the coated stents is performed by multiple techniques on both stents and coupons with relevant control experiments described herein.

In this example, PLGA is dissolved in 1,1,1,2,3,3-Hexafluoropropane with the following conditions: a) room temperature, with no applied heat; b) 4500 psi; and c) at 2 mg/ml concentration. The spray line is set at 4500 psi, 150° C. and nozzle temperature at 150° C. The solvent (Hexafluoropropane) is rapidly vaporized when coming out of the nozzle (at 150° C.). A negative voltage is set on the polymer spray nozzle to achieve a current of greater than or equal to 0.02 mAmps. The stent is loaded and polymer is sprayed for 15 seconds to create a first polymer coating.

The stent is then transferred to a sintering chamber that is at 75° C. The solvent, in this example 1, 1,2,3,3-hexafluoropropane, slowly enters the sintering chamber to create a pressure at 23 to 27 psi. Stents are sintered at this pressure for 10 minutes.

11.5 mg Rapamycin is loaded into the Drug injection port. The injection pressure is set at 280 psi with +7.5 kV for the stent holder and −7.5 kV for the drug injection nozzle. After the voltage is set for 60 s, the drug is injected into the chamber to create a first drug coating.

A second polymer coating is applied with two 15 second sprays of dissolved polymer with the above first polymer coating conditions. The second coating is also subsequently sintered in the same manner.

A second drug coating is applied with the same parameters as the first drug coating. Lastly, the outer polymer layer is applied with three 15 second sprays of dissolved polymer with the above polymer coating conditions and subsequently sintered.

### Example 25

### Stent Strut Fracture and Coating Penetration Simulated Testing and Durability Testing

Stent stut breakage and coating resistance of the coating to penetration by the strut may be demonstrated in-vitro using fatigue cyclic loading of the coated stent which mimics the stresses and strains that occur in use of the stent (due to internal and/or external forces such as blood flow and pressure and/or normal daily movements of a person), and may also and/or alternatively include a simulation of the delivery and expansion of the stent for placement in a lumen. For example, the testing may be conducted in accordance with ASTM F2477-07 “Standard Test Methods forin vitro Pulsatile Durability Testing of Vascular Stents.” In some embodiments, the fatigue testing may be “challenge tested” which may mean testing conducted at over expansion and/or to longer cycles than the intended life cycle of the stent in order to induce a fracture of a strut to show whether or not the coating was penetrated by the fractured strut. In any case, visusal inspection as noted elsewhere herein is used (for example using SEM, and/or Optical Microscopy) or as indicated in ASTM F2477, in order to inspect the stent for fractures, and then in order to evaluate the coating for penetration (complete or as a percentage of the coating thickness at the particular fracture location). This may include inspecting the coated stent prior to expansion, then at multiple time points thereafter in order to evaluate any fracture and coating penetration.

In some embodiments, the where a stut fracture has occurred during testing according to ASTM F2477-07 (i.e. to typical duration of 10 years of equivalent use (at 72 beats per minute), or at least 380 million cycles), but the coating has not been penetrated completely thereby, the coating is substantially resistant to stent strut breakage. Thus, there is no requirement for additional challenge testing. If however, there is no stent strut breakage in this period and at these conditions, then an alternative test may be to submit the stent to further testing to induce a stent strut breakage and to evaluate the coating thereafter as noted herein.

Additionally, the coatings as described herein may substantially prevent stent strut breakage, i.e. provide durability to the stent. For example, where a stut fracture has not occurred during testing according to ASTM F2477-07 (i.e. to typical duration of 10 years of equivalent use (at 72 beats per minute), or at least 380 million cycles), equivalently produced uncoated stents (same lot and sized stents) may be tested at the same conditions to determine if there is any stent breakage of the uncoated stents. If there is stent breakage of the equivalently produced (same lot and sized stents) stents, then the coating may be deemed to substantially prevent stent strut breakage. Sufficient stents (coated and/or uncoated) should be tested to ensure that there is at least an improvement of 10% in stent breakage (coated stent better than uncoated stent) with 90% confidence and 90% reliability. Sufficient stents (coated and/or uncoated) should be tested to ensure that there is at least an improvement of 25% in stent breakage (coated stent better than uncoated stent) with 90% confidence and 90% reliability. Sufficient stents (coated and/or uncoated) should be tested to ensure that there is at least an improvement of 30% in stent breakage (coated stent better than uncoated stent) with 90% confidence and 90% reliability. Sufficient stents (coated and/or uncoated) should be tested to ensure that there is at least an improvement of 40% in stent breakage (coated stent better than uncoated stent) with 90% confidence and 90% reliability. Sufficient stents (coated and/or uncoated) should be tested to ensure that there is at least an improvement of 50% in stent breakage (coated stent better than uncoated stent) with 90% confidence and 90% reliability. Sufficient stents (coated and/or uncoated) should be tested to ensure that there is at least an improvement of 60% in stent breakage (coated stent better than uncoated stent) with 90% confidence and 90% reliability. Sufficient stents (coated and/or uncoated) should be tested to ensure that there is at least an improvement of 75% in stent breakage (coated stent better than uncoated stent) with 90% confidence and 90% reliability.

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for preparing a coated stent comprising the following steps:
providing a stent;
forming a coating comprising a pharmaceutical agent and a polymer on the stent wherein at least part of the pharmaceutical agent is in crystalline form, wherein forming the coating comprises depositing a fiber reinforcement on the stent, wherein the length of the fiber is up to 5 micrometers, and wherein the coating is substantially resistant to stent strut breakage.

2. The method of claim 1, wherein the fiber reinforcement is a natural fiber.

3. The method of claim 1, wherein the fiber reinforcement is a synthetic fiber.

4. The method of claim 1, wherein the fiber reinforcement is deposited on the stent by a Rapid Expansion of Supercritical Solutions (RESS) process.

5. The method of claim 1, wherein the fiber reinforcement is deposited on the stent in dry form.

6. The method of claim 1, wherein the length of the fiber is 200 nanometers to 5 micrometers.

7. The method of claim 1, wherein the fiber comprises a length to diameter ratio of 3:1.

8. The method of claim 1, wherein forming the coating comprises depositing at least one of the pharmaceutical agent and the polymer in dry powder form.

9. The method of claim 8, wherein forming the coating comprises depositing a plurality of layers on said stent to form said coated stent.

10. The method of claim 9, wherein the plurality of layers comprises at least one polymer layer and at least one pharmaceutical agent layer.

11. The method of claim 10, wherein the fiber reinforcement is part of the polymer layer.

12. The method of claim 10, wherein the fiber reinforcement is part of the pharmaceutical agent layer.

13. The method of claim 10, wherein forming the coating comprises depositing alternate pharmaceutical agent and polymer layers.

14. The method of claim 1, wherein the polymer comprises at least one bioabsorbable polymer.

15. The method of claim 14, wherein the bioabsorbable polymer is selected from PLGA (poly(lactide-co-glycolide); DLPLA—poly(dl-lactide); LPLA—poly(1-lactide); PGA—polyglycolide; PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); PDO-PGA-TMC—poly(glycolide-co-trimethylene carbonate-co-dioxanone) and combinations thereof.

16. The method of claim 1, wherein the pharmaceutical agent comprises a macrolide immunosuppressive (limus) drug.

17. The method of claim 16, wherein the macrolide immunosuppressive drug comprises one or more of rapamycin, 4O—O-(2-Hydroxyethyl)rapamycin (everolimus), 4O—O-Benzyl-rapamycin, 4O—O-(4′-Hydroxymethyl)benzyl-rapamycin, 4O—O-[4′-(1,2-Dihydroxyethyl)]benzyl-rapamycin, 4O—O-Allyl-rapamycin, 4O—O-[3′-(2,2-Dimethyl-1,3-dioxolan-4(S)-yl)-prop-2′-en-1′-yl]-rapamycin, (2′:E,4′S)-4O—O-(4′,5′-Dihydroxypent-2′-en-1′-yl)-rapamycin, 4O—O-(2-Hydroxy)ethoxycar-bonylmethyl-rapamycin, 4O—O-(3-Hydroxy)propyl-rapamycin, 4O—O-(6-Hydroxy)hexyl-rapamycin, 4O-[2-(2-Hydroxy)ethoxy]ethyl-rapamycin, 4O—O-[(3S)-2,2-Dimethyldioxolan-3-yl]methyl-rapamycin, 4O—O-[(2S)-2,3-Dihydroxyprop-1-yl]-rapamycin, 4O—O-(2-Acetoxy)ethyl-rapamycin, 4O—O-(2-Nicotinoyloxy)ethyl-rapamycin, 4O—O-[2-(N-Morpholino)acetoxy]ethyl-rapamycin, 4O—O-(2-N-Imidazolylacetoxy)ethyl-rapamycin, 4O—O-[2-(N-Methyl-N′-piperazinyl)acetoxy]ethyl-rapamycin, 39-O-Desmethyl-39,40-O,O-ethylene-rapamycin, (26R)-26-Dihydro-4O—O-(2-hydroxy)ethyl-rapamycin, 28-O-Methyl-rapamycin, 4O—O-(2-Aminoethyl)-rapamycin, 4O—O-(2-Acetaminoethyl)-rapamycin, 4O—O-(2-Nicotinamidoethyl)-rapamycin, 4O—O-(2-(N-Methyl-imidazo-2′-ylcarbethoxamido)ethyl)-rapamycin, 4O-(2-Ethoxycarbonylaminoethylkapamycin, 4O—O-(2-Tolylsulfonamidoethyl)-rapamycin, 4O—O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), and 4O-epi-(N-1-tetrazolyl)-rapamycin (zotarolimus).

18. The method of claim 1, comprising forming a silane layer on the stent, and covalently attaching heparin to the silane layer.

19. The method of claim 18, wherein onset of heparin anti-coagulant activity is obtained at week 3 or later, and wherein heparin anti-coagulant activity remains at an effective level for at least one of: 90 days after onset of heparin activity, at least 120 days after onset of heparin activity, at least 200 days after onset of heparin activity.

20. The method of claim 8, wherein the forming coating is done when the stent is in a collapsed state.

21. The method of claim 8, wherein said coated stent has a radial expansion ratio of about 1 in a collapsed state and at least one of: up to about 3.0 in the expanded state, up to about 3.0 in the expanded state, up to about 4.0 in the expanded state, up to about 5.0 in the expanded state, up to about 6.0 in the expanded state, over about 3.0 in the expanded state, and over about 4.0 in the expanded state.
